(19) United States
(12) Patent Application Publication
(10) Pub. No.: US 2023/0341371 A1
(43) Pub. Date: Oct. 26, 2023

(54) BLOOD ANALYSIS METHOD

(71) Applicant: SEKISUI MEDICAL CO., LTD., Chuo-ku (JP)

(72) Inventors: Toshiki KAWABE, Chuo-ku (JP); Yukio ODA, Chuo-ku (JP)

(73) Assignee: SEKISUI MEDICAL CO., LTD., Chuo-ku (JP)

(21) Appl. No.: 17/998,876

(22) PCT Filed: Jul. 15, 2021
(86) PCT No.: PCT/IB2021/056379

§ 371 Date: Nov. 15, 2022

(30) Foreign Application Priority Data
May 15, 2020 (JP) 2020-086344

(51) Int. Cl. G01N 33/49

(52) U.S. Cl. CPC G01N 33/4905

(57) ABSTRACT

Provided is a blood analysis method including: acquiring coagulation reaction data on a blood specimen; calculating a parameter related to a centroid point from a differential curve of the coagulation reaction data; and evaluating coagulation properties of the blood specimen using the parameter related to the centroid point.

### FIELD OF THE INVENTION

The present invention relates to a blood analysis method.

### BACKGROUND OF THE INVENTION

A blood coagulation test is a test for diagnosing blood coagulation properties of a patient by measuring a blood coagulation time and the like with a prescribed reagent added to a blood specimen of the patient. A representative example of the blood coagulation test includes measurement of prothrombin time (PT), activated partial thromboplastin time (APTT), or thrombin time. An abnormality in the blood coagulation test (elongation of the blood coagulation time) is caused by influence of an anticoagulant, decrease of a component involved in coagulation, congenital deficiency of a blood coagulation factor, presence of an autoantibody inhibiting the coagulation reaction, and the like.

In the blood coagulation test, a coagulation reaction curve can be obtained by measuring, over time, a blood coagulation reaction amount caused after adding a reagent to a blood specimen. The coagulation reaction curve is varied in shape in accordance with the type of an abnormality of the blood coagulation system (Non Patent Literature 1). There is a known method for evaluating coagulability of a blood specimen based on a coagulation reaction curve. For example, Patent Literature 1, Patent Literature 2, and Patent literature 3 disclose that parameters such as a maximum coagulation rate, a maximum coagulation acceleration rate, and a maximum coagulation deceleration rate are obtained from a coagulation reaction curve to evaluate coagulability of a blood specimen based on these parameters. Patent Literature 4 describes a method for determining severity of hemophilia based on an average rate of change in a coagulation rate until a time when a coagulation reaction of the patient reaches a maximum coagulation rate or a maximum coagulation acceleration rate. Patent Literature 5 describes a method for determining presence of a coagulation factor VIII (FVIII) inhibitor based on a ratio between patient’s plasma and control plasma in the slope of a line indicating a coagulation time against a plasma dilution factor. Patent Literature 6 describes a method for evaluating coagulation function of a blood specimen including calculating a centroid point of a coagulation reaction rate curve, and evaluating a concentration of a coagulation-involved component or coagulation abnormality using information based on the centroid point. The centroid point and a centroid velocity actually used in Patent Literature 6 are, however, what is called a weighted average point and a weighted average velocity. Patent Literature 7 describes a method for evaluating coagulation function of a blood specimen including calculating a peak width at a prescribed height in a coagulation reaction rate curve, and determining a concentration of a coagulation-involved component or coagulation abnormality using information based on the peak width.

When elongation of APTT is found in a blood specimen, a cross-mixing test is generally performed to determine a factor of the elongation of APTT. For example, it is determined which of a coagulation factor inhibitor (anticoagulation factor), a lupus anticoagulant (LA), and coagulation factor deficiency such as hemophilia causes the elongation of APTT. In the cross-mixing test, each of normal plasma, test plasma, and mixed plasmas respectively containing the test plasma and the normal plasma in various volume ratios is measured for APTT immediately after mixing (immediate reaction) and for APTT after incubation at 37° C. for 2 hours (delayed reaction) (see Patent Literature 2). Measured values are plotted in a graph having the APTT measured values (in seconds) as the ordinate, and the volume ratio between the test plasma and the normal plasma as the abscissa. Each of the thus created graphs of the immediate reaction and the delayed reaction shows, in accordance with the factor of the elongation of APTT, a pattern of “downward convex,” “straight line,” or “upward convex.” Based on these patterns of the immediate reaction and the delayed reaction, the factor of the elongation of APTT is determined. For example, when a reaction curve of “downward convex” is obtained in the immediate reaction, the factor of coagulation delay is an inhibitor or factor deficiency, but it cannot be determined which is the factor. In this case, when the curve of the delayed reaction is “downward convex”, it can be determined that the factor of the coagulation delay is the factor deficiency, and when it is “straight line” or “upward convex,” it can be determined that the factor of the coagulation delay is the inhibitor. When a reaction curve of “upward convex” is obtained in the immediate reaction, the factor of the coagulation delay is the inhibitor or LA, but it cannot be determined which is the factor. In this case, when the pattern of the delayed reaction is more definite “upward convex” than in the immediate reaction, it can be determined that the factor of the coagulation delay is the inhibitor.

When it is determined, by the cross-mixing test, that the APTT elongation is caused by a coagulation factor inhibitor, an inhibitor titer is measured by the Bethesda method in general. In the Bethesda method, after a sample obtained by mixing a dilution series of test plasma, and normal plasma is heated at 37° C. for 2 hours, residual activity of the coagulation factor in the sample is measured, and the titer of the inhibitor of the coagulation factor is calculated based on a calibration curve of measured values. The Bethesda method is a standard quantitative method for the titer of inhibitors against coagulation factor VIII (FVIII) and factor IX (FIX) .

### CITATION LIST

### Patent Literature

[Patent Literature 1] JP-A-2016-194426
[Patent Literature 2] JP-A-2016-118442
[Patent Literature 3] JP-A-2017-106925
[Patent Literature 4] JP-A-2018-017619
[Patent Literature 5] JP-A-2018-517150
[Patent Literature 6] JP-A-2019-086518
[Patent Literature 7] JP-A-2019-086517

### Non Patent Literature

[Non Patent Literature 1] British Journal of Haematology, 1997, 98: 68-73

### SUMMARY OF THE INVENTION

It is desirable that blood coagulation properties can be evaluated more simply or in a shorter time by using measurement data of (test) items analyzed in a laboratory. The present invention provides a method for obtaining information on blood coagulation properties such as a coagulation time elongation factor, a coagulation factor concentration, and a titer of a coagulation factor inhibitor based on a coagulation reaction curve.

The present inventors have found that information on blood coagulation properties can be obtained simply and in a short time by calculating a parameter related to a centroid point based on a first or secondary differential curve of a coagulation reaction curve.

The present invention provides the following:
A blood analysis method, comprising:
   (1) acquiring coagulation reaction data on a subject blood specimen;
   (2) calculating a parameter related to a centroid point from a differential curve of the coagulation reaction data; and
   (3) evaluating coagulation properties of the blood specimen using the parameter related to the centroid point.
The method according to [1], wherein the centroid point is at least one selected from the group consisting of a centroid point in a prescribed region of a primary differential curve of a coagulation reaction curve of the blood specimen, and a centroid point in a prescribed region of a secondary differential curve of the coagulation reaction curve.
The method according to [2],
wherein the centroid point in the prescribed region of the primary differential curve is represented by coordinates (vTg, vHg) defined by a centroid time vTg and a centroid height vHg, and
the parameter related to the centroid point includes one or more parameters of the centroid point related to the centroid point in the prescribed region of the primary differential curve selected from the group consisting of the centroid height vHg, a centroid peak width vWg, a B flattening vABg, a W flattening vAWg, and a W time rate vTWg, wherein,
assuming that the primary differential curve is F(t) (wherein t is time), that times when F(t) has a prescribed value X are t1 and t2 (wherein t1 < t2), and that when n = t2 - t1 + 1, and b = X, vTg and vHg are represented by the following expressions:
$$\text{v} T \text{g} = \frac{{\sum }_{i = t 1}^{t 2} \left(i \times F \left(i\right)\right)}{{\sum }_{i = t 1}^{t 2} F \left(i\right)} \qquad \text{(1)}$$
$$\text{v} H \text{g} = \frac{\left({\sum }_{i = t 1}^{t 2} F \left(i\right) ∗ F \left(i\right)\right) - \left(n ∗ b ∗ b\right)}{2 ∗ \left\{\left({\sum }_{i = t 1}^{t 2} F \left(i\right)\right) - n ∗ b\right\}} \qquad \text{(2)}$$
wherein vWg represents a time length satisfying F(t) ≥ vHg in time from t1 to t2,
vABg represents a ratio between vHg and vB, wherein vB represents a time length satisfying F(t) ≥ X in time from t1 to t2,
vAWg represents a ratio between vHg and vWg, and
vTWg represents a ratio between vTg and vWg.
The method according to [3], wherein the prescribed value X is a value corresponding to from 0.5% to 99% of a maximum value of the primary differential curve F(t).
The method according to any one of [2] to [4], wherein the centroid point in the prescribed region of the secondary differential curve includes one or more selected from the group consisting of a centroid point in a prescribed region of a positive peak of the secondary differential curve, and a centroid point in a prescribed region of a negative peak of the secondary differential curve.
The method according to [5],
wherein the centroid point in the prescribed region of the positive peak of the secondary differential curve is represented by coordinates (pTg, pHg) defined by a centroid time pTg and a centroid height pHg, and
the parameter related to the centroid point includes one or more parameters related to the centroid point in the prescribed region of the positive peak of the secondary differential curve selected from the group consisting of the centroid height pHg, a centroid peak width pWg, a B flattening pABg, a W flattening pAWg, and a W time rate pTWg, wherein,
assuming that the secondary differential curve is F′ (t) (wherein t is time), that times when F′ (t) has a prescribed value X′ are t1 and t2 (wherein t1 < t2), and that when n = t2 - t1 + 1, and b′ = X′, pTg and pHg are represented by the following expressions:
$$\text{p} T \text{g} = \frac{{\sum }_{i = t 1}^{t 2} \left(i \times {F}^{′} \left(i\right)\right)}{{\sum }_{i = t 1}^{t 2} {F}^{′} \left(i\right)} \qquad \text{(1)}$$
$$\text{p} H \text{g} = \frac{\left({\sum }_{i = t 1}^{t 2} {F}^{′} \left(i\right) ∗ {F}^{′} \left(i\right)\right) - \left(n ∗ {b}^{′} ∗ {b}^{′}\right)}{2 ∗ \left\{\left({\sum }_{i = t 1}^{t 2} {F}^{′} \left(i\right)\right) - n ∗ {b}^{′}\right\}} \qquad \text{(2)}$$
wherein pWg represents a time length satisfying F′ (t) ≥ pHg in time from t1 to t2,
pABg represents a ratio between pHg and pB, wherein pB represents a time length satisfying F′ (t) ≥ X′ in time from t1 to t2,
pAWg represents a ratio between pHg and pWg, and
pTWg represents a ratio between pTg and pWg.
The method according to [5],
wherein the centroid point in the prescribed region of the negative peak of the secondary differential curve is represented by coordinates (mTg, mHg) defined by a centroid time mTg and a centroid height mHg, and
the parameter related to the centroid point includes one or more parameters related to the centroid point in the prescribed region of the negative peak of the secondary differential curve selected from the group consisting of the centroid height mHg, a centroid peak width mWg, a B flattening mABg, a W flattening mAWg, and a W time rate mTWg, wherein,
assuming that the secondary differential curve is F′ (t) (wherein t is time), that times when F′ (t) has a prescribed value X″ are t1 and t2 (wherein t1 < t2), and that when n = t2 - t1 + 1, and b″ = X″, mTg and mHg are represented by the following expressions:
$$\text{m} T \text{g} = \frac{{\sum }_{i = t 1}^{t 2} \left(i \times {F}^{′} \left(i\right)\right)}{{\sum }_{i = t 1}^{t 2} {F}^{′} \left(i\right)} \qquad \text{(1)}$$
$$\text{m} H \text{g} = \frac{\left({\sum }_{i = t 1}^{t 2} {F}^{′} \left(i\right) ∗ {F}^{′} \left(i\right)\right) - \left(n ∗ {b}^{″} ∗ {b}^{″}\right)}{2 ∗ \left\{\left({\sum }_{i = t 1}^{t 2} {F}^{′} \left(i\right)\right) - n ∗ {b}^{″}\right\}} \qquad \text{(2)}$$
wherein mWg represents a time length satisfying F′ (t) ≤ mHg in time from t1 to t2,
mABg represents a ratio between mHg and mB, wherein mB represents a time length satisfying F′ (t) ≤ X″ in time from t1 to t2,
mAWg represents a ratio between mHg and mWg, and
mTWg represents a ratio between mTg and mWg.
The method according to [6], wherein the prescribed value X′ is a value corresponding to from 0.5% to 99% of a maximum value of the secondary differential curve F′ (t) .
The method according to [7], wherein the prescribed value X″ is a value corresponding to from 0.5% to 99% of a minimum value of the secondary differential curve F′ (t) .
The method according to any one of [1] to [9], wherein the evaluation of the coagulation properties is measurement of a concentration of a coagulation factor.
The method according to [10], wherein the coagulation factor is at least one selected from the group consisting of coagulation factor VIII and coagulation factor IX.
The method according to any one of [1] to [9], wherein the evaluation of the coagulation properties is evaluation of presence or degree of coagulation abnormality.
The method according to [12], wherein the coagulation abnormality is hemophilia A or hemophilia B.
The method according to any one of [1] to [9], wherein the evaluation of the coagulation properties is evaluation of a coagulation time elongation factor.
The method according to [14], wherein the evaluation of the elongation factor is evaluation of which of coagulation factor deficiency, a lupus anticoagulant, and a coagulation factor inhibitor is the elongation factor.
The method according to any one of [1] to [9], wherein the evaluation of the coagulation properties is measurement of a titer of a coagulation factor inhibitor.
The method according to [16], wherein the coagulation factor inhibitor is a coagulation factor VIII inhibitor.
The method according to any one of [14] to [17], wherein the (1) includes:
   preparing a mixed specimen by mixing a subject blood specimen and a normal blood specimen;
   heating the mixed specimen, and acquiring coagulation reaction data of the heated mixed specimen; and
   acquiring coagulation reaction data of the mixed specimen unheated, the (2) includes:
      calculating, as a first parameter, a parameter related to the centroid point of the mixed specimen unheated; and
   calculating, as a second parameter, a parameter related to the centroid point of the heated mixed specimen, and the (3) includes:
      evaluating coagulation properties of the subject blood specimen based on a ratio or a difference between the first parameter and the second parameter.
The method according to [18], wherein the heating is performed at 30° C. or more and 40° C. or less for 2 to 30 minutes.
The method according to any one of [10] to [13], wherein the (2) includes:
   acquiring a parameter set including a parameter group consisting of parameters related to a centroid point each of which are calculated from different regions of the differential curve, the (3) includes:
      comparing the parameter set of the subject blood specimen with a corresponding parameter set of a template blood specimen, and
      evaluating, based on a result of the comparing, presence or degree of coagulation abnormality in the subject blood specimen, and
      the template blood specimen is a blood specimen in which presence or degree of the coagulation abnormality is known.
The method according to [20], wherein the number of the different regions is from 5 to 50.
A program for performing the blood analysis method according to any one of [1] to [21].
An apparatus for performing the blood analysis method according to any one of [1] to [21].

### Advantageous Effects of Invention

The present invention provides a method enabling evaluation of an elongation factor of a blood specimen or measurement of a coagulation factor concentration simply and in a short time. The method of the present invention enables measurement of a titer of a coagulation factor inhibitor in a shorter time and with higher sensitivity as compared with the conventional Bethesda method. In addition, the method of the present invention is applicable to an automatic analyzer used in a conventional blood coagulation test, and hence can largely reduce effort required for the measurement.

### BRIEF DESCRIPTION OF THE DRAWINGS

FIG. 1 illustrates one embodiment of procedures in a blood analysis method of the present invention.

FIG. 2 illustrates one embodiment of procedures in a data analysis step of FIG. 1.

FIG. 3 illustrates an example of a coagulation reaction curve.

FIG. 4 illustrates an example of a coagulation reaction curve obtained after base line adjustment.

FIG. 5A is a partially enlarged view of an example of the coagulation reaction curve, and FIG. 5B is a partially enlarged view of an example of the coagulation reaction curve obtained after base line adjustment.

FIG. 6 illustrates an example of a coagulation reaction curve having been subjected to correction processing.

FIG. 7A illustrates an example of a corrected linear curve, and FIG. 7B illustrates an example of a corrected quadratic curve.

FIG. 8 is a conceptual diagram explaining a calculation target threshold.

FIG. 9A and FIG. 9B respectively illustrate a centroid point (black square) and a weighted average point (black circle), and a centroid peak width vWg and a weighted average peak width vW (B) of a linear curve obtained when the calculation target threshold is 10% (left) and 50% (right).

FIG. 10A and FIG. 10B respectively illustrate centroid points (black squares) and weighted average points (black circles), and centroid peak widths pWg and mWg and weighted average peak widths pW and mW of a positive peak and a negative peak of a quadratic curve obtained when the calculation target threshold is 50%.

FIG. 11 is a conceptual diagram illustrating an embodiment of a structure of an automatic analyzer used for performing the blood analysis method of the present invention.

FIG. 12 illustrates relationships between a coagulation factor concentration and Vmax, vHg, and vH. Vmax, vHg, and vH are plotted against logarithms of FVIII concentration (left) and FIX concentration (right).

FIG. 13 illustrates a difference between a centroid point and a weighted average point. Centroid points (black squares) and weighted average points (black circles) from different calculation target thresholds are illustrated together with corrected linear curves.

FIG. 13A: normal specimen, FIG. 13B: FVIII deficient plasma, FIG. 13C: FIX deficient plasma.

FIG. 14 illustrates a difference between a centroid point and a weighted average point. vHg (white circles) and vH (black circles) are plotted against a calculation target threshold. FIG. 14A: normal specimen, FIG. 14B: FVIII deficient plasma, FIG. 14C: FIX deficient plasma.

FIGS. 15-1 illustrates distributions of Pa, Pb, Pb/Pa, and Pb - Pa of a parameter vHg60. FVIII: FVIII group, LA: LA group, Inhi.: inhibitor group. A numerical value shown below each diagram is a P value (two-sided T test).

FIGS. 15-2 illustrates distributions of Pa, Pb, Pb/Pa, and Pb - Pa of a parameter pHg60. FVIII: FVIII group, LA: LA group, Inhi.: inhibitor group. A numerical value shown below each diagram is a P value (two-sided T test).

FIGS. 15-3 illustrates distributions of Pa, Pb, Pb/Pa, and Pb - Pa of a parameter mHg60. FVIII: FVIII group, LA: LA group, Inhi.: inhibitor group. A numerical value shown below each diagram is a P value (two-sided T test).

FIGS. 15-4 illustrates distributions of Pa, Pb, Pb/Pa, and Pb - Pa of a parameter vABg5. FVIII: FVIII group, LA: LA group, Inhi.: inhibitor group. A numerical value shown below each diagram is a P value (two-sided T test).

FIGS. 15-5 illustrates distributions of Pa, Pb, Pb/Pa, and Pb - Pa of a parameter APTT. FVIII: FVIII group, LA: LA group, Inhi.: inhibitor group. A numerical value shown below each diagram is a P value (two-sided T test).

FIG. 16A is a plot of Pb/Pa of vHG 30% of a mixed specimen containing a test specimen against a measured value of an inhibitor titer of the test specimen. FIG. 16B illustrates a calibration curve.

FIG. 16C is a plot of calculated values based on the calibration curve of FIG. 16B against the measured value of the inhibitor titer of the test specimen.

FIGS. 16D and 16E are replots in a low titer region.

FIG. 17A is a plot of Pb/Pa of RvABg 20% of a mixed specimen containing a test specimen against a measured value of an inhibitor titer of the test specimen. FIG. 17B illustrates a calibration curve.

FIG. 17C is a plot of calculated values based on the calibration curve of FIG. 17B against the measured value of the inhibitor titer of the test specimen.

FIGS. 17D and 17E are replots in a low titer region.

FIG. 18A is a plot of Pb/Pa of RvAWg 5% of a mixed specimen containing a test specimen against a measured value of an inhibitor titer of the test specimen. FIG. 18B illustrates a calibration curve.

FIG. 18C is a plot of calculated values based on the calibration curve of FIG. 18B against the measured value of the inhibitor titer of the test specimen.

FIGS. 18D and 18E are replots in a low titer region.

FIG. 19A is a plot of Pb/Pa of vTWg 40% of a mixed specimen containing a test specimen against a measured value of an inhibitor titer of the test specimen. FIG. 19B illustrates a calibration curve.

FIG. 19C is a plot of calculated values based on the calibration curve of FIG. 19B against the measured value of the inhibitor titer of the test specimen.

FIGS. 19D and 19E are replots in a low titer region.

FIG. 20A is a plot of Pb/Pa of pAWg 70% of a mixed specimen containing a test specimen against a measured value of an inhibitor titer of the test specimen. FIG. 20B illustrates a calibration curve.

FIG. 20C is a plot of calculated values based on the calibration curve of FIG. 20B against the measured value of the inhibitor titer of the test specimen.

FIGS. 20D and 20E are replots in a low titer region.

FIG. 21A is a plot of Pb/Pa of RmHg 0.5% of a mixed specimen containing a test specimen against a measured value of an inhibitor titer of the test specimen. FIG. 21B illustrates a calibration curve.

FIG. 21C is a plot of calculated values based on the calibration curve of FIG. 21B against the measured value of the inhibitor titer of the test specimen.

FIGS. 21D and 21E are replots in a low titer region.

### DETAILED DESCRIPTION OF THE INVENTION

Evaluation parameters used in conventional blood coagulation property analysis, for example, parameters such as a maximum coagulation rate, a maximum coagulation acceleration rate, and a maximum coagulation deceleration rate calculated based on a coagulation reaction curve, are not sufficient for accurately obtaining information on blood coagulation properties such as a coagulation factor concentration and a coagulation factor inhibitor titer. In a cross-mixing test generally employed in conventional determination of a coagulation time elongation factor, an APTT elongation factor is determined based on a qualitative graph pattern, and hence the determination may be difficult in some graph patterns. In addition, in the cross-mixing test, APTT measurement is required to be performed after subjecting mixed plasma to a heat treatment (incubation) at 37° C. for 2 hours, and hence, this test requires a time as long as about two and a half hours including a heating time and a measurement time.

Besides, the Bethesda method, that is, a standard method employed in conventional measurement of an inhibitor titer, requires much time and effort. In the Bethesda method, measurement requires a time as long as 2 or more hours including time for heating a sample, and in addition, this method is unsuitable for automatic measurement with an analyzer. Furthermore, the above-described time for the cross-mixing test is also required. The Bethesda method is described in “Inhibitor (Anticoagulation Factor) Measurement” in glossary of the Japanese Society on Thrombosis and Hemostasis (www.jsth.org/glossary/) as “A criterion is “not detected,” and a value of 0.5 BU/ml or more is determined as positive.” and “It is difficult to make accurate measurement in a range of 0 to 0.5 BU/ml.,” and hence a lower limit of detection is regarded as 0.5 BU/mL.

### 1. Blood Analysis Method

The present invention relates to a blood analysis method to which waveform analysis is applied. According to the method of the present invention, various properties of blood coagulation of a blood specimen, such as determination of coagulation factor deficiency, presence of an antiphospholipid antibody such as a lupus anticoagulant (LA), presence of a coagulation factor inhibitor, and a blood coagulation time elongation factor, or measurement of concentrations of respective components such as various coagulation factors and coagulation factor inhibitors can be evaluated. Besides, according to the present invention, a test time of a conventional blood coagulation test can be simplified or shortened. For example, in evaluation of presence of a coagulation factor inhibitor in a cross-mixing test, a heating time can be reduced to be shorter than 2 hours, for example, to about 10 minutes. In evaluation of presence of a coagulation factor inhibitor, quantitative determination can be made by obtaining a ratio or a difference between parameters. Furthermore, according to the present invention, a titer of a coagulation factor inhibitor can be measured in an extremely shorter time and with higher sensitivity than in a conventional method.

### 1.1. Outline of Analysis Method

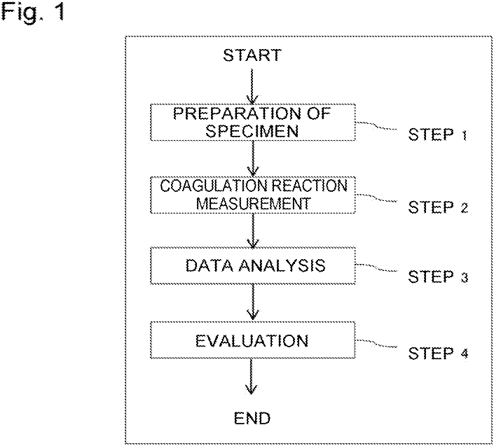

The outline of the blood analysis method of the present invention (hereinafter, also referred to as the method of the present invention) will be described referring to a flow chart of FIG. 1. In the present method, a subject blood specimen (hereinafter, also referred to simply as a specimen) is first prepared (step 1). Next, coagulation reaction measurement of the specimen is executed (step 2). Measurement data thus obtained is analyzed to calculate various parameters related to a coagulation reaction curve (step 3). Based on the parameters thus obtained, coagulation properties and the like of the test specimen are evaluated (step 4).

### 1.2. Coagulation Reaction Measurement

As the test specimen, plasma of a subject is preferably used. To the specimen, an anticoagulant usually used in a coagulation test can be added. For example, after blood is collected with a blood collecting tube containing sodium citrate, the resultant is centrifuged to obtain plasma. The test specimen used in the method of the present invention may be a normal specimen, may be an abnormal specimen having coagulation abnormality, or may be a mixed specimen of these depending on the purpose of the analysis. For example, in measurement of a coagulation factor concentration, a normal specimen or an abnormal specimen is preferably used, and in determination of a coagulation time elongation factor or measurement of an inhibitor titer, a mixed specimen is preferably used.

To the test specimen, a coagulation time measuring reagent is added to start a blood coagulation reaction. A coagulation reaction of a mixed solution caused after adding the reagent is measured (step 2). The reagent to be used can be arbitrarily selected in accordance with a method for the coagulation reaction measurement. An example of the method of the coagulation reaction measurement includes a coagulation reaction measurement method for measuring prothrombin time (PT), activated partial thromboplastin time (APTT), dilute prothrombin time, dilute partial thromboplastin time, kaolin clotting time, dilute Russell’s viper venom time, or a fibrinogen concentration (Fbg), and a coagulation time measuring reagent appropriate for each of the time is used. A coagulation time measuring reagent is commercially available (such as APTT Reagent Coagpia APTT-N; manufactured by Sekisui Medical Co., Ltd.). Hereinafter, the method of the present invention will be described by exemplifying coagulation reaction measurement mainly for measuring activated partial thromboplastin time (APTT). Those skilled in the art can modify the method of the present invention to a coagulation time measurement method for another time (such as measurement of prothrombin time (PT)).

For the measurement of the coagulation reaction, general means, such as optical means for measuring an amount of scattered light, transmittance, absorbance or the like, or mechanical means for measuring a viscosity of plasma, may be employed. A reaction start point of the coagulation reaction can be typically defined as a time point when the coagulation reaction is started by mixing a trigger reagent with a specimen, but another timing may be defined as the reaction start time point. A time period for continuing the measurement of the coagulation reaction can be, for example, about several tens seconds to 7 minutes from the time point of mixing the specimen and the trigger reagent. This measurement time may be set to an arbitrarily determined fixed value, or may be time until detection of the end of the coagulation reaction of the specimen. During the measurement time, measurement of progress of the coagulation reaction (for example, photometry in employing optical detection) can be repeatedly performed at prescribed intervals. The measurement may be performed, for example, at intervals of 0.1 seconds. The temperature of the mixed solution during the measurement is under normal conditions, for example, 30° C. or more and 40° C. or less, and preferably 35° C. or more and 39° C. or less. Various conditions for the measurement can be appropriately set in accordance with the test specimen, the reagent, the measurement means, and the like.

A series of operations in the coagulation reaction measurement can be performed with an automatic analyzer. An example of the automatic analyzer includes Blood Coagulation Automatic Analyzer CP3000 (manufactured by Sekisui Medical Co., Ltd.). Alternatively, some of the operations may be manually performed. For example, with a test specimen manually prepared, the subsequent operations can be performed with the automatic analyzer.

### 1.3. Data Analysis

### 1.3.1. Base Line Adjustment and Correction Processing of Data

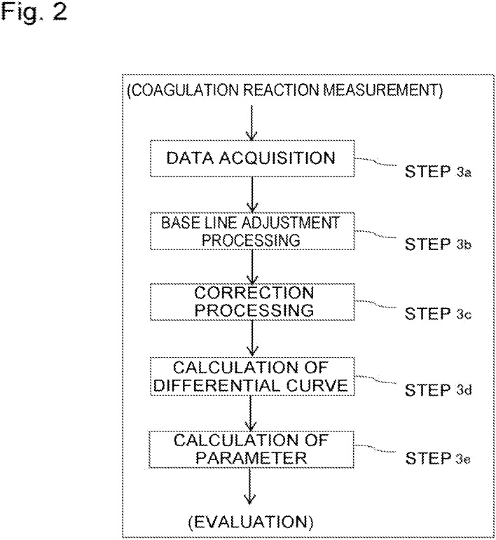

Next, data obtained in the coagulation reaction measurement is analyzed (step 3). The data analysis performed in step 3 will now be described. One embodiment of a flow of the data analysis is illustrated in FIG. 2. The data analysis of step 3 may be performed in parallel with the coagulation reaction measurement of step 2, or may be performed afterward by using data of the precedently performed coagulation reaction measurement.

In step 3a, measurement data of the coagulation reaction measurement is acquired. This data is, for example, data reflecting coagulation reaction process of the specimen obtained in the APTT measurement of step 2 described above. For example, data corresponding to change over time of the amount of progress of the coagulation reaction (for example, the amount of scattered light) in the mixed solution of the specimen and the coagulation time measuring reagent after adding a calcium chloride solution (trigger reagent) is acquired. Such data obtained through the coagulation reaction measurement is herein designated also as coagulation reaction data.

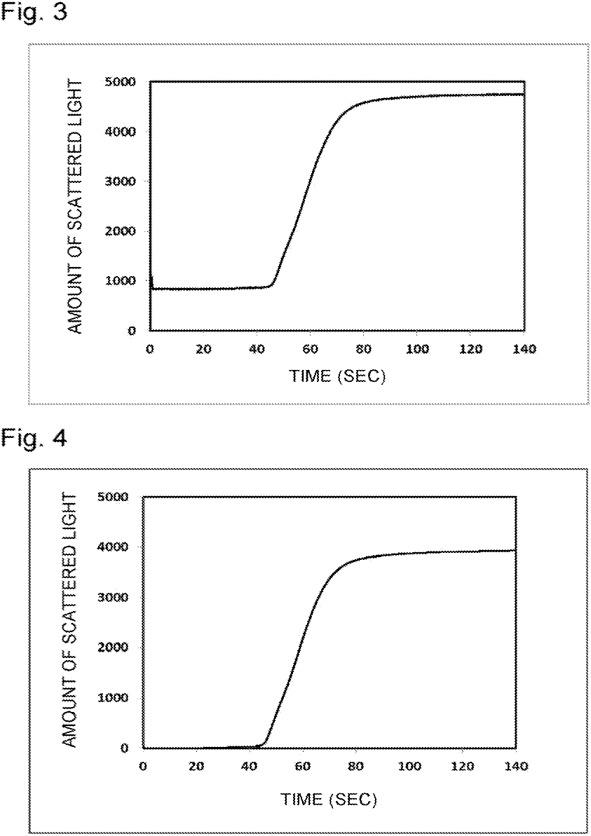

An example of the coagulation reaction data acquired in step 3a is illustrated in FIG. 3. FIG. 3 illustrates a coagulation reaction curve based on the amount of scattered light, in which the abscissa indicates elapsed time after the addition of the calcium chloride solution (coagulation reaction time), and the ordinate indicates the amount of scattered light. The coagulation reaction of the specimen proceeds as the time elapses, and hence the amount of scattered light increases. Herein, such a curve indicating change of the coagulation reaction amount against the coagulation reaction time, indicated by the amount of scattered light or the like, is designated as a coagulation reaction curve.

A coagulation reaction curve based on the amount of scattered light as illustrated in FIG. 3 is usually in a sigmoidal shape. On the other hand, a coagulation reaction curve based on the amount of transmitted light is usually in a reverse sigmoidal shape. Hereinafter, description will be given on data analysis using, as the coagulation reaction data, a coagulation reaction curve based on the amount of scattered light. It will be obvious to those skilled in the art that similar processing can be performed also in data analysis using, as the coagulation reaction data, a coagulation reaction curve based on the amount of transmitted light or absorbance. Alternatively, as the coagulation reaction data, a coagulation reaction curve obtained by mechanical means such as change of a viscosity of the mixed solution may be analyzed.

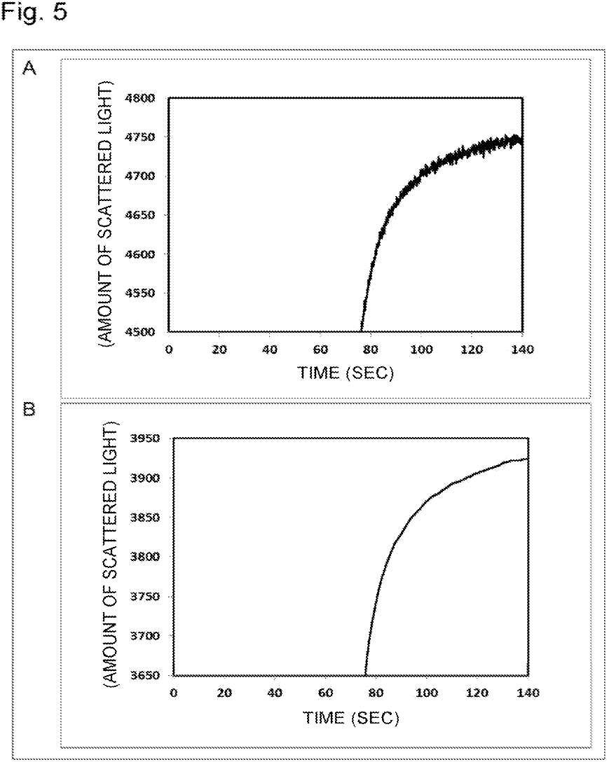

In step 3b, base line adjustment of the coagulation reaction curve is performed. The base line adjustment includes smoothing processing for removing a noise, and zero adjustment. FIG. 4 illustrates an example of the coagulation reaction curve of FIG. 3 having been subjected to the base line adjustment (smoothing processing and zero adjustment). For the smoothing processing, any one of known noise removal methods can be employed. As illustrated in FIG. 3, the mixed solution containing the specimen essentially scatters light, and hence the amount of scattered light is larger than 0 at the measurement start point (at time 0). Through the zero adjustment following the smoothing processing, the amount of scattered light at time 0 is adjusted to 0 as illustrated in FIG. 4. FIGS. 5A and 5B are partially enlarged views of the coagulation reaction curve of FIG. 3 obtained respectively before and after the base line adjustment. In FIG. 5B, the smoothing processing and the zero adjustment have been conducted on the data of FIG. 5A.

The height of the coagulation reaction curve depends on a fibrinogen concentration in the specimen. On the other hand, there are individual differences in the fibrinogen concentration, and hence the height of the coagulation reaction curve is varied depending on the specimen. Accordingly, in the present method, correction processing for relativizing the coagulation reaction curve after the base line adjustment is performed in step 3c if necessary. Through the correction processing, a coagulation reaction curve independent of the fibrinogen concentration can be obtained, and thus, a difference in shape of the coagulation reaction curve after the base line adjustment can be quantitatively compared between specimens.

In one embodiment, the coagulation reaction curve after the base line adjustment is corrected, in the correction processing, to have a maximum value of a prescribed value. Preferably, in the correction processing, a corrected coagulation reaction curve P(t) is obtained from the coagulation reaction curve after the base line adjustment in accordance with the following expression. In the expression, D(t) represents the coagulation reaction curve after the base line adjustment, Dmax and Dmin respectively represent a maximum value and a minimum value of D(t), Drange represents a change width of D(t) (namely, Dmax - Dmin), and A represents a maximum value of the corrected coagulation reaction curve.

$$\text{P} \left(\text{t}\right) = \left[\left(\text{D} \left(\text{t}\right) - \text{Dmin}\right) / \text{Drange}\right] \times \text{A}$$

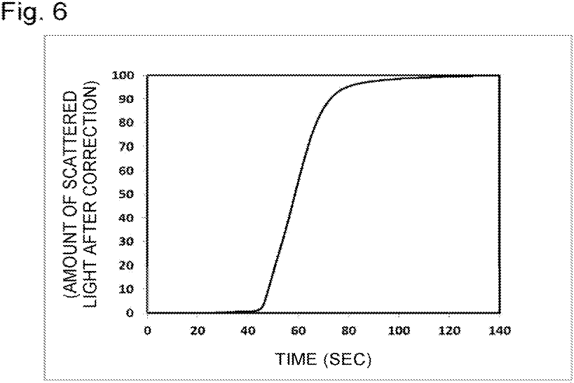

As an example, FIG. 6 illustrates data obtained by correcting the coagulation reaction curve of FIG. 4 to have a maximum value of 100. Although the correction is performed in FIG. 6 so as to make corrected values range from 0 to 100, other values (for example, from 0 to 10,000, namely, A = 10,000 in the expression (1)) may be employed. This correction processing is not always necessary.

Alternatively, the correction processing as described above may be performed on a differential curve described below, or a parameter calculated based on the differential curve. In other words, a differential curve of the coagulation reaction curve D(t) obtained after the base line adjustment without the correction processing is calculated, and then, the resultant can be converted to a value corresponding to P(t). Alternatively, a parameter is calculated from the differential curve, and then the parameter value can be converted to a value corresponding to P(t).

### 1.3.2. Calculation of Differential Curve

In step 3d, a differential curve resulting from differentiation of the coagulation reaction curve is calculated. Herein, examples of the differential curve include a primary differential curve obtained by one differentiation of the coagulated reaction curve (obtained with or without the correction processing), and a secondary differential curve obtained by two differentiations (or one differentiation of the primary differential curve) of the coagulation reaction curve. The primary differential curve encompasses an uncorrected primary differential curve (coagulation rate curve), and a corrected primary differential curve. The coagulation rate curve indicates values obtained by one differentiation of the coagulation reaction curve (obtained without the correction processing), namely, a change rate of the coagulation reaction amount during an arbitrary coagulation reaction time (coagulation rate). The corrected primary differential curve indicates values obtained by one differentiation of the coagulated reaction curve (obtained with the correction processing), namely, a relative change rate of the coagulation reaction amount during an arbitrary coagulation reaction time (herein, sometimes referred to as a coagulation progress rate). Accordingly, the primary differential curve can be a waveform corresponding to the coagulation rate or a relative value thereof in the coagulation reaction of the specimen.

The secondary differential curve is obtained by two differentiations of a coagulation reaction curve (obtained with or without the correction processing). A secondary differential curve derived from the coagulation reaction curve (obtained without the correction processing) is also designated as a coagulation acceleration rate curve, and indicates a coagulation acceleration rate against the coagulation reaction time. A secondary differential curve derived from the coagulation reaction curve (obtained with the correction processing) is also designated as a corrected secondary differential curve, and indicates a rate of change over time of the coagulation progress rate.

Herein, a coagulation reaction curve obtained with the correction processing, and a coagulation reaction curve obtained without the correction processing are respectively designated as a corrected zero order curve and an uncorrected zero order curve, which are also generically designated as a “zero order curve.” Herein, the corrected zero order curve, and a primary differential curve of the uncorrected zero order curve are respectively designated as a corrected linear curve and an uncorrected linear curve, which are also generically designated as a “linear curve.” Besides, herein, the corrected zero order curve and a secondary differential curve of the uncorrected zero order curve, or the corrected linear curve and a primary differential curve of the uncorrected linear curve are respectively designated as a corrected quadratic curve, or an uncorrected quadratic curve, which are also generically designated as a “quadratic curve.”

Herein, a value corresponding to progress of coagulation obtained based on a linear curve is, no matter whether or not the correction processing is performed on an originating coagulation reaction curve, also generically designated as a first derivative value. Herein, a value corresponding to a change rate of a first derivative value obtained based on a quadratic curve is, no matter whether or not the correction processing is performed on an originating coagulation reaction curve, also generically designated as a second derivative value.

The differentiation of a zero order curve and a linear curve can be performed by a known method. FIG. 7A illustrates a corrected linear curve obtained by subjecting the corrected zero order curve of FIG. 6 to one differentiation. The abscissa of FIG. 7A indicates the coagulation reaction time, and the ordinate indicates the first derivative value. FIG. 7B illustrates a corrected quadratic curve obtained by subjecting the corrected linear curve of FIG. 7A to one differentiation. The abscissa of FIG. 7B indicates the coagulation reaction time, and the ordinate indicates the second derivative value.

### 1.3.3. Calculation of Parameter

In step 3e, a parameter characterizing the linear curve or the quadratic curve is calculated. In calculation process of a parameter from the linear curve or the quadratic curve, one or more prescribed regions are extracted from the curve while for each of one or more prescribed regions, a parameter characterizing the prescribed region is calculated. As a result, for each of the one or more prescribed regions, one or more parameters characterizing the prescribed region can be calculated. More specifically, a parameter obtained from the linear curve or the quadratic curve is a parameter related to the centroid point of the prescribed region of the linear curve or the quadratic curve of a specimen. This parameter will now be described.

### 1.3.3.1. Extraction of Calculation Target Region

In the calculation of the parameter, one or more prescribed regions are first extracted from the linear curve. Hereinafter, the prescribed region used in the parameter calculation is also designated as a calculation target region. The calculation target region is a region (segment) in which a first derivative value (y value) of the linear curve is equal to or larger than a prescribed calculation target threshold X. In other words, the calculation target region is a region (segment) in which the first derivative value (y value) of the linear curve is equal to or larger than the prescribed calculation target threshold X, and is equal to or smaller than the maximum value.

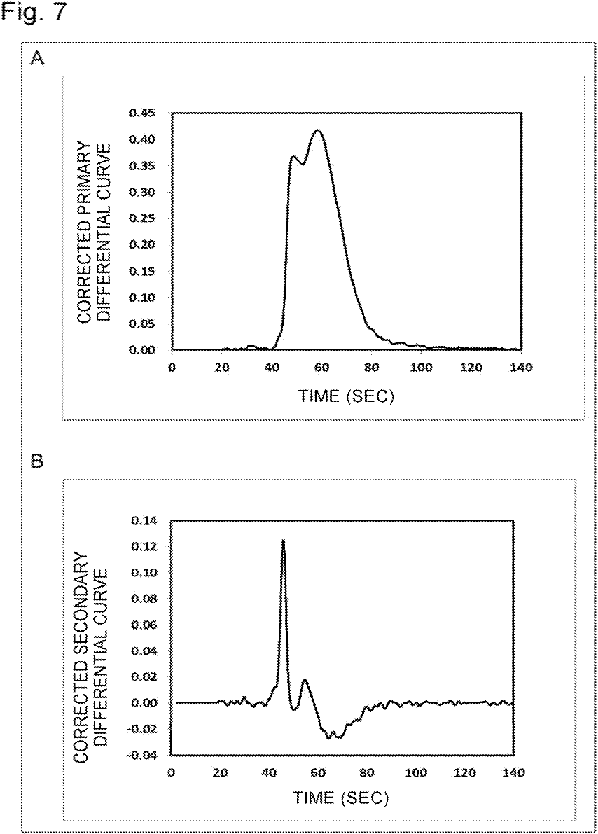

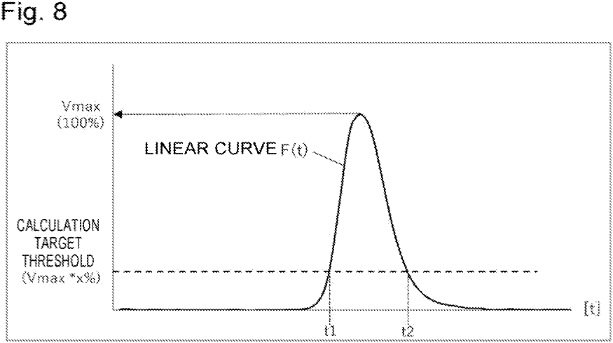

More specifically, assuming that the differential curve (linear curve) is F(t) (wherein t is time), and that the maximum value of F(t) is Vmax, the calculation target region is a region (segment) of the F(t) in which F(t) ≥ Vmax × x% is satisfied. More specifically, the calculation target region is a region (segment) of the linear curve (F(t) in which Vmax ≥ F(t) ≥ Vmax × x%. Accordingly, “Vmax × x%” is the calculation target threshold X, and corresponds to a lower limit value of the calculation target region. Hereinafter, the “Vmax × x%” of the calculation target threshold may be sometimes indicated simply as x%. The calculation target region will be described referring to FIG. 8. FIG. 8 illustrates the linear curve F(t) (wherein t is time), and the maximum value Vmax of the F(t). In addition, a base line corresponding to Vmax × x% is illustrated with a dotted line, and time point t1 and t2 when F(t) = Vmax × x% are illustrated. The calculation target region is a region in which the F(t) is equal to or higher than the base line, and is equal to or lower than Vmax (F(t) ≥ Vmax × x%, and t1 ≤ t ≤ t2).

In the method of the present invention, one or more calculation target regions may be extracted. The number of calculation target regions extracted in the method of the present invention is not necessarily limited. When a plurality of calculation target regions are to be extracted, the plurality of calculation target regions are regions different from one another.

### 1.3.3.2. Centroid Point

The centroid point of the calculation target region will now be described. The centroid point can be represented by coordinates on a two-dimensional surface using time t as the abscissa and coagulation reaction data as the ordinate. The centroid point (vTg, vHg) of the calculation target region can be obtained by the following procedure: First, assuming that the maximum value of the linear curve F(t) is Vmax, and that the calculation target threshold is Vmax × x%, time t [t1, ..., and t2] (t1 < t2) when F(t) ≥ Vmax × x × 0.01 is satisfied is obtained. vTg and vHg are calculated respectively in accordance with the following expressions (1) and (2). In the expressions, n = t2 - t1 + 1, and b = Vmax × x%.

$$\text{v} T \text{g} = \frac{{\sum }_{i = t 1}^{t 2} \left(i \times F \left(i\right)\right)}{{\sum }_{i = t 1}^{t 2} F \left(i\right)} \qquad \text{(1)}$$

$$\text{v} H \text{g} = \frac{\left({\sum }_{i = t 1}^{t 2} F \left(i\right) ∗ F \left(i\right)\right) - \left(n ∗ b ∗ b\right)}{2 ∗ \left\{\left({\sum }_{i = t 1}^{t 2} F \left(i\right)\right) - n ∗ b\right\}} \qquad \text{(2)}$$

vTg represents time (t) corresponding to the centroid point of the linear curve, and is herein also designated as the centroid time. vHg represents a first derivative value corresponding to the centroid point of the linear curve, and is herein also designated as the centroid height.

With respect to a quadratic curve, a centroid point, a centroid time, and a centroid height can be similarly defined. The quadratic curve has, as illustrated in FIG. 7B, peaks on both the positive side and the negative side of the second derivative value. Therefore, the centroid point of the quadratic curve can be calculated with respect to both of the positive peak and the negative peak. For example, as for the positive peak, assuming that the maximum value of the quadratic curve A = F′(t) is Amax, and that the calculation target threshold is Amax × x%, time t [t1, ..., and t2] (t1 < t2) when F′(t) ≥ Amax × x × 0.01 is satisfied is obtained, and in accordance with the following expressions (1)′ and (2)′ (wherein n = t2 - t1 + 1, and b = Amax × x%), the centroid time pTg and a centroid height pHg of the positive peak are calculated. As for the negative peak, assuming that the minimum value of the quadratic curve A = F′ (t) is Amin, and that the calculation target threshold is Amin × x%, time t [t1, ..., and t2] (t1 < t2) when F′ (t) ≥ Amin × x × 0.01 is satisfied is obtained, and in accordance with the following expressions (1)″ and (2)″ (wherein n = t2 - t1 + 1, and b = Amin × x%), the centroid time mTg and the centroid height mHg of the negative peak are calculated. In accordance with change of the calculation target threshold, the position of the centroid point changes.

$$\text{p} T \text{g} = \frac{{\sum }_{i = t 1}^{t 2} \left(i \times {F}^{′} \left(i\right)\right)}{{\sum }_{i = t 1}^{t 2} {F}^{′} \left(i\right)} \qquad \text{(1)}$$

$$\text{p} H \text{g} = \frac{\left({\sum }_{i = t 1}^{t 2} {F}^{′} \left(i\right) ∗ {F}^{′} \left(i\right)\right) - \left(n ∗ b ∗ b\right)}{2 ∗ \left\{\left({\sum }_{i = t 1}^{t 2} {F}^{′} \left(i\right)\right) - n ∗ b\right\}} \qquad \text{(2)}$$

$$\text{m} T \text{g} = \frac{{\sum }_{i = t 1}^{t 2} \left(i \times {F}^{′} \left(i\right)\right)}{{\sum }_{i = t 1}^{t 2} {F}^{′} \left(i\right)} \qquad \text{(1)}$$

$$\text{m} H \text{g} = \frac{\left({\sum }_{i = t 1}^{t 2} {F}^{′} \left(i\right) ∗ {F}^{′} \left(i\right)\right) - \left(n ∗ b ∗ b\right)}{2 ∗ \left\{\left({\sum }_{i = t 1}^{t 2} {F}^{′} \left(i\right)\right) - n ∗ b\right\}} \qquad \text{(2)}$$

### 1.3.3.3. Centroid Peak Width

Based on the centroid point, a centroid peak width of the linear curve or the quadratic curve can be calculated. First, the minimum value (t1) and the maximum value (t2) of the time t [t1, ..., and t2] when F(t) ≥ Vmax × x% is satisfied respectively represent the minimum value and the maximum value of the coagulation reaction time in the calculation target region of the linear curve, and these may be sometimes designated respectively as the region start time vTs and the region end time vTe (vTs < vTe). The centroid peak width vWg represents a peak width of the linear curve in which F(t) ≥ vHg is satisfied (time length satisfying F(t) ≥ vHg in time from vTs to vTe).

Similarly, with respect to the positive peak of the quadratic curve, the minimum value and the maximum value of the time when F′ (t) ≥ Amax × x% is satisfied are respectively pTs and pTe, and time length satisfying F′ (t) ≥ pHg in time from pTs to pTe is defined as a centroid peak width pWg. With respect to the negative peak of the quadratic curve, the minimum value and the maximum value of the time when F′ (t) ≤ Amin × x% is satisfied are respectively mTs and mTe, and time length satisfying F′(t) ≤ mHg in time from mTs to mTe is defined as a centroid peak width mWg.

### 1.3.3.4. Flattening and Time Rate

Based on the above-described centroid point, a flattening and a time rate of the linear curve or the quadratic curve can be calculated. First, time length satisfying F(t) ≥ Vmax × x% in time from vTs to vTe is defined as a peak width vB of the linear curve. Similarly, as for the positive peak of the quadratic curve, time length satisfying F′ (t) ≥ Amax × x% in time from pTs to pTe is defined as a peak width pB, and as for the negative peak of the quadratic curve, time length satisfying F′ (t) ≤ Amin × x% in time from mTs to mTe is defined as a peak width mB.

The flattening of the linear curve can be a flattening vABg based on the peak width vB (B flattening), and a flattening vAWg based on the centroid peak width vWg (W flattening). As shown in the following expressions (3a) and (3b), vABg is defined as a ratio between the centroid height vHg and the peak width vB, and vAWg is defined as a ratio between the centroid height vHg and the centroid peak width vWg.

$$\text{vABg =} \text{vHg} / \text{vB} \qquad \text{(3a)}$$

$$\text{vAWg} \text{=} \text{vHg} / \text{vWg} \qquad \text{(3b)}$$

A time rate of the linear curve can be a time rate vTBg based on the peak width vB (B time rate), and a time rate vTWg based on the centroid peak width vWg (W time rate). As shown in the following expressions (4a) and (4b), vTBg is defined as a ratio between the centroid time vTg and the peak width vB, and vTWg is defined as a ratio between the centroid time vTg and the centroid peak width vWg.

$$\text{vTBg} \text{=} \text{vTg} / \text{vB} \qquad \text{(4a)}$$

$$\text{vTWg} \text{=} \text{vTg} / \text{vWg} \qquad \text{(4b)}$$

As for the flattening, vABg = vB/vHg or vAWg = vWg/vHg may hold. As for the time rate, vTBg = vB/Vtg or vTWg = vWg/vTg may hold. Besides, these ratios may be multiplied by a constant K. In other words, for example, as for the flattening, vABg = (vHg/vB)K, vABg = (vB/vHg)K, vAWg = (vHg/vWg)K, or vAWg = (vWg/vHg)K may hold, and as for the time rate, vTBg = (vTg/vB)K, vTBg = (vB/vTg)K, vTWg = (vTg/vWg)K, or vTWg = (vWg/vTg)K may hold (wherein K is a constant).

Similarly, the flattening and the time rate can be obtained with respect to a quadratic curve. For example, as for the positive peak of the quadratic curve, a B flattening pABg based on the peak width or a W flattening pAWg based on the centroid peak width can be obtained as a ratio between pHg and pB or pWg, and a B time rate pTBg based on the peak width or a W time rate pTWg based on the centroid peak width can be obtained as a ratio between pTg and pB or pWg. Similarly, as for the negative peak of the quadratic curve, a B flattening mABg based on the peak width or a W flattening mAWg based on the centroid peak width can be obtained as a ratio between mHg and mB or mWg, and a B time rate mTBg based on the peak width or a W time rate mTWg based on the centroid peak width can be obtained as a ratio between mTg and mB or mWg.

### 1.3.3.5. Parameters Derived From Different Calculation Target Regions

Herein, in order to distinguish parameters derived from different calculation target regions, the respective parameters may be sometimes designated as vTgx, vHgx, and the like in accordance with the originating calculation target thresholds (% to Vmax). For example, when the calculation target threshold is 50% of Vmax, vTg, vHg, vWg, vB, vTs, vTe, vABg, vAWg, vTBg, and vTWg are respectively vTg50%, vHg50%, vWg50%, vB50%, vTs50%, vTe50%, vABg50%, vAWg50%, vTBg50%, and vTWg50%, and pTg, pHg, pWg, pB, pTs, pTe, pABg, pAWg, pTBg, and pTWg are respectively pTg50%, pHg50%, pWg50%, pB50%, pTs50%, pTe50%, pABg50%, pAWg50%, pTBg50%, and pTWg50%, and mTg, mHg, mWg, mB, mTs, mTe, mABg, mAWg, mTBg, and mTWg are respectively mTg50%, mHg50%, mWg50%, mB50%, mTs50%, mTe50%, mABg50%, mAWg50%, mTBg50%, and mTWg50% (or alternatively, % may be omitted to designate these simply as vTg50, vHg50 and the like). Here, vABgx is calculated from vBx and vHgx, vAWgx is calculated from vWgx and vHgx, vTBgx is calculated from vBx and vTgx, and vTWgx is calculated from vWgx and vTgx. This also applies to pABgx, pAWgx, pTBgx, pTWgx, mABgx, mAWgx, mTBgx, and mTWgx.

The parameters vTgx, vHgx, vWgx, vABgx, vAWgx, vTBgx, and vTWgx related to the centroid point of the linear curve, and the parameters pTgx, pHgx, pWgx, pABgx, pAWgx, pTBgx, pTWgx, mTgx, mHgx, mWgx, mABgx, mAWgx, mTBgx, and mTWgx related to the centroid point of the quadratic curve can be used as the parameters characterizing the linear curve or the quadratic curve in the blood analysis of the present invention.

### 1.3.3.6. Comparison With Parameter Related to Weighted Average Point

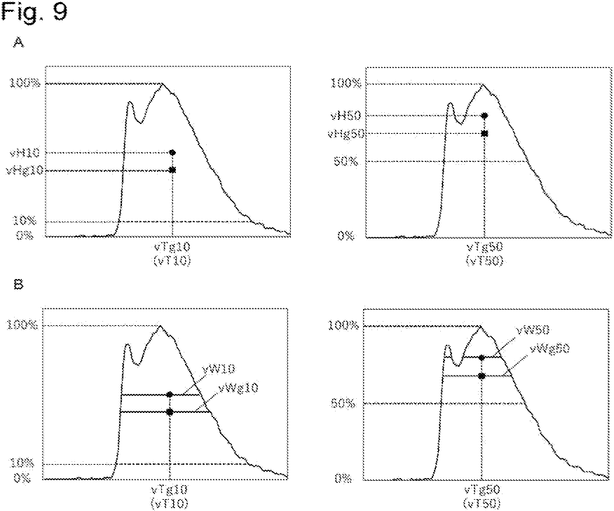

FIG. 9A illustrates a centroid point of a calculation target region of a linear curve obtained when the calculation target threshold is 10% (left) or 50% (right) of Vmax (= 100%). A black square indicates a centroid point. In accordance with the change of the calculation target threshold, the position of the centroid point changes.

FIG. 9A also illustrates a weighted average point (see Patent Literature 6) (black circle) of the calculation target region of the linear curve. The weighted average point (vT, vH) of the linear curve F(t) is represented by the following expressions:

$$\text{v} T = \frac{{\sum }_{i = t 1}^{t 2} \left(i \times F \left(i\right)\right)}{{\sum }_{i = t 1}^{t 2} F \left(i\right)} \qquad \text{(5)}$$

$$\text{v} H = \frac{{\sum }_{i = t 1}^{t 2} \left(i \times F \left(i\right)\right)}{{\sum }_{i = t 1}^{t 2} i} \qquad \text{(6)}$$

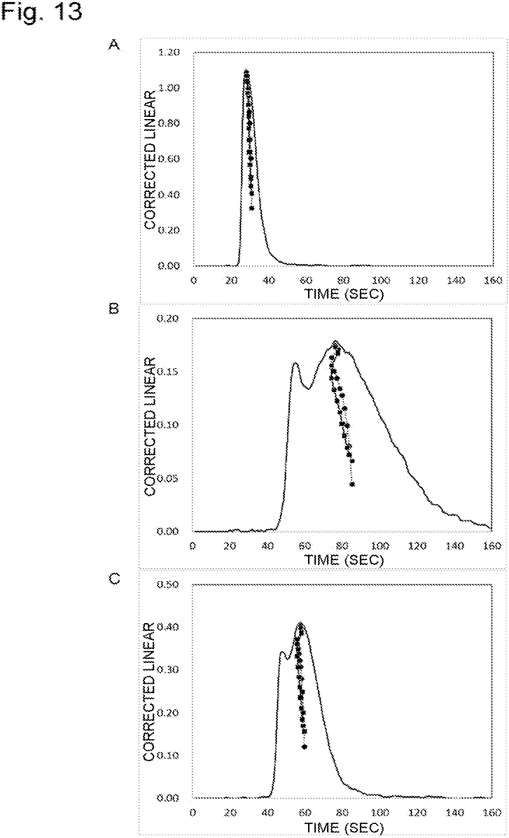

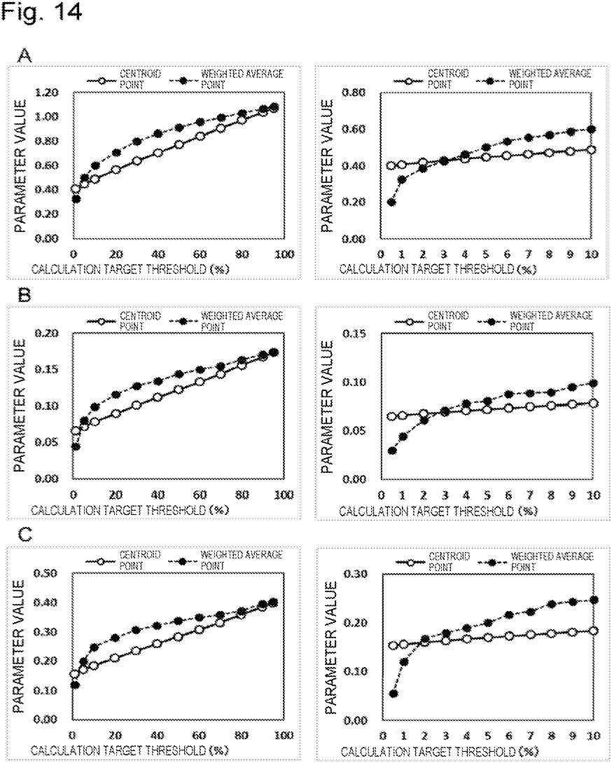

As is obvious from the above-described expressions, FIG. 9A, and FIGS. 13 to 14 described below, although vTgx = vTx, vHgx and vHx can be different from each other, and as a result, the centroid point and the weighted average point can be positioned in different points. Besides, FIG. 9B illustrates a centroid peak width vWg and a weighted average peak width vW in the calculation target region of the linear curve obtained when the calculation target threshold is 10% (left) or 50% (right) of Vmax (= 100%). In the drawing, a black square indicates a centroid point, and a black circle indicates a weighted average point. The weighted average peak width vW represents a peak width of the linear curve satisfying F(t) ≥ vH (time length satisfying F(t) = vH in time from vTs to vTe), and has a different value from vWg. Accordingly, other parameters related to the centroid point based on vHg or vWg (for example, a B flattening vABg, a W flattening vAWg, and a W time rate vTWg of the linear curve) can be also different from parameters related to the weighted average point in the same calculation target region (for example, a B flattening, a W flattening, and a W time rate calculated from the weighted average point of the linear curve).

FIG. 10 illustrates a centroid point, a weighted average point (FIG. 10A), and a centroid peak width vWg and a weighted average peak width vW (FIG. 10B) in calculation target regions of the positive peak and the negative peak of the quadratic curve. The calculation target threshold is 50% of Amax or Amin. Also with respect to the quadratic curve, the centroid point and the weighted average point are positioned in different points, and vWg and vW have different values.

### 1.3.3.7 Other Parameters

The parameters related to the centroid point described above can be used in the blood analysis of the present invention in combination with other parameters characterizing the linear curve or the quadratic curve. Examples of the other parameters include the maximum value Vmax of the linear curve, the maximum value Amax and the minimum value Amin of the quadratic curve, times respectively corresponding thereto (respectively designated as VmaxT, AmaxT, and AminT), the peak widths vB, pB, and mB, the region start times vTs, pTs, and mTs, and the region end times vTe, pTe, and mTe described above.

In the present invention, other examples of the other parameters usable in combination with the parameter related to the centroid point include parameters vT, vH, vAB(vH/vB), vAW(vH/vW), vTB(vT/vB), vTW(vT/vW), pT, pH, pAB(pH/pB), pAW (pH/pW), pTB(pT/pB), pTW(pT/pW), mT, mH, mAB(mH/mB), mAW(mH/mW), mTB(mT/mB), and mTW(mT/mW) related to the weighted average point of the calculation target region of the linear curve or the quadratic curve. The parameters related to the weighted average point can be designated as vTx, vHx and the like in accordance with the calculation target threshold. For example, when the calculation target threshold is 50% of Vmax, vT and vH are designated respectively as vT50% and vH50%. Alternatively, % may be omitted to designate these simply as vT50, vH50, and the like. Hereinafter, % may be omitted in similar description.

In the present invention, still another example of the other parameters usable in combination with the parameter related to the centroid point includes an area under the curve (AUC) in the calculation target region of the linear curve or the quadratic curve. The AUC can encompass an AUC of the linear curve (vAUC), and AUCs of the positive peak and the negative peak of the quadratic curve (respectively pAUC and mAUC). The AUC may be designated as AUCx in accordance with the calculation target threshold. For example, when the calculation target threshold is 50% of Vmax, vAUC, pAUC and mAUC are respectively vAUC50%, pAUC50%, and mAUC50%. In the present invention, still another example of the other parameters usable in combination with the parameter related to the centroid point includes a coagulation time Tc. The coagulation time Tc refers to a reaction elapsed time corresponding to the amount of scattered light of c%, assuming that the amount of scattered light obtained when the change in the amount of scattered light on the zero order curve satisfies a prescribed condition is 100%. c may be an arbitrary value, and for example, c is from 5 to 95.

In the present invention, still other examples of the other parameters usable in combination with the parameter related to the centroid point include an average time vTa, an average height vHa, and a region center time vTm. vTa, vHa, and vTm are represented respectively by the following expressions, wherein the number of data points from F(vTs) to F(vTe) is n. vTa, vHa, and vTm can be designated respectively as vTax, vHax, and vTmx in accordance with the calculation target threshold. For example, when the calculation target threshold is 50% of Vmax, vTa, vHa, and vTm are respectively vTa50%, vHa50%, and vTm50%.

$$v T a = \frac{{\sum }_{i = v T s}^{v T e} i}{n} \qquad \text{(7)}$$

$$v H a = \frac{{\sum }_{i = v T s}^{v T e} F \left(i\right)}{n} \qquad \text{(8)}$$

$$v T m = \frac{v T s + v T e}{2} \qquad \text{(9)}$$

It has been found that the above-described other parameters can be used as parameters for evaluation of a coagulation time elongation factor, evaluation of the presence or degree of coagulation abnormality, or measurement of concentrations of respective components such as various coagulation factors and coagulation factor inhibitors (Patent Literatures 6 and 7, PCT/JP2019/044943, PCT/JP2020/003796, and PCT/JP2020/017507, all of which are incorporated herein as reference).

The series of parameters described above may be parameters derived from corrected coagulation reaction curves (corrected zero order to quadratic curves), or parameters derived from uncorrected coagulation reaction curves (uncorrected zero order to quadratic curves).

The parameter related to the centroid point may be obtained from corrected linear to quadratic curves, or may be obtained from uncorrected linear to quadratic curves. For example, in a corrected linear curve, the coagulation rate is relativized, and some blood coagulation abnormalities can be reflected on the magnitude of the coagulation rate. Accordingly, as some evaluation parameters, preferably parameters related to the coagulation rate, such as the centroid height and the flattening, values obtained from uncorrected linear to quadratic curves may reflect the blood coagulation properties better than values obtained from corrected linear to quadratic curves.

The parameters related to the coagulation reaction curves have been described so far on the basis of the coagulation reaction curves based on the amount of scattered light. On the other hand, it is obvious to those skilled in the art that equivalent parameters can be obtained from coagulation reaction curves based on other coagulation measurement means (such as the amount of transmitted light, and absorbance). For example, in a linear curve F(t) obtained from a coagulation reaction curve in a reverse sigmoidal shape based on the amount of transmitted light, positiveness and negativeness are inverted as compared with those based on the amount of scattered light. In such a case, it is obvious to those skilled in the art that signs are inverted in the F(t) in the calculation of parameters, for example, that the maximum value Vmax is replaced with the minimum value Vmin, that the calculation target region is a region in which F(t) ≤ Vmin × x% is satisfied, that vWg is a time length satisfying F(t) ≤ vHg in time from t1 to t2, and the like.

### 1.4. Evaluation

The parameters related to the centroid point described above, namely, the parameters (vTg, vHg, vWg, vABg, vAWg, vTBg, and vTWg) related to the centroid point of the linear curve, and parameters (pTg, pHg, pWg, pABg, pAWg, pTBg, pTWg, mTg, mHg, mWg, mABg, mAWg, mTBg, and mTWg) related to the centroid points of the quadratic curve, reflect properties related to blood coagulation. A combination of the parameters related to the centroid point can also reflect properties related to blood coagulation. In addition, a combination of the parameter related to the centroid point and the other parameters described above can reflect properties related to blood coagulation. For example, results of various operations such as four arithmetic operations of the parameters may reflect properties related to blood coagulation in some cases. Accordingly, based on the parameters related to the centroid point, coagulation properties of a blood specimen can be variously evaluated as evaluation of a blood coagulation time elongation factor and evaluation of the presence or degree of coagulation abnormalities, including deficiency of a coagulation factor, presence of an antiphospholipid antibody such as a lupus anticoagulant, or measurement of concentrations of respective components such as various coagulation factors and coagulation factor inhibitors (step 4).

In one embodiment, the concentration of a component such as a coagulation factor concentration in a blood specimen is measured in the method of the present invention. In one embodiment, the presence or degree of a coagulation abnormality in a blood specimen is evaluated in the method of the present invention. In one embodiment, a blood coagulation time elongation factor (hereinafter, also referred to simply as the elongation factor) in a blood specimen is evaluated in the method of the present invention. In one embodiment, a titer of a coagulation factor inhibitor (anticoagulation factor, hereinafter, also referred to simply as the inhibitor) in a blood specimen is measured in the method of the present invention.

The coagulation reaction curve corresponding to data from which the centroid point is obtained is acquired in usual measurement, such as APTT measurement, performed in a clinical laboratory. Accordingly, the blood analysis method of the present invention can be easily utilized in a clinical environment only by introducing the data analysis method.

### 2. Evaluation of Coagulation Properties

Now, exemplified embodiments for evaluating coagulation properties of a blood specimen using the parameters related to the centroid point will be described.

### 2.1. Coagulation Factor Concentration

In one embodiment, a concentration of a component such as a coagulation factor in a blood specimen is measured by using the parameter related to the centroid point in the method of the present invention. For example, the parameter related to the centroid point may be in correlation with the concentration of a component such as a coagulation factor in some cases. Accordingly, a calibration curve can be created by obtaining the parameter related to the centroid point from a specimen having a known concentration of a component such as a coagulation factor. This calibration curve can be used for measuring the concentration of the component such as the coagulation factor based on the same parameter calculated from a test specimen. Alternatively, an abnormality (such as deficiency) of the coagulation factor concentration in a test specimen can be detected by comparing the parameter related to the centroid point of the test specimen with that of a normal specimen.

Alternatively, a ratio or a difference between parameters related to the centroid point, or between a parameter related to the centroid point and another parameter (hereinafter, also referred to as a parameter ratio or a parameter difference) may be in correlation with the concentration of a component such as a coagulation factor. Examples of the parameter ratio include the flattening vABg and vAWg. An example of the parameter difference includes a difference between a peak width and a centroid peak width. A calibration curve can be created by obtaining such a parameter ratio or parameter difference. When the calibration curve is used, the concentration of the component such as the coagulation factor can be measured based on the same parameter ratio or parameter difference calculated from a test specimen. Alternatively, when a parameter ratio or parameter difference of a test specimen is compared with that of a normal specimen, an abnormality (such as deficiency) of the coagulation factor concentration in the test specimen can be detected.

Examples of the type of the coagulation factor to be measured include one or more selected from the group consisting of coagulation factor V (FV), coagulation factor VIII (FVIII), coagulation factor IX (FIX), coagulation factor X (FX), coagulation factor XI (FXI), and coagulation factor XII (VXII), among which one or more selected from the group consisting of FVIII and FIX are preferred, and FVIII is more preferred.

Preferable examples of the parameter related to the centroid point to be used for measuring a coagulation factor concentration include vHg, vWg, vABg, vAWg, vTWg, pHg, pABg, and pAWg, among which vHg, vWg, vABg, vAWg, and vTWg are preferred, and vHg is further preferred. The calculation target threshold for obtaining these parameters may be in a range of 0% or more and less than 100%, preferably from 0.5 to 99.5%, more preferably from 5 to 95%, further preferably from 5 to 70%, and still further preferably from 5 to 60%.

A calibration curve to be used for measuring the coagulation factor concentration can be created based on the parameter related to the centroid point, the parameter ratio, or the parameter difference obtained from a specimen having a known concentration of the target coagulation factor. A parameter related to the centroid point, a parameter ratio or a parameter difference is obtained from a test specimen, which can be applied to the calibration curve to measure the concentration of the target coagulation factor.

### 2.2. Evaluation of Coagulation Properties with Template Specimen

In one embodiment, coagulation properties are evaluated by using a parameter set including a parameter group consisting of parameters related to the centroid point each of which are calculated from different calculation target regions in the method of the present invention. For example, there may be a high correlation in a parameter related to a centroid point between specimens having similar coagulation properties in some cases. Accordingly, the coagulation properties (such as a coagulation time elongation factor, the presence or degree of abnormality of blood coagulation, and a coagulation factor concentration) of a test specimen can be evaluated by comparing various parameters related to the centroid point between the test specimen and a template specimen having known coagulation properties.

### 2.2.1. Creation of Parameter Set

In the present embodiment, with respect to a test specimen, a parameter set including a parameter group consisting of parameters related to a centroid point each of which are calculated from different calculation target regions is obtained. In the present embodiment, a parameter group refers to a set of parameters consisting of the same type of parameters calculated from different calculation target regions of a linear curve or a quadratic curve, and a parameter set refers to a set of parameters consisting of one or more parameter groups.

The parameter set may include one or more parameter groups of parameters related to any one type of centroid points. In one example, the parameter set includes two or more parameter groups of parameters related to a centroid point. In another example, the parameter set includes one or more parameter groups of parameters related to a centroid point, and further includes one or more parameter groups of the other parameters (such as vB, pB, mB, vTs, pTs, mTs, vTe, pTe, mTe, vTa, vHa, and vTm), and/or one or more other parameters (such as vAUC, pAUC, mAUC, Tc, Vmax, Amax, Amin, VmaxT, AmaxT, and AminT) .

Preferable examples of the parameter related to a centroid point used in the present embodiment include vHg, vWg, vABg, vAWg, vTBg, vTWg, pHg, pWg, pABg, pAWg, pTBg, pTWg, mHg, mWg, mABg, mAWg, mTBg, and mTWg, and among which, vHg, vWg, vABg, vAWg, vTBg, and vTWg are preferred, or alternatively a combination of these and a parameter related to another centroid point or another parameter, such as vTg, vB, Vmax, Amax, Amin, VmaxT, AmaxT, and AminT is also preferred. When the parameter set includes two or more parameter groups of parameters related to a centroid point, each of the parameter groups is derived from parameters related to a different type of centroid points. For example, the parameter set can include a combination of parameter groups of different parameters related to a centroid point of a linear curve, a combination of parameter groups of different parameters related to a centroid point of a quadratic curve, or a combination of a parameter group of parameters related to a centroid point of a linear curve and a parameter group of parameters related to a centroid point of a quadratic curve. Preferable examples of the parameter set include a combination of parameter groups of vTg, vHg, vB, vABg, and vTBg, a combination of parameter groups of vB, vABg, and vTBg, and a combination of parameter groups of vB and vABg. Besides, a parameter set including such a combination of parameter groups, and Vmax, Amax, VmaxT, and AmaxT is also preferred.

The calculation target threshold used for obtaining parameters included in the parameter group may be in a range of 0% or more and less than 100%, and is preferably from 0.5 to 99.5%, more preferably from 5 to 95%, and further preferably from 5 to 90%. The number of calculation target regions used for one parameter may be 2 or more, and is preferably 5 or more, more preferably 10 or more, and is, for example, from 5 to 100, preferably from 5 to 50, and is, for example, from 5 to 20 or from 10 to 50.

For example, when L calculation target regions are extracted and a parameter to be employed is vHgx, the parameter sets include L vHgx. For example, 10 calculation target regions based on 10 calculation target thresholds (of 5%, 10%, 20%, 30%, 40%, 50%, 60%, 70%, 80%, and 90%) are extracted, and the parameter vHgx is calculated from each of the calculation target regions, the parameter set is a set of 10 vHgx [vHg5%, vHg10%, vHg20%, vHg30%, vHg40%, vHg50%, vHg60%, vHg70%, vHg80%, and vHg90%]. Similarly, when M calculation target regions are extracted, and parameters to be employed are vABgx and vBx, the parameter set includes M sets of [vABgx, vBx].

### 2.2.2. Template Specimen

In the present embodiment, the above-described parameter set of a test specimen (hereinafter, also referred to as the test parameter set) is compared with a corresponding parameter set of a template specimen (hereinafter, also referred to as the template parameter set). Based on a result of the comparison, the coagulation properties of the test specimen can be evaluated. In the present embodiment, one or more template specimens are prepared. The template specimen is a blood specimen having a known coagulation property (such as a coagulation time elongation factor, the presence or degree of a blood coagulation abnormality, or a coagulation factor concentration) to be evaluated.

For example, when evaluation is to be made for FVIII, the one or more template specimens include one or more specimens having FVIII activity level not abnormal (FVIII normal specimens), and one or more specimens having abnormal FVIII activity level (FVIII abnormal specimens, such as FVIII deficient specimens). For example, when evaluation is to be made for FIX, the one or more template specimens include one or more specimens having FIX activity level not abnormal (FIX normal specimens), and one or more specimens having abnormal FIX activity level (FIX abnormal specimens, such as FIX deficient specimens). For example, when evaluation is to be made for FVIII and FIX, the one or more template specimens include one or more specimens having both FVIII and FIX activity levels not abnormal (FXIII/FIX normal specimens), one or more specimens having abnormal FVIII activity level (FVIII abnormal specimens, such as FVIII deficient specimens), and one or more specimens having abnormal FIX activity level (FIX abnormal specimens, such as FIX deficient specimens).

The FVIII abnormal specimens preferably include specimens derived from serious, moderate and mild hemophilia A patients. The specimens derived from serious, moderate and mild hemophilia A patients are preferably specimens having FVIII activity of respectively less than 1%, 1% or more and less than 5%, and 5% or more and less than 40% (which values are obtained assuming that activity in a normal person is 100%; which also applies to values mentioned below). When evaluation in more detail is required, a plurality of specimens derived from serious hemophilia A patients having different FVIII activity levels may be prepared if necessary. For example, specimens derived from modestly-severe hemophilia A (MS-HA) patients having FVIII activity of 0.2% or more and less than 1%, and specimens derived from very-severe hemophilia A (VS-HA) patients having FVIII activity of less than 0.2% may be prepared. In recent years, it has been reported that there is a difference in clinical severity between VS-HA patients having particularly low FVIII activity (having FVIII activity of less than 0.2%) and MS-HA patients not having the low activity (having FVIII activity of 0.2% or more and less than 1%) among serious hemophilia A patients (Tomoko Matsumoto, Midori Shima, Clot Waveform Analysis and its Application to the Detection of Very Low Levels of Factor VIII Activity, 2003, vol. 14, No. 2, pp. 122-127). It is useful for offering appropriate treatment to a patient to discriminate a VS-HA patient.

Similarly, the FIX abnormal specimens preferably include specimens derived from serious, moderate, and mild hemophilia B patients. The specimens derived from serious, moderate, and mild hemophilia B patients are preferably specimens having FIX activity of respectively less than 1%, 1% or more and less than 5%, and 5% or more and less than 40% (which values are obtained assuming that activity in a normal person is 100%; which also applies to values mentioned below). When evaluation in more detail is to be performed, a plurality of specimens derived from serious hemophilia B patients having different FIX activity levels may be prepared if necessary. For example, a specimen having FIX activity of 0.2% or more and less than 1%, and a specimen having FIX activity of less than 0.2% may be prepared.

### 2.2.3. Comparison With Template

In the present embodiment, a test parameter set is compared with each of template parameter sets derived from template specimens. Preferably, regression analysis is performed between the test parameter set and each of the template parameter sets.

Each template parameter set used in regression analysis includes parameters corresponding to the test parameter set. In other words, the type of parameters included in the template parameter set and a series of calculation target thresholds used for calculating these are the same as those of the test parameter set. Respective parameters included in each template parameter set mutually correspond to respective parameters included in the test parameter set. For example, when the test parameter set includes L vHgx ( [vHgx₁, vHgx₂, ..., and vHgx_{L}]), the template parameter set also includes L vHgx ([vHgx₁, vHgx₂, ..., and vHgx_{L}] ).

The template parameter set is desirable to be precedently obtained. Besides, each template parameter set may be a synthetic parameter set obtained by processing parameter sets obtained from a plurality of template specimens. For example, a parameter set of a plurality of template specimens having similar coagulation properties is obtained, and subjected to statistical processing, and thus, one or more synthetic parameter sets corresponding to standard template specimens may be created.

A method of the regression analysis is not especially limited, and an example includes least squares linear regression. For example, a regression line is obtained assuming that a value of each parameter of the test parameter set is y, and that a value of a corresponding parameter in any one of the template parameter sets is x. Based on the slope, an intercept, and correlation (such as a correlation coefficient, or a coefficient of determination) or the like of the regression line, correlation between the test parameter set and each template parameter set is examined. The correlation between the test parameter set and the template parameter set reflects correlation (approximate state) in coagulation properties between the test specimen and the template specimen from which the template parameter set is derived.

### 2.2.4. Evaluation of Coagulation Properties

Next, based on a result of the regression analysis, coagulation properties of the test specimen are evaluated. Examples of the coagulation properties to be evaluated include a coagulation time elongation factor, the presence or degree of blood coagulation abnormality, or a coagulation factor concentration, and it is preferably the presence or degree of blood coagulation abnormality, or a coagulation factor concentration (activity level). Examples of the blood coagulation abnormality to be evaluated include hemophilia A and hemophilia B, and it is preferably hemophilia A. Examples of the type of the coagulation factor to be evaluated include one or more selected from the group consisting of FV, FVIII, FIX, FX, FXI, and FXII, among which FVIII or FIX is preferred, and FVIII is more preferred. Hereinafter, procedures for determining FVIII activity level or activity abnormality (hemophilia A) will be described as an example. Evaluation may be made in a similar manner for the other factors such as FIX.

### 2.2.4.1. Evaluation of FVIII Activity Level

The template specimens include one or more FVIII normal specimens, and one or more FVIII abnormal specimens variously different in FVIII activity level. Preferably, the template specimens include one or more FVIII normal specimens, and one or more FVIII abnormal specimens derived from each of serious (VS-HA and MS-HA if necessary), moderate, and mild hemophilia A patients. From all the template specimens used in the regression analysis, at least one specimen which meets the prescribed criteria in the correlation between the test parameter set and the template parameter set is selected.

In one embodiment, a template specimen having the correlation equal to or larger than a precedently set threshold is selected. In another embodiment, a template specimen having the correlation (for example, a correlation coefficient) equal to or larger than a prescribed value, and having the highest correlation is selected. In another embodiment, a template specimen having a slope of the regression line between the test parameter set and the template parameter set falling in a prescribed range (for example, 0.70 or more and 1.30 or less, preferably 0.75 or more and 1.25 or less, more preferably 0.80 or more and 1.20 or less, further preferably 0.85 or more and 1.15 or less, and still further preferably 0.87 or more and 1.13 or less) is selected. In another embodiment, a template specimen having the slope of the regression line between the test parameter set and the template parameter set falling in the prescribed range, and having a correlation coefficient of the regression line of equal to or larger than a prescribed value (for example, larger than 0.75, preferably larger than 0.80, more preferably larger than 0.85, and further preferably larger than 0.90) is selected. On the other hand, when a template specimen which meets the prescribed criteria is not selected, the criteria may be changed to select a template specimen again, or evaluation is made as “no template specimen selected.”

In one preferable embodiment, a template specimen having the slope of the regression line falling in a prescribed range (for example, 0.70 or more and 1.30 or less, preferably 0.75 or more and 1.25 or less, more preferably 0.80 or more and 1.20 or less, further preferably 0.85 or more and 1.15 or less, and still further preferably 0.87 or more and 1.13 or less) is selected. Preferably, a template specimen having the slope of the regression line falling in the prescribed range, and having a correlation coefficient of the regression line equal to or larger than a prescribed value (for example, larger than 0.75, preferably larger than 0.80, more preferably larger than 0.85, and further preferably larger than 0.90) is selected. From template specimens thus selected, a template specimen having the largest correlation coefficient of the regression is selected. When a plurality of template specimens meeting the prescribed criteria are selected, one template specimen may be selected therefrom based on another criterion.

Next, a state of FVIII (namely, FVIII activity level or activity abnormality) in the selected template specimen is determined as the state of FVIII in the test specimen. When a plurality of template specimens are selected, the state of FVIII in the test specimen may be determined to correspond to the state in any of the plurality of template specimens, or an average state in the plurality of template specimens may be determined as the state of FVIII in the test specimen.

For example, when the selected template specimen is an FVIII normal specimen, the state of FVIII in the test specimen can be determined to have no abnormality, and on the other hand, when the selected template specimen is an FVIII abnormal specimen, the test specimen can be determined to have abnormality of FVIII activity. In addition, for example, when the selected template specimen is a specimen derived from a serious, moderate, or mild hemophilia A patient, the test specimen can be determined to have serious, moderate or mild hemophilia A. Besides, for example, when the selected template specimen is a specimen derived from a VS-HA or MS-HA patient, the test specimen can be determined as VS-HA or MS-HA. Alternatively, when the FVIII activity level in the test specimen is to be determined, the FVIII activity level of the selected template specimen can be determined as the FVIII activity level of the test specimen.

On the other hand, when the template specimens include specimens derived from serious, moderate, and mild hemophilia A patients, and the correlation is evaluated as “no template specimen selected” as described above, the test specimen can be determined to “have no abnormality of FVIII activity,” or the test specimen can be determined that the “blood coagulation time elongation factor is not caused by abnormality of FVIII activity.”

In another preferable embodiment, template specimens having the slope of the regression line falling in a prescribed range (for example, 0.70 or more and 1.30 or less, preferably 0.75 or more and 1.25 or less, more preferably 0.80 or more and 1.20 or less, further preferably 0.85 or more and 1.15 or less, and still further preferably 0.87 or more and 1.13 or less) are all selected. Preferably, template specimens having the slope of the regression line falling in the prescribed range, and having a correlation coefficient of the regression line equal to or larger than a prescribed value (for example, larger than 0.75, preferably larger than 0.80, more preferably larger than 0.85, and further preferably larger than 0.90) are all selected. A state of FVII (namely, FVIII activity level or activity abnormality) found most frequently among the selected template specimens is determined as the state of FVIII in the test specimen.

For example, when the number of FVIII normal specimens is the largest among the selected template specimens, the state of FVIII in the test specimen can be determined to have no abnormality. On the other hand, when the number of FVIII abnormal specimens is the largest among the selected template specimens, the test specimen can be determined to have abnormality of FVIII activity. In addition, for example, when the number of specimens derived from serious, moderate, or mild hemophilia A patients is the largest among the selected template specimens, the test specimen each can be determined to have serious, moderate, or mild hemophilia A. For example, when the number of specimens derived from VS-HA or MS-HA serious hemophilia A patents is the largest among the selected template specimens, the test specimen each can be determined as VS-HA or MS-HA. For example, when the number of specimens having blood coagulation time elongation not corresponding to FVIII abnormality is the largest among the selected template specimens, the test specimen can be determined as an abnormal specimen but not of a (serious, moderate, or mild) hemophilia A patient. Alternatively, when the FVIII activity level in the test specimen is to be determined, FVIII activity level most frequently found in the selected template specimens can be determined as the FVIII activity level in the test specimen.

In another preferable embodiment, template specimens having the slope of the regression line falling in a prescribed range (for example, 0.70 or more and 1.30 or less, preferably 0.75 or more and 1.25 or less, more preferably 0.80 or more and 1.20 or less, further preferably 0.85 or more and 1.15 or less, and still further preferably 0.87 or more and 1.13 or less) are all selected. Preferably, template specimens having the slope of the regression line falling in the prescribed range, and having a correlation coefficient of the regression line equal to or larger than a prescribed value (for example, larger than 0.75, preferably larger than 0.80, more preferably larger than 0.85, and further preferably larger than 0.90) are all selected.

The selected template specimens are divided, in accordance with the FVIII activity level, into specimens having low FVIII activity and derived from (serious, moderate, or mild) hemophilia A patients, and the other specimens. When the number of the former specimens is larger than the number of the latter specimens, the severity (any one of serious, moderate, and mild severity) most frequently found in the former specimens is determined as the state of the test specimen. When there are the same number of specimens respectively having different severities, a more serious state may be determined as the state of the test specimen, or the criterion may be changed to select template specimens again. On the other hand, when the number of latter specimens is larger than the number of the former specimens, it is determined that the test specimen is not derived from a (serious, moderate, or mild) hemophilia A patient.

Through these procedures, the FVIII activity level, or presence of the activity abnormality in the test specimen can be determined. In one embodiment, the presence of the FVIII activity abnormality in the test specimen is determined, and this determination provides information on determination whether or not the test specimen is a specimen of a hemophilia A patient. In one embodiment, the FVIII activity level in the test specimen is determined, and this determination provides information on determination of hemophilia A severity of a patient having provided the test specimen. Accordingly, the method of the present embodiment can be a method for determining hemophilia A, and for determining severity of hemophilia A, for example, whether it is serious (VS-HA or MS-HA if necessary), moderate, or mild, or a method for acquiring data for such determination.

### 2.2.4.2. Evaluation of Activity Level of Other Coagulation Factors

In one embodiment, the test specimen may be evaluated for another coagulation factor. Preferably, the another coagulation factor is FIX. Evaluation of FIX activity level, or presence of the activity abnormality can be performed through similar procedures to those described above for evaluating FVIII activity level. In one embodiment, the presence of FIX activity abnormality in the test specimen is determined, and this determination provides information on determination whether or not the test specimen is a specimen of a hemophilia B patient. In another embodiment, FIX activity level in the test specimen is determined, and this determination provides information on severity determination of hemophilia B in the patient having provided the test specimen. The present embodiment enables the determination of hemophilia B, and determination of severity (for example, serious, moderate, or mild severity) of hemophilia B.

The evaluation of FIX may be performed separately from, or in combination with the evaluation of FVIII described above. When the evaluation of FVIII and the evaluation of FIX are combined, the coagulation properties of the test specimen can be more comprehensively analyzed. For example, a test specimen having been determined, in the evaluation of FVIII, that “the blood coagulation time elongation factor is not caused by FVIII activity abnormality” or as “not a (serious, moderate, or mild) hemophilia A patient” may be evaluated for FIX activity level, or presence of the activity abnormality. In this case, template specimens used in the evaluation of FIX may be the same as or different from those used in the evaluation of FVIII. The test parameter set and the template parameter set used in the evaluation of FIX may be the same as or different from those used in the evaluation of FVIII.

### 2.3. Evaluation of Coagulation Time Elongation Factor

In one embodiment, in the method of the present invention, a mixed specimen of a test specimen and a normal specimen is subjected to a heat treatment, and then a parameter related to the centroid point is compared between the heated specimen and an unheated specimen to evaluate a coagulation time elongation factor of the test specimen. For example, the heat treatment of the mixed specimen may affect the parameter related to the centroid point depending on the type of the elongation factor. Accordingly, when the parameter related to the centroid point is compared between the heated specimen and the unheated specimen, the elongation factor can be evaluated.

### 2.3.1 Specimen Preparation

An example of the test specimen to be analyzed in the present embodiment includes a blood specimen found to show coagulation time (such as APTT) elongation in a blood coagulation test.

In the present embodiment, a mixed specimen of the test specimen and a normal specimen is used in coagulation reaction measurement. For preparing the mixed specimen, the test specimen and a separately prepared normal specimen are mixed in a prescribed ratio. As the normal specimen, a blood specimen not showing coagulation time elongation is used. A commercially available normal specimen may be used. A mixing ratio between the test specimen and the normal specimen may be, in terms of a volume ratio assuming that a total volume is 10, test specimen:normal specimen of a range from 1:9 to 9:1, and is preferably a range from 4:6 to 6:4, and more preferably 5:5.

A part of the thus prepared mixed specimen is heated. A temperature of the heating may be, for example, 30° C. or more and 40° C. or less, and is preferably 35° C. or more and 39° C. or less, and more preferably 37° C. Time of the heating may be, for example, in a range from 2 to 30 minutes, and is preferably from 5 to 30 minutes, and more preferably about 10 minutes. The heating time may be further longer, and is preferably within 1 hour, and is within 2 hours at most. Herein, the mixed specimen resulting from the heat treatment is also designated as the “heated specimen”. On the other hand, in the method of the present embodiment, a mixed specimen not having been subjected to the heat treatment is also used, and this specimen is herein also designated as the “unheated specimen.” It is noted that the “unheated specimen” may be subjected to a preliminary heat treatment for a specimen in usual coagulation reaction measurement, for example, heating at 30° C. or more and 40° C. or less for 1 minute or less, and in this case, the “heated specimen” may be subjected to the preliminary heat treatment in addition to the above-described heat treatment.

### 2.3.2. Acquisition of Parameter

Subsequently, the coagulation reaction measurement is performed on the heated specimen and the unheated specimen. Accordingly, in the method of the present embodiment, the coagulation reaction measurement can be performed on a part of the prepared mixed specimen after the heat treatment, and on the other part without performing the heat treatment. Procedures of the coagulation reaction measurement are the same as those described in the above-described section 1.2. The order of performing the coagulation reaction measurement on the heated specimen and the unheated specimen is not especially limited. For example, after heating a part of the mixed specimen, the heated specimen and the unheated specimen may be subjected to the coagulation reaction measurement, or after subjecting the unheated specimen to the coagulation reaction measurement, the heated specimen may be subjected to the coagulation reaction measurement.

Based on coagulation reaction data of the heated specimen and the unheated specimen obtained through the coagulation reaction measurement, a parameter related to the centroid point, and other parameters if necessary, are acquired in accordance with the description of the above-described section 1.3. Hereinafter, a parameter acquired from the unheated specimen is designated as the first parameter (or Pa), and a parameter acquired from the heated specimen is designated as the second parameter (or Pb). In the present embodiment described below, when values of parameters, such as a ratio or a difference therebetween, are described, the term “parameter” and the term “parameter value” are used in the same meaning. On the other hand, when a type of a parameter is described, the term “parameter” and the term “parameter type” are used in the same meaning.

### 2.3.3. Evaluation of Elongation Factor

When the elongation factor is an antiphospholipid antibody such as a lupus anticoagulant (LA), or coagulation factor deficiency, the coagulation time is not largely changed by the heat treatment of the mixed specimen, but when the elongation factor is a coagulation factor inhibitor, elongation of the coagulation time is detected in the heated specimen. Therefore, in the method of the present embodiment, the elongation factor of the test specimen contained in the mixed specimen is evaluated based on a ratio (Pb/Pa) or difference (Pb -Pa) between the first parameter and the second parameter. Preferably, the evaluation of the elongation factor of the present embodiment is evaluation for determining which of coagulation factor deficiency, LA positive, and inhibitor positive is the elongation factor. Preferably, an inhibitor to be evaluated in the present embodiment is an FVIII inhibitor. Preferably, a coagulation factor to be evaluated in the present embodiment is FVIII. Those skilled in the art can easily presume that similar results can be obtained even when the type of the inhibitor is one for another coagulation factor such as factor IX (FIX) or factor V (FV).

In one example, when a ratio (Pb/Pa) between the first parameter and the second parameter does not fall in a prescribed range including 1, the elongation factor is evaluated as an inhibitor, for example, the presence of an inhibitor, and when the ratio (Pb/Pa) between the first parameter and the second parameter falls in the prescribed range including 1, the elongation factor is evaluated not as an inhibitor, but as LA or a coagulation factor, for example, the presence of LA or coagulation factor deficiency. In another example, when a difference (Pb - Pa) between the first parameter and the second parameter does not fall in a prescribed range including 0, the elongation factor is evaluated as an inhibitor, and when the difference (Pb - Pa) between the first parameter and the second parameter falls in the prescribed range including 0, the elongation factor is evaluated not as an inhibitor but as LA or a coagulation factor. It is noted that the elongation factor can be affected not only by the presence of an inhibitor or LA in a specimen or coagulation factor deficiency, but also by the amount thereof.

The first and second parameters used in the evaluation may be any one or more of the parameters related to the centroid point described above, or may be a combination of the parameter related to the centroid point and the other parameters described above. Alternatively, the first and second parameters may be resultant values of four arithmetic operations of the parameters related to the centroid point, or resultant values of four arithmetic operations of the parameters related to the centroid point and the other parameters. The first and second parameters are preferably one or more selected from the group consisting of vHg, vABg, vAWg, vTWg, pHg, pABg, pAWg, and mHg, and more preferably one or more selected from the group consisting of vABg, vAWg, and pHg. The first and second parameters are preferably parameters obtained from an uncorrected linear or quadratic curve. A calculation target threshold used for obtaining the parameters may be in a range of 0% or more and less than 100%, and is preferably from 0.5 to 99.5%, more preferably from 5 to 90%, and further preferably from 5 to 60%.

Discrimination between coagulation factor deficiency and LA positive can be conducted by comparing the first parameter (Pa) or the second parameter (Pb) itself. For example, Pa or Pb of a mixed specimen derived from a coagulation factor deficient specimen is a similar value to that of a normal specimen, and can be different from Pa or Pb of a mixed specimen derived from an LA positive specimen. Accordingly, it can be evaluated, based on the value of Pa or Pb of the mixed specimen, whether the elongation factor is coagulation factor deficiency or LA. Therefore, an example of the method of the present embodiment is a method for evaluating whether the elongation factor is an inhibitor, or LA or coagulation factor deficiency. Another example of the method of the present embodiment is a method for evaluating whether the elongation factor is LA or coagulation factor deficiency.

According to the present embodiment, the elongation factor can be quantitatively evaluated by using an index of a ratio or a difference between evaluation parameters. According to the present embodiment, a test time of the conventional cross-mixing test can be shortened. For example, as compared with the conventional cross-mixing test, the heating time for a specimen can be shortened.

### 2.4 Measurement of Coagulation Factor Inhibitor Titer

In one embodiment, in the method of the present invention, a mixed specimen of a test specimen and a normal specimen is subjected to a heat treatment, and a parameter related to the centroid point is compared between the heated specimen and an unheated specimen, and thus, a coagulation factor inhibitor titer of the test specimen is measured. For example, influence of the heat treatment of the mixed specimen on the parameter related to the centroid point may be changed in accordance with the titer of a coagulation factor inhibitor in some cases. Accordingly, when the parameter related to the centroid point is compared between the heated specimen and the unheated specimen, the titer of the coagulation factor inhibitor can be measured.

### 2.4.1. Specimen Preparation

An example of the test specimen to be analyzed in the present embodiment includes a blood specimen found to show coagulation time (such as APTT) elongation in a blood coagulation test, and is preferably a blood specimen which shows coagulation time elongation due to the presence of a coagulation factor inhibitor. More preferably, the test specimen is a specimen which has been confirmed to show coagulation time elongation due to the presence of an inhibitor by a cross-mixing test or the like, and in which the type of the coagulation factor inhibited by the inhibitor has been identified by a coagulation factor activity test.

In the present embodiment, a heated specimen and an unheated specimen prepared from a mixed specimen of a test specimen and a normal specimen are used in the coagulation time measurement. The mixed specimen can be prepared in accordance with procedures similar to those described above in the section 2.3.1. A mixing ratio between the test specimen and the normal specimen may be, in terms of a volume ratio assuming that a total volume is 10, test specimen:normal specimen of a range from 1:9 to 9:1, and is preferably a range from 4:6 to 6:4, and more preferably 5:5. When an inhibitor titer of the test specimen is high, the test specimen may be precedently diluted about 2 to 100 times before mixing with the normal specimen, and the resultant diluted specimen may be mixed with the normal specimen in the above-described volume ratio to prepare the mixed specimen. For the dilution of the test specimen, normal plasma, a buffer, FVIII deficient plasma or the like can be used. Alternatively, a mixed specimen containing the test specimen and the normal specimen in the above-described volume ratio may be diluted with the normal specimen to a final volume ratio of the test specimen of about ½ to 1/100 to prepare a diluted mixed specimen. A heated specimen and an unheated specimen are prepared from the mixed specimen. The procedures for preparing the heated specimen and the unheated specimen are the same as those described above in the section 2.3.1.

### 2.4.2. Acquisition of Parameter

The procedures for the coagulation reaction measurement of the heated specimen and the unheated specimen are the same as those described above in the section 1.2. From coagulation reaction data of the heated specimen and the unheated specimen obtained through the coagulation reaction measurement, a parameter related to the centroid point, and other parameters if necessary, are acquired in accordance with the section 1.3. A parameter acquired from the unheated specimen is designated as the first parameter (or Pa), and a parameter acquired from the heated specimen is designated as the second parameter (or Pb). In the present embodiment described below, when values of parameters, such as a ratio or a difference therebetween, are described, the term “parameter” and the term “parameter value” are used in the same meaning. On the other hand, when a type of a parameter is described, the term “parameter” and the term “parameter type” are used in the same meaning.

### 2.4.3. Measurement of Inhibitor Titer

In one embodiment, it has been determined that a test specimen contained in a mixed specimen is a specimen showing coagulation time elongation due to the presence of a specific inhibitor. In this case, the inhibitor titer can be calculated based on the first and second parameters in accordance with procedures described below. In another embodiment, it is unknown whether or not a test specimen contained in a mixed specimen shows coagulation time elongation due to the presence of a specific inhibitor. In this case, after conducting evaluation of the elongation factor or identification of the type of an inhibitor, the inhibitor titer can be calculated based on the first and second parameters in accordance with procedures described below. The evaluation of the elongation factor and the identification of the type of the inhibitor may be conducted in accordance with the conventional cross-mixing test or coagulation factor activity test, or may be conducted in accordance with the method of the present invention described above in the sections 2.3.3. and 2.2. In the latter case, there is no need to conduct the time consuming convention cross-mixing test and coagulation factor activity test, and hence the measurement of the inhibitor titer is more simply realized.

In the method of the present invention, examples of an inhibitor to be measured for the titer are not especially limited, and include an FVIII inhibitor and an FIX inhibitor. Preferably, in the method of the present invention, the inhibitor titer is calculated in Bethesda equivalent unit (BU/mL).

In a mixed specimen containing a test specimen showing the coagulation time elongation due to a coagulation factor inhibitor, the shape of the coagulation reaction curve is changed by the heat treatment. Besides, the magnitude of the change in the shape of the coagulation reaction curve in the heated specimen is dependent on the activity (titer) of the inhibitor. As a result, the parameter values can be different between the heated specimen and the unheated specimen depending on the inhibitor titer. In the method of the present embodiment, the inhibitor titer of the test specimen contained in the mixed specimen is measured based on a ratio (Pb/Pa) or a difference (Pb - Pa) between the first parameter and the second parameter.

More specifically, a ratio or a difference between the first parameter and the second parameter is obtained from a mixed specimen containing a test specimen. By using a calibration curve of the target inhibitor titer, the target inhibitor titer can be calculated based on the ratio or difference between the first and second parameters. The calibration curve can be precedently created. For example, a series of specimens having known and variously different titers are used as standard specimens to prepare mixed specimens through the above-described procedures, the first parameters and the second parameters are obtained, and subsequently, a calibration curve may be created based on the inhibitor titers of the standard specimens and a ratio or a difference between the first and second parameters.

The first and second parameters used in the evaluation may be any one or more of the parameters related to the centroid point described above, or may be a combination of the parameter related to the centroid point and the other parameters described above. Alternatively, the first and second parameters may be resultant values of four arithmetic operations of the parameters related to the centroid point, or resultant values of four arithmetic operations of the parameters related to the centroid point and the other parameters. The first and second parameters are preferably one or more selected from the group consisting of vHg, vABg, vAWg, and vTWg, and more preferably vHg. A calculation target threshold used for obtaining the parameter may be in a range of 0% or more and less than 100%, and is preferably from 0.5 to 99.5%, more preferably from 0.5 to 90%, further preferably from 1 to 70%, and still further preferably from 1 to 60%.

### 3. Automatic Analyzer

The blood analysis method of the present invention described above can be automatically performed with a computer program. Accordingly, one aspect of the present invention is a program for performing the blood analysis method of the present invention. In addition, a series of steps of the method of the present invention including preparation of a specimen and measurement of a coagulation time can be automatically performed with an automatic analyzer. Accordingly, one aspect of the present invention is an apparatus for performing the blood analysis method of the present invention.

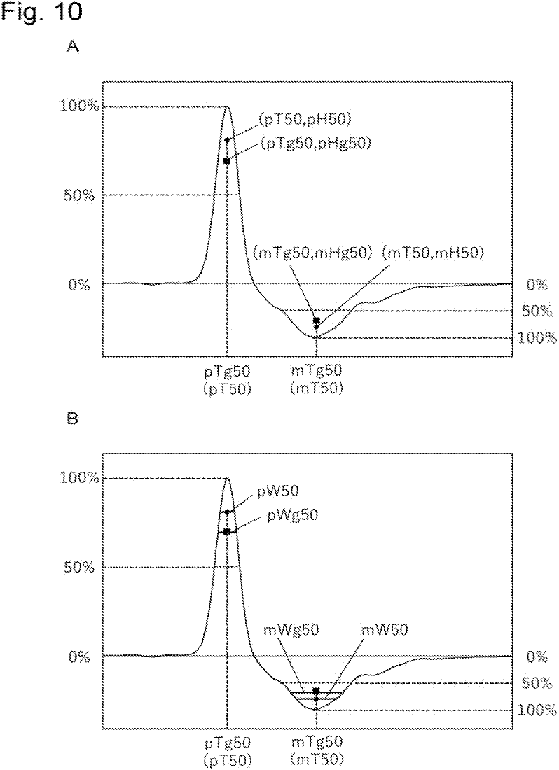

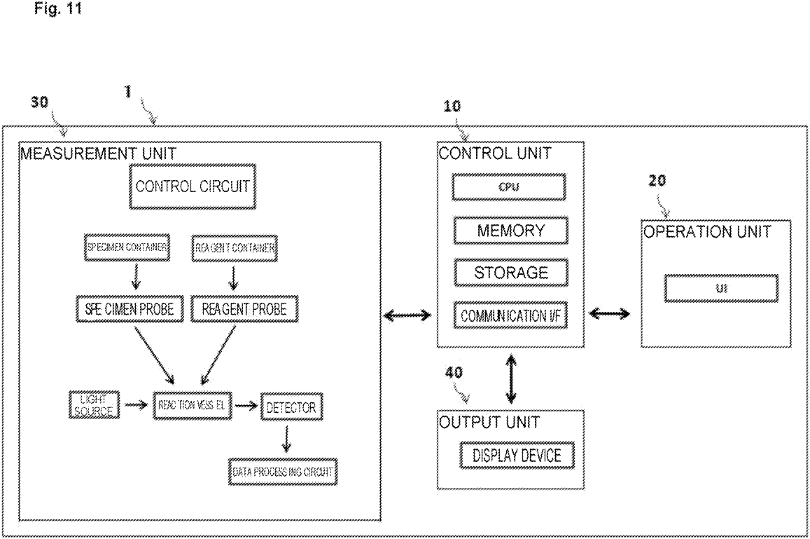

One embodiment of the apparatus of the present invention will now be described. One embodiment of the apparatus of the present invention is an automatic analyzer 1 as illustrated in FIG. 11. The automatic analyzer 1 includes a control unit 10, an operation unit 20, a measurement unit 30, and an output unit 40.

The control unit 10 controls the entire operation of the automatic analyzer 1. The control unit 10 can be constituted by, for example, one or more computers. The control unit 10 includes a CPU, a memory, a storage, a communication interface (I/F) and the like, and performs processing of commands from the operation unit 20, control of the operation of the measurement unit 30, storage of measurement data received from the measurement unit 30 and data analysis, storage of analysis results, control of output, by the output unit 40, of the measurement data and the analysis results, and the like. The control unit 10 may be further connected to another device such as an external medium, or a host computer. In the control unit 10, a computer for controlling the operation of the measurement unit 30 and a computer for analyzing the measurement data may be the same or different.

The operation unit 20 accepts input by an operator, and transmits input information thus obtained to the control unit 10. For example, the operation unit 20 includes a user interface (UI) such as a keyboard or a touch panel. The output unit 40 outputs, under control of the control unit 10, the measurement data of the measurement unit 30, and analysis results of the data. For example, the output unit 40 includes a display device such as a display.

The measurement unit 30 executes a series of operations for a blood coagulation test, and acquires measurement data of coagulation reaction of a sample including a blood specimen. The measurement unit 30 includes various devices and analysis modules necessary for the blood coagulation test, such as a specimen container for holding a blood specimen, a reagent container for holding a test reagent, a reaction vessel for causing a reaction between the specimen and the reagent, a probe for dispensing the blood specimen and the reagent into the reaction vessel, a detector for detecting scattered light or transmitted light from a light source and the specimen held in the reaction vessel, a data processing circuit for sending data from the detector to the control unit 10, a control circuit for controlling the operation of the measurement unit 30 in response to a command from the control unit 10, and the like.

The control unit 10 analyzes coagulation properties of the specimen based on the data measured with the measurement unit 30. This analysis can encompass acquisition of waveform data such as the above-described coagulation reaction curve, linear curve, and quadratic curve, calculation of a parameter of the specimen, evaluation of the coagulation properties (for example, determination of an elongation factor, and measurement of concentrations of respective components such as a coagulation factor and an inhibitor) based on the obtained parameter, and the like. The analysis can be executed by a program for performing the method of the present invention. Accordingly, the control unit 10 can include the program for performing the method of the present invention.

In the analysis performed in the control unit 10, the waveform data such as the coagulation reaction curve, the linear curve, and the quadratic curve may be created in the control unit 10 based on the measurement data from the measurement unit 30, or may be created in another device, for example, in the measurement unit 30 and sent to the control unit 10. Alternatively, the coagulation reaction curve may be created in the measurement unit 30 and sent to the control unit 10, so as to create the linear curve or the quadratic curve in the control unit 10. Information on a calibration curve and a template specimen, and data such as determination criteria for coagulation properties based on regression analysis with the template specimen may be precedently created in this device to be stored, or may be fetched from the outside. In accordance with the respective embodiments of the analysis method of the present invention, procedures for the analysis can be controlled by the program of the present invention.

The analysis result obtained in the control unit 10 is sent to the output unit 40 to be output. The output can be in an arbitrary form such as display on a screen, transmission to a host computer, or printing. Output information obtained from the output unit includes the evaluation result of the coagulation properties of the test specimen (such as an elongation factor, and concentrations of a coagulation factor and an inhibitor), and may further include, as desired, other information such as waveform data of the specimen, a parameter value, a calibration curve, information on the template specimen, and the result of the regression analysis. The type of the output information obtained from the output unit can be controlled by the program of the present invention.

In one embodiment, the automatic analyzer 1 can be in a structure as that of a general automatic analyzer for a blood coagulation test, such as those conventionally used in measurement of blood coagulation time such as APTT and PT except that the program for performing the method of the present invention is included.

### EXAMPLES

The present invention will now be described in detail by way of examples.

Parameters used in the following examples indicate parameters derived from corrected zero order to quadratic curves unless otherwise mentioned. On the other hand, parameters derived from uncorrected zero order to quadratic curves are indicated with a name of each parameter starting with R. For example, when a centroid height of a corrected linear curve is vHg, a centroid height of an uncorrected linear curve is indicated as RvHg. In the following description, a flattening and and a time rate may be represented by an expression with a coefficient k omitted in some cases.

A list of parameters related to a centroid point and other parameters used in blood analysis in the present examples are shown in the following Table 1.

TABLE 1

Parameters related to Centroid Point

| | | | | | |
|---|---|---|---|---|---|
| | | | Linear Curve | Quadratic Curve (positive peak) | Quadratic Curve (negative peak) |
| Centroid Height | | Hgx | vHgx | pHgx | mHgx |
| Centroid Time | | Tgx | vTgx | pTgx | mTgx |
| Centroid Peak Width | | Wgx | vWgx | pWgx | mWgx |
| B flattening | (Hgx/Bx)*k | | vABgx | pABgx | mABgx |
| W flattening | (Hgx/Wgx)*k | | vAWgx | pAWgx | mAWgx |
| W time rate | (Tgx/Wgx)*k | | vTWgx | pTWgx | mTWgx |

Other Parameters

| | | | | | |
|---|---|---|---|---|---|
| | | | Linear Curve | Quadratic Curve (positive peak) | Quadratic Curve (negative peak) |
| Peak Width (time) | | Bx | vBx | pBx | mBx |
| Weighted Average Height | | Hx | vHx | pHx | mHx |
| Weighted Average Time | | Tx | vTx | pTx | mTx |
| Weighted Average Peak Width | | Wx | vWx | pWx | mWx |
| B flattening | (Hx/Bx)*k | | vABx | pABx | mABx |
| W flattening | (Hx/Wx)*k | | vAWx | pAWx | mAWx |
| W time rate | (Tx/Wx)*k | | vTWx | pTWx | mTWx |

X: Calculation Target Threshold k: constant
A parameter derived from an uncorrected coagulation reaction curve has a name starting with R.

### Example 1 Relationship Between Coagulation Properties and Parameters

### 1) Preparation of Specimen

As a test specimen, FVIII deficient plasma (Factor VIII Deficient Plasma; manufactured by George King Bio-Medical, Inc., FVIII concentration regarded as 0%), or FIX deficient plasma (Factor IX Deficient Plasma; manufactured by George King Bio-Medical, Inc., FIX concentration regarded as 0%) was used. As a normal specimen, normal pooled plasma in which an FVIII concentration or an FIX concentration could be regarded as 100% was used. Each of the FVIII deficient plasma and the FIX deficient plasma was mixed with the normal pooled plasma in various volume ratios to prepare specimens respectively having a concentration of either of the factors of 50%, 25%, 10%, 5%, 2.5%, 1%, 0.75%, 0.5%, and 0.25% (N = 1 with respect to each concentration).

### 2) Coagulation Reaction Measurement

A coagulation reaction of each specimen was measured. The specimen was mixed with a coagulation time measuring reagent to prepare a sample, and photometric data on the amount of scattered light was acquired. As the measuring reagent, Coagpia APTT-N (manufactured by Sekisui Medical C., Ltd.), that is, an APTT measuring reagent, was used, and as a calcium chloride solution, Coagpia APTT-N calcium chloride solution (manufactured by Sekisui Medical Co., Ltd.) was used. The coagulation reaction measurement was performed with Blood Coagulation Automatic Analyzer CP3000 (manufactured by Sekisui Medical Co., Ltd.). To 50 µL of the sample having been discharged to a cuvette (reaction vessel) and heated at 37° C. for 45 seconds, 50 µL of the APTT measuring reagent having been heated to about 37° C. was added (discharged), and after elapse of 171 seconds, 50 µL of 25 mM calcium chloride solution was added (discharged) to start a coagulation reaction. The reaction was conducted with the temperature of 37° C. kept. The measurement (photometry) of the coagulation reaction was performed by emitting light from a light source of an LED with a wavelength of 660 nm, and detecting the amount of side scattered light at 90 degrees at intervals of 0.1 seconds. A measurement time was 360 seconds.

### 3) Data Analysis

The thus obtained coagulation reaction data was subjected to smoothing processing including denoising, and zero adjustment for making zero (0) the amount of scattered light at the start of photometry, and thus, a coagulation reaction curve (uncorrected zero order curve) was obtained. Subsequently, the coagulation reaction curve was corrected to obtain a maximum height of 100 to obtain a corrected coagulation reaction curve (corrected zero order curve). The corrected zero order curve thus obtained was first differentiated to obtain a corrected linear curve, which was further differentiated to obtain a corrected quadratic curve. Similarly, an uncorrected linear curve and an uncorrected quadratic curve were obtained from an uncorrected zero order curve.

### 4) Parameter Calculation

From the corrected linear curve, a maximum value (Vmax), and a centroid point (vTg, vHg) and a weighted average point (vT, vH) were calculated. A calculation target threshold for calculating the centroid point and the weighted average point was set to 0.5 to 95% of the maximum height Vmax (100%) of the linear curve.

### 5) Relationship Between Coagulation Factor Concentration and Parameter

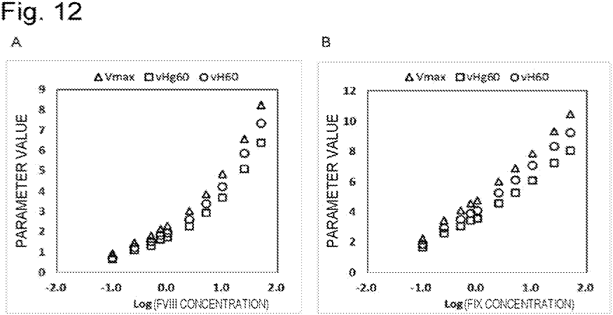

FIG. 12 illustrates a relationship between a coagulation factor concentration and a parameter. In FIG. 12, the maximum value Vmax (triangle), a centroid height vHg60% (square), and a weighted average height vH60% (circle) are plotted against a logarithm of an FVIII concentration (FIG. 12A) or an FIX concentration (FIG. 12B) (Log (FVIII concentration) or Log (FIX concentration)). When FVIII or FIX deficient plasma was logarithmically transformed, the calculation was performed assuming that the concentration was 0.1%. As is obvious from FIG. 12, vHg was highly correlated with the FVIII concentration and the FIX concentration. Accordingly, it was revealed that an FVIII concentration or an FIX concentration of a test specimen can be calculated from vHg of the test specimen by using a calibration curve based on vHg of a specimen having a known coagulation factor concentration. In addition, with respect to vABg and vAWg, which are parameters related to vHg, it was suggested that calibration curves can be similarly created, and that the FVIII concentration or the FIX concentration in the test specimen can be calculated by using the calibration curve.

FIGS. 13A to 13C illustrate corrected linear curves obtained from the normal specimen, the FVIII deficient plasma, and the FIX deficient plasma. In these drawings, black circles indicate, successively in the upward direction, centroid points (black squares) and weighted average points (black circles) obtained when the calculation target thresholds were respectively 1 to 95%. FIGS. 14A to 14C illustrate, in corrected linear curves obtained from the normal specimen, the FVIII deficient plasma, and the FIX deficient plasma of FIGS. 13A to 13C, centroid heights vHg (white circles) and weighted average point heights vH (black circles) obtained when the calculation target thresholds were respectively 1 to 95%. Left graphs in FIGS. 14A to 14C illustrate vHg and vH obtained when the calculation target thresholds were 1 to 95%, and right graphs illustrate vHg and vH obtained when the calculation target thresholds were 0.5 to 10%.

As illustrated in FIG. 13 and FIG. 14, it was clarified that vHg is largely different between the normal specimen and the coagulation factor deficient specimen. Accordingly, it was revealed that a coagulation factor deficiency state can be detected by comparing vHg of a test specimen with that of a normal specimen.

### 6) Comparison Between Centroid Point and Weighted Average Point

It has been found that a parameter related to a weighted average point can be used in blood analysis (Patent Literatures 6 and 7, PCT/JP2019/044943, PCT/JP2020/003796, and PCT/JP2020/017507). As illustrated in FIG. 13 and FIG. 14, when the calculation target threshold is the same, the centroid point and the weighted average point are in the same position on the abscissa but in different positions on the ordinate. Specifically, when the calculation target threshold was lower than 2%, vHg tended to be larger than vH, and when the calculation target threshold was higher than about 2%, vHg tended to be smaller than vH. As the calculation target threshold was increased, vHg linearly increased, but increment of vH gradually reduced. Therefore, a difference between vHg and vH gradually reduced as the calculation target threshold was increased. In the FVIII deficient plasma and the FIX deficient plasma (FIGS. 14B and 14C), the increment rate of vH was changed when the calculation target threshold was about 70°. This is probably because of, as found in FIGS. 13B and 13C, influence of the corrected linear curves of the FVIII deficient plasma and the FIX deficient plasma having bimodal peaks.

### Example 2 Measurement of Coagulation Factor Concentration

### 1) Preparation of Mixed Specimen

The FVIII deficient plasma and the normal specimen used in Example 1 were mixed in different ratios to prepare test specimens having different coagulation factor concentrations. The FVIII deficient plasma and the normal specimen used here were the same as those used in Example 1. The FVIII concentrations in the test specimens were prepared to be 50%, 25%, 10%, 5%, 2.5%, 1%, 0.75%, 0.5%, 0.25%, and 0.1% (only the FVIII deficient plasma) (N = 1 with respect to each concentration).

### 2) Coagulation Reaction Measurement and Data Analysis

The coagulation reaction measurement and the data analysis were performed through the same procedures as in Example 1.

### 3) Parameter Calculation

A coagulation time (APTT) was obtained from a resultant corrected zero order curve. The APTT was defined as a time (T50) when a 50% height was reached assuming that the maximum height of the corrected zero order curve was 100%. A maximum value (Vmax) was obtained from a corrected linear curve, and maximum and minimum second derivative values (Amax and Amin) were obtained from a corrected quadratic curve. A parameter related to the centroid point was calculated from the linear curve and the quadratic curve. A calculation target threshold used for calculating the centroid point was set in a range from 5 to 95% of Vmax, Amax, or Amin (100%). The same calculation target threshold was used to calculate a parameter related to the weighted average point.

### 4) Creation of Calibration Curve

With respect to each of the calculated parameters, a linear regression line of a logarithmically transformed parameter against a logarithm of the FVIII concentration (50%, 25%, 10%, 5%, 2.5%, 1%, 0.75%, 0.5%, or 0.25%) (Log (FVIII concentration)) in the specimen was obtained, which was used as a log-log calibration curve for the parameter.

### 5) Calculation of FVIII Concentration

Based on the calibration curve, the FVIII concentration in a specimen was calculated from each parameter. A ratio (%) of the calculated FVIII concentration to the actual concentration was evaluated as accuracy. When the actual concentration was 0%, the comparison was made with the case of the concentration of 0.1% (Log (FVIII concentration) = -1).

### 5.1) Parameter of Linear Curve

### 5.1.1) Centroid Time vTg

Table 2 shows a comparison ratio (%), to the actual concentration, of the FVIII concentration calculated based on the centroid time vTG and APTT (hereinafter sometimes referred to as the accuracy) in each calculation target region. In the table, cases having the accuracy within 100 ± 15% are grayed. With respect to the centroid time vTg (having the same value as the weighted average time), the accuracy was within 100 ± 15% at all the concentrations when the calculation target threshold was 80%. Besides, in the APTT and in the calculation target thresholds of vTg of from 5 to 60% excluding a concentration of 0°, the accuracy was within 100 ± 15%. It was thus suggested that vTg is a parameter related to the FVIII concentration equivalently to or more than the APTT.

TABLE 2

| | vTg5% | vTg10% | vTg20% | vTg30% | vTg40% | vTg50% | vTg60% | vTg70% | vTg80% | vTg90% | vTg95% | APTT |
|---|---|---|---|---|---|---|---|---|---|---|---|---|
| FVIII (0%) | 56.7 | 58.7 | 60.9 | 65.3 | 68.8 | 73.5 | 78.0 | 85.7 | 85.6 | >999 | >999 | 60.9 |
| FVIII (0.25%) | 110.4 | 109.7 | 110.9 | 112.2 | 113.3 | 113.5 | 113.1 | 116.8 | 111.6 | 124.1 | 126.3 | 111.0 |
| FVIII (0.5%) | 91.8 | 93.1 | 93.0 | 93.8 | 92.1 | 93.9 | 94.9 | 93.5 | 87.9 | 81.6 | 98.1 | 92.5 |
| FVIII (0.75%) | 107.5 | 107.7 | 106.9 | 106.4 | 106.5 | 106.9 | 107.0 | 105.6 | 113.2 | 118.8 | 100.2 | 108.0 |
| FVIII (1%) | 99.2 | 98.7 | 98.8 | 97.8 | 98.6 | 97.2 | 96.8 | 97.1 | 98.4 | 89.4 | 93.4 | 98.3 |
| FVIII (2.5%) | 92.3 | 92.3 | 91.8 | 91.4 | 91.7 | 90.9 | 91.0 | 89.9 | 94.6 | 86.0 | 83.7 | 91.9 |
| FVIII (5%) | 96.7 | 96.2 | 95.6 | 95.0 | 94.6 | 93.5 | 93.0 | 92.4 | 94.2 | 98.8 | 95.7 | 95.5 |
| FVIII (10%) | 94.9 | 94.4 | 94.3 | 93.8 | 93.7 | 93.3 | 92.8 | 92.1 | 92.3 | 98.9 | 82.0 | 94.1 |
| FVIII (25%) | 100.0 | 100.3 | 100.5 | 100.5 | 100.4 | 100.4 | 100.1 | 100.5 | 98.1 | 100.5 | 109.2 | 100.7 |
| FVIII (50%) | 109.3 | 109.7 | 110.3 | 111.6 | 111.9 | 113.4 | 114.4 | 116.1 | 113.4 | 110.0 | 120.2 | 110.2 |

### 5.1.2) Centroid Height vHg

Table 3A shows a comparison ratio (accuracy) (%), to the actual concentration, of the FVIII concentration calculated based on the centroid height vHg in each calculation target region in speciments respectively having different FVIII concentrations. In the table, cases having the accuracy within 100 ± 10% are grayed. For comparison, Table 3B s imi la rly shows accuracy obtained using the weighted average height vH. It was thus suggested that a calculated concentration based on vHg has higher accuracy than a calculated concentration based on vH, and that vHg is a parameter having higher correlation with the FVIII concentration than vH.

TABLE 3

A: vHg

| | vHg5% | vHg10% | vHg20% | vHg30% | vHg40% | vHg50% | vHg60% | vHg70% | vHg80% | vHg90% | vHg95% |
|---|---|---|---|---|---|---|---|---|---|---|---|
| FVIII (0%) | 51.6 | 52.8 | 54.9 | 56.2 | 57.1 | 57.7 | 58.4 | 59.4 | 60.8 | 65.7 | 64.9 |
| FVIII (0.25%) | 104.4 | 104.6 | 104.9 | 104.9 | 104.8 | 104.7 | 104.5 | 104.4 | 104.1 | 102.7 | 101.7 |
| FVIII (0.5%) | 91.1 | 91.6 | 91.9 | 92.3 | 92.7 | 93.0 | 93.4 | 94.0 | 94.7 | 94.2 | 94.6 |
| FVIII (0.75%) | 108.2 | 108.0 | 107.9 | 107.7 | 107.7 | 107.6 | 107.5 | 107.3 | 107.3 | 107.0 | 107.2 |
| FVIII (1%) | 100.3 | 100.1 | 100.1 | 100.0 | 100.0 | 100.0 | 100.1 | 100.2 | 100.4 | 101.4 | 101.2 |
| FVIII (2.5%) | 93.2 | 93.2 | 93.1 | 93.2 | 93.2 | 93.2 | 93.2 | 93.2 | 93.2 | 95.1 | 95.1 |
| FVIII (5%) | 102.4 | 101.6 | 101.0 | 100.6 | 100.3 | 100.0 | 99.8 | 99.4 | 98.6 | 99.0 | 99.4 |
| FVIII (10%) | 99.6 | 99.3 | 98.9 | 98.7 | 98.6 | 98.4 | 98.2 | 97.9 | 97.5 | 97.6 | 99.1 |
| FVIII (25%) | 103.6 | 103.5 | 103.3 | 103.1 | 102.9 | 102.8 | 102.6 | 102.6 | 102.5 | 101.4 | 100.6 |
| FVIII (50%) | 98.5 | 99.2 | 100.0 | 100.5 | 100.9 | 101.3 | 101.6 | 102.0 | 102.7 | 102.3 | 101.6 |

B: vH

| | vH5% | vH10% | vH20% | vH30% | vH40% | vH50% | vH60% | vH70% | vH80% | vH90% | vH95% |
|---|---|---|---|---|---|---|---|---|---|---|---|
| FVIII (0%) | 39.0 | 44.6 | 46.1 | 50.9 | 53.0 | 53.8 | 53.8 | 54.4 | 56.3 | 68.2 | 65.3 |
| FVIII (0.25%) | 100.5 | 93.5 | 99.9 | 103.3 | 104.6 | 104.2 | 104.2 | 106.9 | 106.0 | 104.0 | 102.8 |
| FVIII (0.5%) | 85.0 | 94.0 | 93.3 | 94.1 | 89.8 | 92.4 | 92.8 | 91.6 | 95.8 | 94.7 | 93.3 |
| FVIII (0.75%) | 110.7 | 112.1 | 106.4 | 106.4 | 106.8 | 107.8 | 108.2 | 107.2 | 105.0 | 107.4 | 107.7 |
| FVIII (1%) | 103.6 | 101.7 | 102.0 | 99.5 | 101.6 | 99.5 | 99.3 | 100.0 | 99.3 | 101.1 | 101.5 |
| FVIII (2.5%) | 93.8 | 96.5 | 94.9 | 93.4 | 94.7 | 93.4 | 93.5 | 92.4 | 91.3 | 94.0 | 95.1 |
| FVIII (5%) | 112.1 | 107.8 | 104.4 | 102.2 | 102.7 | 101.8 | 101.0 | 100.2 | 99.7 | 98.4 | 98.6 |
| FVIII (10%) | 105.4 | 100.5 | 101.4 | 99.9 | 99.8 | 99.3 | 99.0 | 97.9 | 97.9 | 94.2 | 98.8 |
| FVIII (25%) | 99.8 | 102.9 | 103.3 | 103.8 | 103.2 | 103.6 | 102.8 | 103.5 | 103.7 | 103.5 | 100.7 |
| FVIII (50%) | 92.3 | 92.7 | 95.2 | 98.2 | 97.9 | 98.9 | 100.1 | 101.5 | 102.2 | 103.8 | 102.2 |

### 5.1.3) Centroid Peak Width vWg

Table 4A shows a comparison ratio (accuracy) (%), to the actual concentration, of the FVIII concentration calculated based on the centroid peak width vWg in each calculation target region in specimens respectively having different FVIII concentrations. In the table, cases having the accuracy within 100 ± 10% are grayed. For comparison, Table 4B similarly shows the accuracy obtained using the weighted average peak width vW. It was suggested that a calculattedconcentrationbased on vWg has somewhat higher accuracy than a calculated concentration based on vW,and that vWg is a parameter having equivalent or higher correlation with the FVIII concentration as compared with vW.

TABLE 4

A: vWg

| | vWg5% | vWg10% | vWg20% | vWg30% | vWg40% | vWg50% | vWg60% | vWg70% | vWg80% | vWg90% | vWg95% |
|---|---|---|---|---|---|---|---|---|---|---|---|
| FVIII (0%) | 55.9 | 56.8 | 57.2 | 58.9 | 61.2 | 67.1 | 76.3 | 98.3 | 312.1 | >999 | >999 |
| FVIII (0.25%) | 108.7 | 111.1 | 111.1 | 110.3 | 110.3 | 114.8 | 109.3 | 111.4 | 102.3 | 41.8 | 3.6 |
| FVIII (0.5%) | 88.5 | 89.3 | 92.0 | 91.1 | 93.1 | 92.4 | 93.6 | 125.3 | 124.0 | 71.7 | 671.3 |
| FVIII (0.75%) | 107.5 | 106.4 | 107.5 | 108.3 | 107.8 | 103.9 | 107.4 | 104.3 | 106.7 | 124.6 | 69.3 |
| FVIII (1%) | 100.6 | 100.3 | 98.8 | 98.5 | 98.6 | 99.2 | 102.1 | 94.7 | 111.3 | 181.2 | 170.0 |
| FVIII (2.5%) | 92.3 | 90.9 | 90.1 | 91.0 | 90.2 | 89.9 | 89.2 | 73.2 | 107.9 | 263.6 | 375.3 |
| FVIII (5%) | 100.4 | 99.2 | 96.0 | 97.3 | 96.5 | 96.2 | 95.9 | 81.5 | 52.7 | 68.5 | 249.1 |
| FVIII (10%) | 99.7 | 98.1 | 96.3 | 97.9 | 95.6 | 93.2 | 92.8 | 86.8 | 69.0 | 278.1 | >999 |
| FVIII (25%) | 104.3 | 104.2 | 105.8 | 102.1 | 101.8 | 103.6 | 101.6 | 108.4 | 106.3 | 32.6 | 8.5 |
| FVIII (50%) | 99.8 | 102.5 | 104.7 | 105.4 | 108.1 | 109.5 | 110.5 | 129.1 | 159.2 | 90.2 | 39.0 |

B: vW

| | vW5% | vW10% | vW20% | vW30% | vW40% | vW50% | vW60% | vW70% | vW80% | vW90% | vW95% |
|---|---|---|---|---|---|---|---|---|---|---|---|
| FVIII (0%) | 49.8 | 51.0 | 53.2 | 61.8 | 59.9 | 53.5 | 48.5 | 50.7 | 195.6 | >999 | >999 |
| FVIII (0.25%) | 106.7 | 104.4 | 106.9 | 113.1 | 105.1 | 108.3 | 106.1 | 104.3 | 75.6 | 25.3 | 22.8 |
| FVIII (0.5%) | 89.8 | 91.4 | 93.0 | 92.6 | 94.9 | 119.5 | 106.6 | 92.8 | 113.5 | 171.2 | 521.9 |
| FVIII (0.75%) | 107.5 | 110.5 | 106.8 | 104.0 | 105.5 | 111.9 | 117.8 | 98.2 | 60.3 | 102.0 | 45.5 |
| FVIII (1%) | 100.1 | 99.3 | 101.2 | 100.0 | 101.7 | 97.4 | 99.4 | 109.7 | 126.8 | 146.7 | 98.7 |
| FVIII (2.5%) | 91.0 | 91.9 | 91.4 | 89.1 | 93.3 | 71.8 | 102.2 | 115.3 | 153.5 | 198.4 | 166.6 |
| FVIII (5%) | 104.3 | 101.8 | 98.7 | 97.6 | 97.9 | 81.6 | 65.5 | 112.7 | 90.1 | 151.9 | 83.3 |
| FVIII (10%) | 100.4 | 98.1 | 96.4 | 95.3 | 95.7 | 87.9 | 76.8 | 59.9 | 258.3 | 148.2 | 536.8 |
| FVIII (25%) | 103.7 | 105.2 | 102.8 | 102.8 | 101.3 | 108.9 | 109.9 | 94.1 | 50.6 | 53.6 | 70.8 |
| FVIII (50%) | 98.2 | 98.9 | 104.2 | 107.9 | 105.4 | 126.5 | 133.8 | 130.9 | 84.5 | 64.4 | 35.4 |

### 5.1.4) B Flattening vABg

Table 5A shows a comparison ratio (accuracy) (%), to the actual concentration, of the FVIII concentration calculated using the B flattening vABg based on the centroid point in each calculation target region in specimens respectively having different FVIII concentrations. In the table, cases having the accuracy within 100 ± 10% are grayed. For comparison, Table 5B similarly shows the accuracy obtained using the B flattening vAB based on the weighted average point. It was suggested that a calculated concentration based on vABg has somewhat higher accuracy than a calculated concentration based on vAB, and that vABg is a parameter having equivalent or higher correlation with the FVIII concentration as compared with vAB.

TABLE 5

A: vABg

| | vABg5 % | vABg10 % | vABg20 % | vABg30 % | vABg40 % | vABg50 % | vABg60 % | vABg70 % | vABg80 % | vABg90 % | vABg95 % |
|---|---|---|---|---|---|---|---|---|---|---|---|
| FVIII (0%) | 48.4 | 51.2 | 52.9 | 56.8 | 58.9 | 60.6 | 62.0 | 67.3 | 105.4 | 1,281.2.00 | 819.9 |
| FVIII (0.25%) | 104.3 | 101.4 | 104.3 | 106.3 | 106.8 | 106.0 | 105.3 | 108.7 | 106.8 | 80.5 | 55.8 |
| FVIII (0.5%) | 88.7 | 92.5 | 92.6 | 93.6 | 91.0 | 93.6 | 94.8 | 93.7 | 114.3 | 116.6 | 117.3 |
| FVIII (0.75%) | 108.5 | 109.0 | 106.4 | 106.0 | 106.3 | 107.1 | 107.2 | 105.4 | 94.4 | 96.8 | 105.9 |
| FVIII (1%) | 101.4 | 100.8 | 100.9 | 99.5 | 100.8 | 99.3 | 99.1 | 100.8 | 98.7 | 121.7 | 120.9 |
| FVIII (2.5%) | 92.5 | 93.8 | 93.2 | 92.5 | 93.6 | 92.7 | 93.2 | 91.7 | 86.6 | 122.0 | 137.4 |
| FVIII (5%) | 105.5 | 103.1 | 101.2 | 99.5 | 100.1 | 99.2 | 98.3 | 96.7 | 92.4 | 90.9 | 95.9 |
| FVIII (10%) | 101.8 | 99.4 | 99.6 | 98.5 | 98.4 | 97.8 | 97.4 | 95.5 | 94.9 | 72.1 | 173.7 |
| FVIII (25%) | 102.2 | 103.5 | 103.4 | 103.6 | 102.8 | 103.1 | 101.8 | 102.3 | 103.3 | 94.3 | 58.5 |
| FVIII (50%) | 96.8 | 97.6 | 99.3 | 101.4 | 101.3 | 102.3 | 103.9 | 106.7 | 112.0 | 119.9 | 89.0 |

B: vAB

| | vAB5% | vAB10% | vAB20% | vAB30% | vAB40% | vAB50% | vAB60% | vAB70% | vAB80% | vAB90% | vAB95% |
|---|---|---|---|---|---|---|---|---|---|---|---|
| FVIII (0%) | 42.0 | 47.0 | 48.5 | 54.1 | 56.8 | 58.5 | 59.5 | 64.4 | 100.8 | 1,294.2.00 | 820.7 |
| FVIII (0.25%) | 102.4 | 95.8 | 101.8 | 105.5 | 106.7 | 105.7 | 105.1 | 109.9 | 107.8 | 81.2 | 56.2 |
| FVIII (0.5%) | 85.6 | 93.7 | 93.3 | 94.5 | 89.6 | 93.2 | 94.4 | 92.5 | 114.8 | 116.8 | 116.2 |
| FVIII (0.75%) | 109.8 | 111.1 | 105.6 | 105.3 | 105.9 | 107.2 | 107.6 | 105.4 | 93.5 | 97.1 | 106.3 |
| FVIII (1%) | 103.1 | 101.6 | 101.9 | 99.2 | 101.6 | 99.1 | 98.7 | 100.7 | 98.2 | 121.4 | 121.0 |
| FVIII (2.5%) | 92.7 | 95.5 | 94.1 | 92.6 | 94.3 | 92.8 | 93.3 | 91.4 | 85.8 | 121.0 | 137.4 |
| FVIII (5%) | 110.5 | 106.2 | 102.9 | 100.3 | 101.3 | 100.1 | 98.9 | 97.1 | 93.0 | 90.6 | 95.4 |
| FVIII (10%) | 104.8 | 100.0 | 100.9 | 99.1 | 99.0 | 98.3 | 97.8 | 95.5 | 95.1 | 70.7 | 173.2 |
| FVIII (25%) | 100.2 | 103.2 | 103.4 | 104.0 | 103.0 | 103.6 | 101.9 | 102.7 | 103.9 | 95.4 | 58.6 |
| FVIII (50%) | 93.7 | 94.3 | 96.9 | 100.2 | 99.8 | 101.1 | 103.2 | 106.4 | 111.6 | 120.9 | 89.4 |

### 5.1.5) W Flattening vAWg

Table 6A shows a comparison ratio (accuracy) (%), to the actual concentration, of the FVIII concentration calculated using the W flattening vAWg based on the centroid point in each calculation target region in specimens respectively having different FVIII concentrations. In the table, cases having the accuracy within 100 ± 10% are grayed. For comparison, Table 6B similarly shows the accuracy obtained using the W flattening vAW based on the weighted average point. It was suggested that a calculated concentration based on vAWg has somewhat higher accuracy than a calculated concentration based on vAW, and that vAWg is a parameter having equivalent or higher correlation with the FVIII concentration as compared with vAW.

TABLE 6

A: vAWg

| | vAWg5 % | vAWg10 % | vAWg20 % | vAWg30 % | vAWg40 % | vAWg50 % | vAWg60 % | vAWg70 % | vAWg80 % | vAWg90 % | vAWg95 % |
|---|---|---|---|---|---|---|---|---|---|---|---|
| FVIII (0%) | 53.8 | 54.8 | 56.0 | 57.6 | 59.2 | 62.4 | 67.1 | 76.2 | 126.8 | 941.7 | 795.2 |
| FVIII (0.25%) | 106.6 | 107.9 | 108.0 | 107.7 | 107.6 | 109.8 | 107.0 | 107.8 | 103.3 | 73.6 | 36.2 |
| FVIII (0.5%) | 89.8 | 90.4 | 91.9 | 91.7 | 92.9 | 92.7 | 93.5 | 108.3 | 106.9 | 85.1 | 173.7 |
| FVIII (0.75%) | 107.8 | 107.2 | 107.7 | 108.0 | 107.7 | 105.7 | 107.4 | 105.8 | 107.0 | 113.2 | 93.6 |
| FVIII (1%) | 100.4 | 100.2 | 99.4 | 99.3 | 99.2 | 99.6 | 101.1 | 97.4 | 105.1 | 125.7 | 118.9 |
| FVIII (2.5%) | 92.7 | 92.0 | 91.5 | 92.1 | 91.6 | 91.5 | 91.1 | 82.7 | 99.6 | 138.8 | 145.6 |
| FVIII (5%) | 101.4 | 100.4 | 98.4 | 98.9 | 98.3 | 98.0 | 97.7 | 90.2 | 74.4 | 86.3 | 132.2 |
| FVIII (10%) | 99.7 | 98.7 | 97.6 | 98.3 | 97.0 | 95.7 | 95.3 | 92.3 | 83.5 | 144.0 | 210.4 |
| FVIII (25%) | 103.9 | 103.9 | 104.5 | 102.6 | 102.3 | 103.2 | 102.1 | 105.4 | 104.2 | 66.6 | 46.7 |
| FVIII (50%) | 99.1 | 100.9 | 102.4 | 103.0 | 104.6 | 105.5 | 106.1 | 114.5 | 125.0 | 97.6 | 75.5 |

B: vAW

| | vAW5% | vAW10% | vAW20% | vAW30% | vAW40% | vAW50% | vAW60% | vAW70% | vAW80% | vAW90% | vAW95% |
|---|---|---|---|---|---|---|---|---|---|---|---|
| FVIII (0%) | 44.2 | 47.8 | 49.7 | 56.3 | 56.4 | 53.6 | 51.2 | 52.7 | 98.2 | 993.6 | 748.0 |
| FVIII (0.25%) | 103.6 | 99.0 | 103.4 | 108.3 | 104.9 | 106.2 | 105.1 | 105.7 | 91.1 | 60.3 | 63.0 |
| FVIII (0.5%) | 87.4 | 92.6 | 93.1 | 93.3 | 92.4 | 104.9 | 99.1 | 92.1 | 103.3 | 119.0 | 163.5 |
| FVIII (0.75%) | 109.0 | 111.3 | 106.6 | 105.1 | 106.2 | 109.8 | 112.6 | 103.0 | 82.0 | 105.3 | 81.3 |
| FVIII (1%) | 101.8 | 100.5 | 101.6 | 99.8 | 101.7 | 98.5 | 99.3 | 104.4 | 110.7 | 116.7 | 100.6 |
| FVIII (2.5%) | 92.3 | 94.1 | 93.1 | 91.2 | 94.0 | 82.1 | 97.5 | 102.4 | 115.1 | 125.4 | 114.2 |
| FVIII (5%) | 108.0 | 104.7 | 101.4 | 99.8 | 100.2 | 91.3 | 82.3 | 105.8 | 95.3 | 116.4 | 93.3 |
| FVIII (10%) | 102.8 | 99.3 | 98.8 | 97.5 | 97.7 | 93.5 | 87.8 | 78.1 | 151.0 | 112.2 | 171.5 |
| FVIII (25%) | 101.8 | 104.1 | 103.0 | 103.3 | 102.2 | 106.2 | 106.1 | 99.0 | 75.2 | 80.3 | 89.8 |
| FVIII (50%) | 95.3 | 95.8 | 99.7 | 103.1 | 101.7 | 111.7 | 114.9 | 114.1 | 93.9 | 86.3 | 72.4 |

### 5.1.6) W Time Rate vTWg

Table 7A shows a comparison ratio (accuracy) (%), to the actual concentration, of the FVIII concentration calculated using the W time rate vTWg based on the centroid point in each calculation target region in specimens respectively having different FVIII concentrations. In the table, cases having the accuracy within 100 ± 15% are grayed. For comparison, Table 7B similarly shows the accuracy obtained using the W time rate vTW based on the weighted average point. It was suggested that a calculated concentration based on vTWg has somewhat higher accuracy than a calculated concentration based on vTW, and that vTWg is a parameter having equivalent or higher correlation with the FVIII concentration as compared with vTW.

TABLE 7

A: vTWg

| | vTWg5 % | vTWg10 % | vTWg20 % | vTWg30 % | vTWg40 % | vTWg50 % | vTWg60 % | vTWg70 % | vTWg80 % | vTWg90 % | vTWg95 % |
|---|---|---|---|---|---|---|---|---|---|---|---|
| FVIII (0%) | 54.9 | 54.4 | 53.0 | 52.3 | 53.6 | 60.7 | 74.7 | 117.4 | >999 | >999 | 0.0 |
| FVIII (0.25%) | 106.5 | 112.8 | 111.4 | 108.2 | 107.0 | 116.2 | 105.5 | 104.9 | 85.5 | 0.0 | >999 |
| FVIII (0.5%) | 84.5 | 84.7 | 90.7 | 88.0 | 94.3 | 90.9 | 92.2 | 182.3 | 252.3 | 0.0 | 0.2 |
| FVIII (0.75%) | 107.5 | 104.9 | 108.3 | 110.5 | 109.3 | 100.8 | 107.8 | 102.6 | 94.3 | >999 | 331.2 |
| FVIII (1%) | 102.4 | 102.3 | 98.8 | 99.4 | 98.5 | 101.4 | 108.0 | 91.7 | 143.8 | >999 | 13.4 |
| FVIII (2.5%) | 92.4 | 89.1 | 88.0 | 90.5 | 88.5 | 88.8 | 87.4 | 56.3 | 141.5 | >999 | 0.6 |
| FVIII (5%) | 105.4 | 103.1 | 96.4 | 100.0 | 98.8 | 99.3 | 99.1 | 69.4 | 15.9 | 0.0 | 4.3 |
| FVIII (10%) | 106.1 | 102.9 | 98.7 | 103.0 | 97.7 | 93.1 | 92.8 | 80.6 | 37.8 | >999 | 0.0 |
| FVIII (25%) | 109.9 | 109.3 | 112.6 | 104.0 | 103.4 | 107.1 | 103.2 | 119.5 | 125.5 | 0.0 | >999 |
| FVIII (50%) | 88.9 | 94.3 | 98.2 | 98.7 | 104.1 | 105.5 | 106.5 | 147.8 | 320.7 | 0.0 | >999 |

B: vTW

| | vTW5% | vTW10% | vTW20% | vTW30% | vTW40% | vTW50% | vTW60% | vTW70% | vTW80% | vTW90% | vTW95% |
|---|---|---|---|---|---|---|---|---|---|---|---|
| FVIII (0%) | 42.2 | 42.9 | 45.5 | 58.1 | 51.4 | 35.1 | 23.4 | 21.0 | >999 | >999 | 0.0 |
| FVIII (0.25%) | 102.1 | 98.2 | 102.3 | 114.2 | 96.9 | 101.7 | 96.2 | 86.2 | 34.4 | 0.0 | >999 |
| FVIII (0.5%) | 87.2 | 89.4 | 93.0 | 91.1 | 98.1 | 164.6 | 127.5 | 91.5 | 189.9 | >999 | 0.0 |
| FVIII (0.75%) | 107.5 | 114.1 | 106.7 | 101.4 | 104.6 | 118.9 | 136.7 | 87.1 | 16.9 | 18.2 | >999 |
| FVIII (1%) | 101.4 | 100.2 | 103.9 | 102.5 | 105.3 | 97.7 | 103.4 | 134.4 | 211.7 | >999 | 72.1 |
| FVIII (2.5%) | 89.4 | 91.3 | 91.0 | 86.6 | 95.1 | 52.5 | 122.1 | 175.0 | 407.9 | >999 | 3.3 |
| FVIII (5%) | 114.9 | 109.2 | 102.5 | 100.6 | 101.8 | 68.1 | 38.1 | 156.9 | 82.4 | >999 | 184.1 |
| FVIII (10%) | 107.8 | 102.9 | 99.0 | 97.0 | 97.8 | 81.1 | 57.3 | 29.2 | >999 | >999 | 0.0 |
| FVIII (25%) | 108.7 | 111.7 | 105.5 | 105.4 | 102.3 | 121.3 | 127.1 | 84.3 | 13.2 | 0.0 | 830.8 |
| FVIII (50%) | 85.6 | 87.0 | 97.4 | 103.8 | 98.7 | 146.3 | 170.4 | 160.0 | 46.6 | 0.1 | >999 |

### 5.2) Parameter of Quadratic Curve

### 5.2.1) Centroid Height pHg

Table 8A shows a comparison ratio (accuracy) (s), to the actual concentration, of the FVIII concentration calculated using the centroid height pHg in each calculation target region in specimens respectively having different FVIII concentrations. In the table, cases having the accuracy within 100 ± 10% are grayed. For comparison, Table 8B similarly shows the accuracy obtained theweighted average height pH. It was suggested that pHg is a parameter having equivalent or higher correlation with the FVIII concentration as compared wi th pH.

TABLE 8

A: pHg

| | pHg5% | pHg10% | pHg20% | pHg30% | pHg40% | pHg50% | pHg60% | pHg70% | pHg80% | pHg90% | pHg95% |
|---|---|---|---|---|---|---|---|---|---|---|---|
| FVIII (0%) | 438.4 | 101.0 | 96.8 | 93.3 | 92.0 | 92.0 | 91.6 | 90.6 | 88.9 | 88.1 | 89.1 |
| FVIII (0.25%) | 42.9 | 110.2 | 112.8 | 115.8 | 117.5 | 118.0 | 118.4 | 119.1 | 121.4 | 122.8 | 122.9 |
| FVIII (0.5%) | 245.1 | 87.1 | 87.3 | 87.3 | 86.7 | 86.2 | 85.7 | 85.1 | 83.9 | 83.0 | 82.9 |
| FVIII (0.75%) | 252.4 | 106.3 | 105.7 | 104.6 | 103.8 | 103.5 | 103.3 | 103.0 | 102.4 | 102.3 | 102.4 |
| FVIII (1%) | 21.3 | 96.5 | 94.3 | 92.7 | 92.3 | 92.4 | 92.5 | 92.6 | 92.3 | 92.1 | 92.1 |
| FVIII (2.5%) | 165.6 | 97.0 | 97.7 | 97.7 | 97.9 | 98.2 | 98.5 | 98.7 | 98.6 | 98.5 | 98.5 |
| FVIII (5%) | 143.4 | 102.0 | 100.0 | 99.0 | 98.6 | 98.5 | 98.6 | 98.9 | 99.0 | 99.1 | 99.2 |
| FVIII (10%) | 116.6 | 102.0 | 102.0 | 102.1 | 102.3 | 102.4 | 102.4 | 102.5 | 102.6 | 102.5 | 102.6 |
| FVIII (25%) | 89.8 | 102.5 | 102.8 | 103.1 | 103.2 | 103.1 | 103.0 | 103.0 | 103.1 | 103.2 | 103.2 |
| FVIII (50%) | 71.1 | 98.2 | 99.4 | 100.3 | 100.7 | 100.6 | 100.6 | 100.5 | 100.7 | 100.8 | 100.8 |

B: pH

| | pH5% | pH10% | pH20% | pH30% | pH40% | pH50% | pH60% | pH70% | pH80% | pH90% | pH95% |
|---|---|---|---|---|---|---|---|---|---|---|---|
| FVIII (0%) | 474.7 | 112.5 | 114.1 | 105.9 | 93.4 | 92.3 | 95.1 | 95.2 | 93.5 | 86.8 | 89.6 |
| FVIII (0.25%) | 39.6 | 109.4 | 106.4 | 105.1 | 114.5 | 116.6 | 115.9 | 115.1 | 115.1 | 122.6 | 122.5 |
| FVIII (0.5%) | 246.2 | 83.8 | 84.3 | 87.6 | 87.4 | 87.8 | 87.9 | 88.1 | 87.3 | 83.7 | 82.8 |
| FVIII (0.75%) | 260.0 | 107.9 | 108.4 | 110.4 | 106.0 | 104.2 | 104.0 | 103.6 | 103.6 | 101.8 | 102.1 |
| FVIII (1%) | 22.7 | 98.4 | 100.8 | 98.1 | 92.8 | 92.9 | 92.6 | 92.3 | 92.8 | 92.2 | 92.7 |
| FVIII (2.5%) | 155.4 | 92.0 | 96.6 | 97.4 | 96.5 | 96.9 | 98.6 | 99.8 | 100.0 | 98.3 | 98.4 |
| FVIII (5%) | 156.2 | 112.6 | 107.2 | 101.8 | 100.4 | 97.9 | 97.3 | 97.8 | 98.9 | 98.9 | 99.6 |
| FVIII (10%) | 119.7 | 103.5 | 101.9 | 102.4 | 101.8 | 101.1 | 102.3 | 102.5 | 101.8 | 102.5 | 102.9 |
| FVIII (25%) | 87.2 | 100.6 | 100.0 | 100.2 | 103.2 | 103.8 | 102.8 | 103.6 | 103.0 | 103.2 | 102.7 |
| FVIII (50%) | 68.5 | 95.3 | 96.7 | 98.4 | 99.8 | 101.3 | 101.0 | 99.6 | 99.8 | 101.0 | 100.7 |

### 5.2.2) B Flattening pABg

Table 9A shows a comparison ratio (accuracy) (%), to the actual concentration, of the FVIII concentration calculated using the B flattening pABg based on the centroid point in each calculation target region in specimens respectively having different FVIII concentrations. In the table, cases having the accuracy within 100 ± 10% are grayed. For comparison, Table 9B similarly shows the accuracy obtained using the B flattening pAB based on the weighted average point. It was suggested that pABg is a parameter equivalently correlated with the FVIII concentration to pAB.

TABLE 9

A: pABg

| | pABg5 % | pABg10 % | pABg20 % | pABg30 % | pABg40 % | pABg50 % | pABg60 % | pABg70 % | pABg80 % | pABg90 % | pABg95 % |
|---|---|---|---|---|---|---|---|---|---|---|---|
| FVIII (0%) | 384.7 | 115.5 | 111.5 | 96.7 | 77.9 | 76.1 | 80.7 | 78.9 | 69.6 | 38.3 | 165.6 |
| FVIII (0.25%) | 50.5 | 108.2 | 108.1 | 110.0 | 129.5 | 135.2 | 135.2 | 135.5 | 146.2 | 261.7 | 247.3 |
| FVIII (0.5%) | 202.6 | 82.3 | 83.1 | 87.3 | 85.4 | 84.7 | 83.2 | 81.2 | 74.5 | 50.3 | 43.5 |
| FVIII (0.75%) | 219.0 | 103.8 | 103.6 | 104.5 | 96.8 | 93.3 | 92.1 | 90.2 | 87.8 | 75.0 | 78.4 |
| FVIII (1%) | 26.1 | 100.5 | 99.9 | 94.3 | 85.9 | 86.6 | 86.6 | 85.8 | 85.3 | 78.1 | 88.2 |
| FVIII (2.5%) | 147.7 | 98.6 | 104.8 | 105.8 | 104.9 | 106.1 | 111.5 | 117.1 | 119.5 | 106.5 | 104.3 |
| FVIII (5%) | 147.5 | 109.5 | 101.8 | 94.2 | 91.5 | 87.7 | 86.3 | 88.2 | 93.5 | 93.5 | 108.4 |
| FVIII (10%) | 122.7 | 108.7 | 107.1 | 108.2 | 107.6 | 106.6 | 109.3 | 110.0 | 106.1 | 110.0 | 117.9 |
| FVIII (25%) | 90.7 | 101.0 | 100.4 | 100.7 | 105.1 | 106.0 | 103.7 | 106.0 | 105.2 | 107.9 | 96.9 |
| FVIII (50%) | 70.4 | 90.9 | 93.8 | 97.3 | 100.3 | 102.7 | 102.1 | 97.4 | 98.2 | 109.7 | 104.2 |

B: pAB

| | pAB5% | pAB10% | pAB20% | pAB30% | pAB40% | pAB50% | pAB60% | pAB70% | pAB80% | pAB90% | pAB95% |
|---|---|---|---|---|---|---|---|---|---|---|---|
| FVIII (0%) | 410.8 | 124.9 | 126.1 | 106.6 | 78.8 | 76.3 | 83.1 | 82.0 | 72.4 | 37.9 | 166.1 |
| FVIII (0.25%) | 47.2 | 107.6 | 103.4 | 102.2 | 126.9 | 134.0 | 133.0 | 131.9 | 140.2 | 261.3 | 246.4 |
| FVIII (0.5%) | 203.8 | 80.0 | 81.0 | 87.6 | 85.9 | 86.0 | 84.9 | 83.5 | 76.8 | 50.7 | 43.5 |
| FVIII (0.75%) | 224.7 | 105.0 | 105.7 | 108.9 | 98.3 | 93.7 | 92.6 | 90.6 | 88.7 | 74.7 | 78.3 |
| FVIII (1%) | 27.5 | 101.9 | 105.0 | 98.5 | 86.3 | 87.0 | 86.6 | 85.6 | 85.7 | 78.1 | 88.6 |
| FVIII (2.5%) | 140.5 | 94.7 | 103.8 | 105.5 | 103.8 | 105.0 | 111.6 | 118.1 | 120.7 | 106.4 | 104.1 |
| FVIII (5%) | 158.0 | 117.8 | 107.3 | 96.4 | 92.8 | 87.2 | 85.4 | 87.4 | 93.5 | 93.3 | 108.7 |
| FVIII (10%) | 125.3 | 109.7 | 106.9 | 108.3 | 107.2 | 105.6 | 109.2 | 110.0 | 105.5 | 110.0 | 118.2 |
| FVIII (25%) | 88.6 | 99.7 | 98.3 | 98.6 | 105.2 | 106.6 | 103.6 | 106.5 | 105.1 | 107.9 | 96.6 |
| FVIII (50%) | 68.3 | 89.0 | 91.9 | 95.9 | 99.6 | 103.3 | 102.5 | 96.8 | 97.5 | 109.9 | 104.1 |

### 5.2.3) W Flattening pAWg

Table 10A shows a comparison ratio (accuracy) (%), to the actual concentration, of the FVIII concentration calculated using the W flattening pAWg based on the centroid point in each calculation target region in specimens respectively having different FVIII concentrations. In the table, cases having the accuracy within 100 ± 10% are grayed. For comparison, Table 10B similarly shows the accuracy obtained using the W flattening pAW based on the weighted average point. It was suggested that pAWg is a parameter correlated with the FVIII concentration although rather inferior to pAW.

TABLE 10

A: pAWg

| | pAWg5 % | pAWg10 % | pAWg20 % | pAWg30 % | pAWg40 % | pAWg50 % | pAWg60 % | pAWg70 % | pAWg80 % | pAWg90 % | pAWg95 % |
|---|---|---|---|---|---|---|---|---|---|---|---|
| FVIII (0%) | 387.3 | 87.7 | 84.5 | 83.4 | 86.3 | 82.3 | 76.9 | 65.5 | 39.7 | 48.2 | 180.8 |
| FVIII (0.25%) | 55.8 | 122.1 | 121.8 | 126.6 | 127.4 | 131.2 | 127.9 | 140.2 | 228.9 | 284.1 | 249.8 |
| FVIII (0.5%) | 242.9 | 87.7 | 89.2 | 87.3 | 86.1 | 83.9 | 85.0 | 72.6 | 54.5 | 43.6 | 40.3 |
| FVIII (0.75%) | 240.5 | 97.4 | 98.8 | 96.0 | 94.3 | 92.3 | 89.4 | 91.6 | 80.1 | 79.5 | 89.9 |
| FVIII (1%) | 15.4 | 87.8 | 88.3 | 85.5 | 88.2 | 87.3 | 90.5 | 90.0 | 82.8 | 78.3 | 78.0 |
| FVIII (2.5%) | 171.4 | 107.2 | 105.7 | 106.3 | 107.7 | 110.6 | 113.6 | 112.5 | 111.1 | 107.9 | 132.0 |
| FVIII (5%) | 137.2 | 92.3 | 88.7 | 92.5 | 87.5 | 88.9 | 90.1 | 96.3 | 87.5 | 95.6 | 90.1 |
| FVIII (10%) | 123.4 | 105.5 | 105.9 | 109.0 | 112.3 | 110.6 | 108.0 | 112.4 | 100.0 | 103.1 | 103.3 |
| FVIII (25%) | 93.5 | 108.6 | 105.2 | 102.8 | 106.7 | 106.4 | 105.4 | 102.0 | 111.2 | 106.5 | 116.0 |
| FVIII (50%) | 73.4 | 96.3 | 101.0 | 100.1 | 97.1 | 97.4 | 97.5 | 96.0 | 111.9 | 114.8 | 99.2 |

B: pAW

| | pAW5% | pAW10% | pAW20% | pAW30% | pAW40% | pAW50% | pAW60% | pAW70% | pAW80% | pAW90% | pAW95% |
|---|---|---|---|---|---|---|---|---|---|---|---|
| FVIII (0%) | 445.3 | 136.4 | 144.8 | 119.9 | 80.1 | 88.8 | 167.3 | 93.8 | 69.4 | 31.7 | 190.9 |
| FVIII (0.25%) | 47.8 | 99.1 | 97.2 | 96.9 | 132.5 | 139.4 | 93.8 | 89.3 | 96.6 | 226.6 | 197.6 |
| FVIII (0.5%) | 197.0 | 77.2 | 79.5 | 89.0 | 93.8 | 97.5 | 110.5 | 118.2 | 111.4 | 56.3 | 35.2 |
| FVIII (0.75%) | 229.0 | 109.6 | 112.0 | 116.4 | 102.2 | 94.7 | 104.1 | 96.5 | 99.7 | 73.4 | 94.5 |
| FVIII (1%) | 26.8 | 109.2 | 108.6 | 99.6 | 80.2 | 81.8 | 89.3 | 93.1 | 85.7 | 79.4 | 82.2 |
| FVIII (2.5%) | 134.1 | 91.2 | 103.3 | 98.8 | 90.9 | 96.9 | 120.9 | 122.9 | 119.3 | 119.6 | 155.0 |
| FVIII (5%) | 174.1 | 133.6 | 112.7 | 102.1 | 91.2 | 77.4 | 80.5 | 78.1 | 83.9 | 77.9 | 133.7 |
| FVIII (10%) | 126.9 | 110.1 | 104.4 | 104.0 | 100.4 | 98.8 | 105.3 | 109.9 | 107.2 | 134.2 | 117.2 |
| FVIII (25%) | 88.4 | 94.7 | 94.9 | 100.8 | 109.5 | 112.6 | 100.6 | 109.5 | 110.6 | 112.2 | 95.1 |
| FVIII (50%) | 66.0 | 85.9 | 92.1 | 94.5 | 107.5 | 114.0 | 100.7 | 91.4 | 91.7 | 96.0 | 80.1 |

### 5.3) Relationship Between Coagulation Factor Concentration and Parameter

As described above, a coagulation factor concentration could be measured using the parameters related to the centroid points in the linear curve and the quadratic curve. In particular, a parameter related to the centroid point calculated from the linear curve has high correlation with the coagulation factor concentration, and hence it was revealed that a coagulation factor concentration can be calculated with high accuracy by using such a parameter.

### Example 3 Evaluation of Coagulation Time Elongation Factor

### 1) Preparation of Mixed Specimen

As test specimens, a specimen having blood coagulation abnormality: 8 FVIII deficient plasma specimens (FVIII group), 4 LA positive plasma specimens (LA group), and 8 FVIII inhibitor positive plasma (Inhibitor group) were used. As the FVIII deficient plasma, Factor VIII Deficient Plasma (George King BioMedical, Inc.) was used. As the LA positive plasma, Positive Lupus Anticoagulant Plasma (George King Biomedical, Inc.) was used. As the FVIII inhibitor positive plasma, Factor VIII Deficient with Inhibitor (George King Biomedical, Inc.) having an inhibitor titer of from 4.6 to 108 (BU/mL) was used. As the normal specimen, commercially available normal plasma (CRYOcheck Pooled Normal Plasma; Precision BioLogic Incorporated) was used.

### 2) Heat Treatment

CP3000 (manufactured by Sekisui Medical Co., Ltd.) was set to perform a heat treatment for 12 minutes. In a normal setting mode, a heat treatment time after collecting 25 µL each of the test specimen and the normal specimen was 45 seconds at 37° C., but in the present mode, the heating time is increased to 12 minutes at 37° C. A mixed specimen subjected to the heat treatment for 12 minutes was used as a heated specimen, and a mixed specimen measured in the normal setting mode was used as an unheated specimen.

### 3) Coagulation Reaction Measurement and Data Analysis

Through the same procedures as those of Example 1, the coagulation reaction measurement and the data analysis of the heated specimen and the unheated specimen were performed. A parameter related to the centroid point was calculated from a linear curve and a quadratic curve. A calculation target threshold was set within a range from 5 to 90% of Vmax, Amax, or Amin (100%). The same calculation target threshold was used to calculate a parameter related to a weighted average point. Assuming that a parameter obtained from the unheated specimen was Pa and the corresponding parameter obtained from the heated specimen was Pb, a parameter ratio Pb/Pa and a parameter difference Pb - Pa were obtained.

### 4) Influence of Elongation Factor on Parameter

Between the FVIII group and the Inhibitor group, and between the LA group and the Inhibitor group, a difference in distribution of the parameter ratio (Pb/Pa) and the parameter difference (Pb - Pa) was evaluated. It was determined whether the distribution of each group was homoscedastic or heteroscedastic by F test (significance level of 1%), and subsequently, T test (two-sided) was performed to calculate a P value of each parameter ratio (Pb/Pa) and parameter difference (Pb - Pa) between the FVIII group and the Inhibitor group, and between the LA group and the Inhibitor group. Furthermore, a P value of a distribution difference of Pa and Pb between the FVIII group and the LA group was calculated.

Examples of the distributions of Pa, Pb, Pb/Pa and Pb - Pa of parameters vHg60, pHg60, mHg60, and vABg5 are illustrated in FIGS. 15-1 to FIGS. 15-4. As for all of these parameters, Pa and Pb had statistically significantly different distributions between the FVIII group and the LA group. More specifically, Pa and Pb of the FVIII group were at the same level as in a normal specimen (no data). As for all the parameters, Pb/Pa was distributed in the vicinity of 1 in the FVIII group and the LA group, but was smaller than 1 in the Inhibitor group, and was thus distributed in the Inhibitor group differently from in the FVIII group or the LA group. As for vHg60, pHg60 and vABg5, the distribution of Pb/Pa was statistically significantly different both between the FVIII group and the Inhibitor group and between the LA group and the Inhibitor group. In particular, a low P value was obtained for vHg60 and vABg5. As for mHg60, the distribution of Pb/Pa was statistically significantly different between the FVIII group and the Inhibitor group. Similarly, as for all the parameters, Pb - Pa was distributed in the vicinity of 0 in the FVIII group and the LA group, but was smaller than 0 in the Inhibitor group, and was thus distributed in the Inhibitor group significantly different from in the FVIII group or the LA group. In particular, as for vHg60, the distribution of Pb - Pa was statistically significantly different.

Accordingly, the FVIII group, the LA group and the Inhibitor group can be discriminated from one another by using Pa or Pb of the above parameters. For example, the FVIII group or the LA group can be discriminated from the Inhibitor group based on that Pb/Pa is about 1 or Pb - Pa is about 0. Subsequently, the FVIII group and the LA group can be discriminated from each other by regarding a group having Pa or Pb at the same level as in a normal specimen (not a mixed specimen) as the FVIII group.

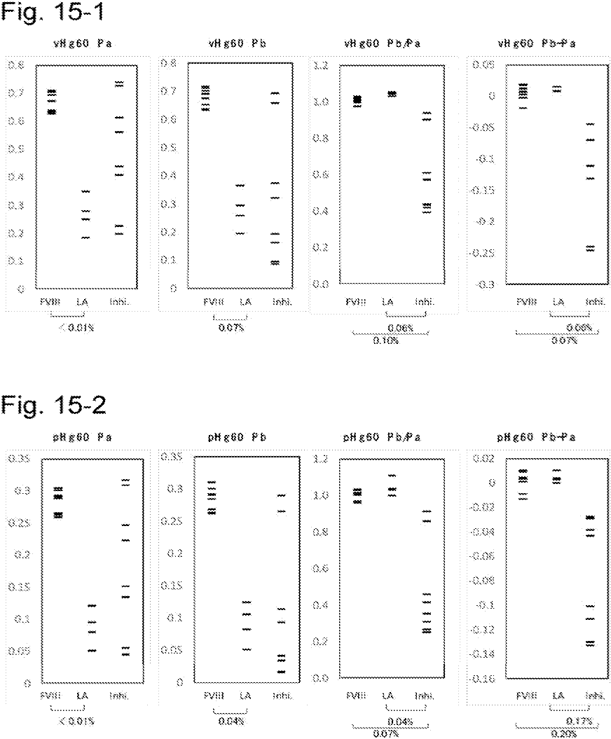

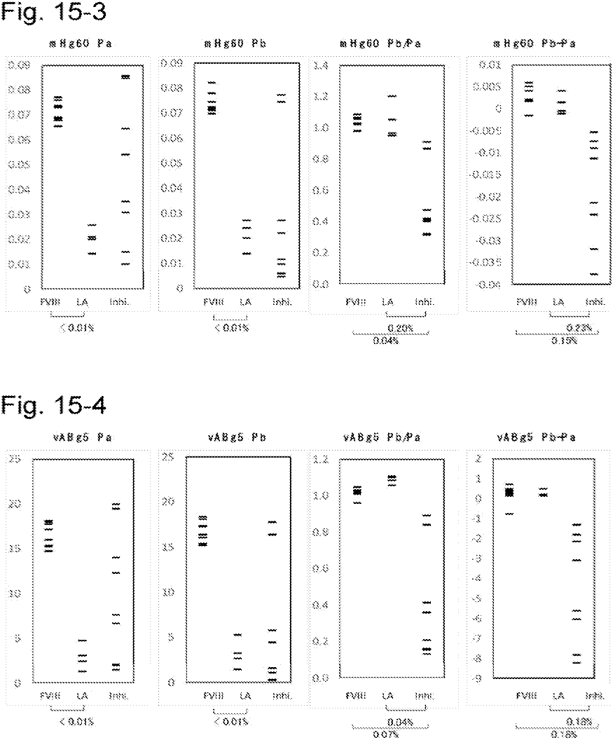

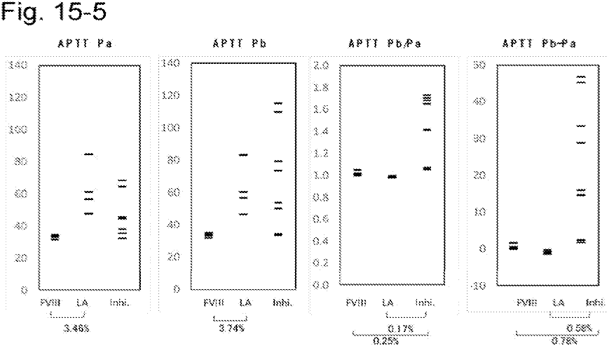

For comparison, examples of the distributions of Pa, Pb, Pb/Pa and Pb - Pa of the parameter APTT are illustrated in FIGS. 15-5. No significant difference was found in the distributions of Pa and Pb between the FVIII group and the LA group. Besides, no significant difference was found in the distributions of Pb/Pa and Pb - Pa between the FVIII group or the LA group and the Inhibitor group.

With respect to each parameter, accuracy in discrimination based on Pb/Pa among the FVIII group, the LA group, and the Inhibitor group was evaluated. A P value (i) of a distribution difference of Pb/Pa between the FVIII group and the Inhibitor group, a P value (ii) of a distribution difference of Pb/Pa between the LA group and the Inhibitor group, and a P value (iii) of a distribution difference of Pa between the FVIII group and the LA group were obtained. Based on the P values (i) to (iii), the parameter was evaluated as follows: [A] All the P values were less than 0.01%; [B] all the P values were 0.01% or more and less than 0.1%; [C] all the P values were 0.1% or more and less than 1%; and [D] the other cases. Results are shown in Tables 11 to 12. There was no large difference in tendency of the P values between the parameters related to the centroid point (Table 11) and the parameters related to the weighted average point (Table 12). In the linear curve, the parameters except for the peak width was found to have significance. In the quadratic curve, as for the positive peak, the parameters except for the peak width and the time rate showed good results, and as for the negative peak, the height alone showed good results.

TABLE 11

[Pb/Pa]

| Parameters related to Linear Curve | | | Calculation Target Threshold (%) | | | | | | | | | |
|---|---|---|---|---|---|---|---|---|---|---|---|---|
| | | | 5 | 10 | 20 | 30 | 40 | 50 | 60 | 70 | 80 | 90 |
| With Correction Processing | Peak Width | vWg | D | D | D | D | D | D | D | D | D | D |
| | Time Rate | vTWg | C | C | C | C | C | B | D | D | D | D |
| | Height | vHg | C | C | C | C | C | C | B | B | B | B |
| | B flattening | vABg | B | B | B | B | B | B | B | C | D | D |
| | W flattening | vAWg | B | B | B | B | B | B | B | D | D | D |
| Without Correction Processing | Height | RvHg | C | C | C | C | C | C | C | C | B | B |
| | B flattening | RvABg | B | B | B | B | B | B | B | C | D | D |
| | W flattening | RvAWg | B | B | B | B | B | B | B | D | D | D |

| Parameters related to Quadratic Curve (positive peak) | | | Calculation Target Threshold (%) | | | | | | | | | |
|---|---|---|---|---|---|---|---|---|---|---|---|---|
| | | | 5 | 10 | 20 | 30 | 40 | 50 | 60 | 70 | 80 | 90 |
| With Correction Processing | Peak Width | pWg | D | D | D | D | D | D | D | D | D | D |
| | Time Rate | pTWg | D | D | D | D | D | D | D | D | D | D |
| | Height | pHg | B | B | B | B | B | B | B | B | B | B |
| | B flattening | pABg | C | B | B | B | C | C | C | C | C | C |
| | W flattening | pAWg | B | B | C | C | C | C | C | C | C | C |
| Without Correction Processing | Height | RpHg | B | B | B | B | B | B | B | B | B | B |
| | B flattening | RpABg | B | B | B | B | B | B | B | C | B | C |
| | W flattening | RpAWg | B | B | B | B | C | C | B | C | C | C |

| Parameters related to Quadratic Curve (negative peak) | | | Calculation Target Threshold (%) | | | | | | | | | |
|---|---|---|---|---|---|---|---|---|---|---|---|---|
| | | | 5 | 10 | 20 | 30 | 40 | 50 | 60 | 70 | 80 | 90 |
| With Correction Processing | Peak Width | mWg | D | D | D | D | D | D | D | D | D | D |
| | Time Rate | mTWg | D | D | D | D | D | D | D | D | D | D |
| | Height | mHg | C | C | C | C | C | C | C | C | C | C |
| | B flattening | mABg | D | D | D | D | D | D | D | D | D | D |
| | W flattening | mAWg | D | D | D | D | D | D | D | D | D | D |
| Without Correction Processing | Height | RmHg | B | B | C | B | B | B | B | B | B | B |
| | B flattening | RmABg | D | D | D | D | D | D | D | D | D | D |
| | W flattening | RmAWg | D | D | D | D | D | D | D | D | D | D |

TABLE 12

[Pb/Pa]

| Parameters related to Linear Curve | | | Calculation Target Threshold (%) | | | | | | | | | |
|---|---|---|---|---|---|---|---|---|---|---|---|---|
| | | | 5 | 10 | 20 | 30 | 40 | 50 | 60 | 70 | 80 | 90 |
| With Correction Processing | Peak Width | vW | D | D | D | D | D | C | C | D | D | D |
| | Time Rate | vTW | C | C | C | C | B | D | D | D | D | D |
| | Height | vH | C | C | C | C | C | C | C | C | B | B |
| | B flattening | vAB | B | B | B | B | B | B | B | C | D | D |
| | W flattening | vAW | B | B | B | B | B | C | B | C | D | D |
| Without Correction Processing | Height | RvH | C | C | C | C | C | C | C | C | C | B |
| | B flattening | RvAB | B | B | B | B | B | B | B | C | D | D |
| | W flattening | RvAW | B | B | B | B | B | B | B | C | D | D |

| Parameters related to Quadratic Curve (positive peak) | | | Calculation Target Threshold (%) | | | | | | | | | |
|---|---|---|---|---|---|---|---|---|---|---|---|---|
| | | | 5 | 10 | 20 | 30 | 40 | 50 | 60 | 70 | 80 | 90 |
| With Correction Processing | Peak Width | pW | D | D | D | D | D | C | C | D | D | D |
| | Time Rate | pTW | D | D | D | D | D | D | D | D | D | D |
| | Height | pH | C | B | B | B | B | B | B | B | B | B |
| | B flattening | pAB | C | B | B | B | C | C | C | C | C | C |
| | W flattening | pAW | B | B | B | C | B | B | C | C | C | C |
| Without Correction Processing | Height | RpH | B | B | B | B | B | B | B | B | B | B |
| | B flattening | RpAB | B | B | B | B | B | B | B | C | B | C |
| | W flattening | RpAW | B | B | B | B | B | B | B | C | A | B |

| Parameters related to Quadratic Curve (negative peak) | | | Calculation Target Threshold (%) | | | | | | | | | |
|---|---|---|---|---|---|---|---|---|---|---|---|---|
| | | | 5 | 10 | 20 | 30 | 40 | 50 | 60 | 70 | 80 | 90 |
| With Correction Processing | Peak Width | mW | D | D | D | D | D | D | D | D | D | D |
| | Time Rate | mTW | D | D | D | D | D | D | D | D | D | D |
| | Height | mH | B | B | C | C | C | C | C | C | C | C |
| | B flattening | mAB | D | D | D | D | D | D | D | D | D | D |
| | W flattening | mAW | D | D | D | D | D | D | D | D | D | D |
| Without Correction Processing | Height | RmH | B | B | C | C | B | C | B | B | B | B |
| | B flattening | RmAB | D | D | D | D | D | D | D | D | D | D |
| | W flattening | RmAW | D | D | D | D | D | D | D | D | D | D |

Similarly, as for each parameter, accuracy in discrimination based on Pb - Pa among the FVIII group, the LA group, and the Inhibitor group was evaluated. Results are shown in Tables 13 to 14. There was no large difference in tendency of the P values between the parameters related to the centroid point (Table 13) and the parameters related to the weighted average point (Table 14).

TABLE 13

[Pb-Pa]

| Parameters related to Linear Curve | | | Calculation Target Threshold (%) | | | | | | | | | |
|---|---|---|---|---|---|---|---|---|---|---|---|---|
| | | | 5 | 10 | 20 | 30 | 40 | 50 | 60 | 70 | 80 | 90 |
| With Correction Processing | Peak Width | vWg | D | D | D | D | D | D | D | D | D | D |
| | Time Rate | vTWg | C | C | B | B | B | B | D | D | D | D |
| | Height | vHg | B | B | B | B | B | B | B | B | B | B |
| | B flattening | vABg | C | C | C | C | C | C | C | C | D | D |
| | W flattening | vAWg | C | C | C | C | C | C | C | C | D | D |
| Without Correction Processing | Height | RvHg | B | B | B | B | B | B | B | B | B | B |
| | B flattening | RvABg | B | B | B | B | B | C | B | C | D | D |
| | W flattening | RvAWg | C | B | B | B | B | B | C | C | D | D |

| Parameters related to Quadratic Curve (positive peak) | | | Calculation Target Threshold (%) | | | | | | | | | |
|---|---|---|---|---|---|---|---|---|---|---|---|---|
| | | | 5 | 10 | 20 | 30 | 40 | 50 | 60 | 70 | 80 | 90 |
| With Correction Processing | Peak Width | pWg | D | D | D | D | D | D | D | D | D | D |
| | Time Rate | pTWg | D | D | D | D | D | D | D | D | D | D |
| | Height | pHg | C | C | C | C | C | C | C | C | C | C |
| | B flattening | pABg | C | C | C | C | C | C | C | C | D | D |
| | W flattening | pAWg | C | C | C | C | C | C | C | C | C | D |
| Without Correction Processing | Height | RpHg | B | B | B | B | B | B | B | B | B | B |
| | B flattening | RpABg | C | C | C | C | C | C | C | C | C | C |
| | W flattening | RpAWg | C | C | C | C | C | C | C | C | C | C |

| Parameters related to Quadratic Curve (negative peak) | | | Calculation Target Threshold (%) | | | | | | | | | |
|---|---|---|---|---|---|---|---|---|---|---|---|---|
| | | | 5 | 10 | 20 | 30 | 40 | 50 | 60 | 70 | 80 | 90 |
| With Correction Processing | Peak Width | mWg | D | D | D | D | D | D | D | D | D | D |
| | Time Rate | mTWg | D | D | D | D | D | D | D | D | D | D |
| | Height | mHg | C | C | C | C | C | C | C | C | C | C |
| | B flattening | mABg | D | D | D | D | D | D | D | D | D | D |
| | W flattening | mAWg | D | D | D | D | D | D | D | D | D | D |
| Without Correction Processing | Height | RmHg | C | C | C | C | C | C | C | C | C | C |
| | B flattening | RmABg | D | D | D | D | D | D | D | D | D | D |
| | W flattening | RmAWg | D | D | D | D | D | D | D | D | D | D |

TABLE 14

[Pb-Pa]

| Parameters related to Linear Curve | | | Calculation Target Threshold (%) | | | | | | | | | |
|---|---|---|---|---|---|---|---|---|---|---|---|---|
| | | | 5 | 10 | 20 | 30 | 40 | 50 | 60 | 70 | 80 | 90 |
| With Correction Processing | Peak Width | vW | D | D | D | D | D | C | C | D | D | D |
| | Time Rate | vTW | C | B | B | B | B | D | D | D | D | D |
| | Height | vH | B | B | B | B | B | B | B | B | B | B |
| | B flattening | vAB | C | C | C | C | C | C | C | C | D | D |
| | W flattening | vAW | C | C | C | C | C | C | C | C | D | D |
| Without Correction Processing | Height | RvH | B | B | B | B | B | B | B | B | B | A |
| | B flattening | RvAB | B | C | B | C | C | C | B | C | D | D |
| | W flattening | RvAW | B | C | B | C | C | C | B | C | D | D |

| Parameters related to Quadratic Curve (positive peak) | | | Calculation Target Threshold (%) | | | | | | | | | |
|---|---|---|---|---|---|---|---|---|---|---|---|---|
| | | | 5 | 10 | 20 | 30 | 40 | 50 | 60 | 70 | 80 | 90 |
| With Correction Processing | Peak Width | pW | D | D | D | D | D | D | D | D | D | D |
| | Time Rate | pTW | D | D | D | D | D | D | D | D | D | D |
| | Height | pH | C | C | C | C | C | C | C | C | C | C |
| | B flattening | pAB | C | C | C | C | C | C | C | C | D | D |
| | W flattening | pAW | C | C | C | C | C | C | C | C | C | C |
| Without Correction Processing | Height | RpH | B | B | B | B | B | B | B | B | B | B |
| | B flattening | RpAB | C | B | C | C | C | C | C | C | C | C |
| | W flattening | RpAW | C | B | C | C | C | C | B | C | B | C |

| Parameters related to Quadratic Curve (negative peak) | | | Calculation Target Threshold (%) | | | | | | | | | |
|---|---|---|---|---|---|---|---|---|---|---|---|---|
| | | | 5 | 10 | 20 | 30 | 40 | 50 | 60 | 70 | 80 | 90 |
| With Correction Processing | Peak Width | mW | D | D | D | D | D | D | D | D | D | D |
| | Time Rate | mTW | D | D | D | D | D | D | D | D | D | D |
| | Height | mH | B | C | C | C | C | C | C | C | C | C |
| | B flattening | mAB | D | D | D | D | D | D | D | D | D | D |
| | W flattening | mAW | D | D | D | D | D | D | D | D | D | D |
| Without Correction Processing | Height | RmH | A | C | C | C | C | C | C | B | C | C |
| | B flattening | RmAB | D | D | D | D | D | D | D | D | D | D |
| | W flattening | RmAW | D | D | D | D | D | D | D | D | D | D |

Based on these results, it was revealed that a coagulation factor deficient specimen, an LA positive specimen, and a coagulation factor inhibitor specimen can be discriminated from one another by using parameters related to a centroid point obtained from a heated specimen and an unheated specimen, in particular, parameters related to the centroid point obtained from a linear curve.

### Example 4 Measurement of Coagulation Factor Inhibitor Titer

### 1) Test Specimen

As a test specimen, a specimen obtained by diluting FVIII inhibitor plasma (Factor VIII Deficient with Inhibitor, George King Biomedical, Inc.) with FVIII deficient plasma (Factor VIII Deficient, George King Biomedical, Inc.) was used.

### 2) Calculation of Inhibitor Titer (Bethesda Unit)

The inhibitor titer of the test specimen was calculated based on a display value (titer) of the FVIII inhibitor plasma, and a dilution ratio of the FVIII inhibitor plasma in the test specimen, assuming that the titer of the FVIII deficient plasma was zero. The thus obtained value was defined as the measured titer of the test specimen. The inhibitor titer was classified into “low,” “intermediate,” and “high” in accordance with a value in the Bethesda unit (BU/mL) as follows:
low: from 0.3 to 1.6 (BU/mL)
intermediate: from 2.0 to 40.5 (BU/mL)
high: from 66 to 302 (BU/mL)

### 3) Preparation and Heat Treatment of Mixed Specimen

As a normal specimen, normal pooled plasma in which an FVIII concentration and an FXI concentration could be regarded as 100% was used. Each test specimen was mixed with the normal specimen in a volume ratio of 1:1 to prepare a mixed specimen. A part of the mixed specimen was taken out to be subjected to a heat treatment at 37° C. for 10 minutes, and the resultant was used as a heated specimen. A specimen not subjected to this heat treatment was used as an unheated specimen.

### 4) Coagulation Reaction Measurement and Data Analysis

Through similar procedures to those employed in Example 1, coagulation reaction measurement and data analysis of the heated specimen and the unheated specimen were performed. Parameters related to centroid points were calculated from the resultant linear curve and quadratic curve. A calculation target threshold was set in a range of from 0.5 to 90% of Vmax, Amax or Amin (100%). The same calculation target threshold was used to calculate parameters related to a weighted average point. Assuming that a parameter acquired from the unheated specimen was Pa and a corresponding parameter acquired from the heated specimen was Pb, a parameter ratio Pb/Pa was obtained.

### 5) Relationship Between Inhibitor Titer and Parameter Ratio

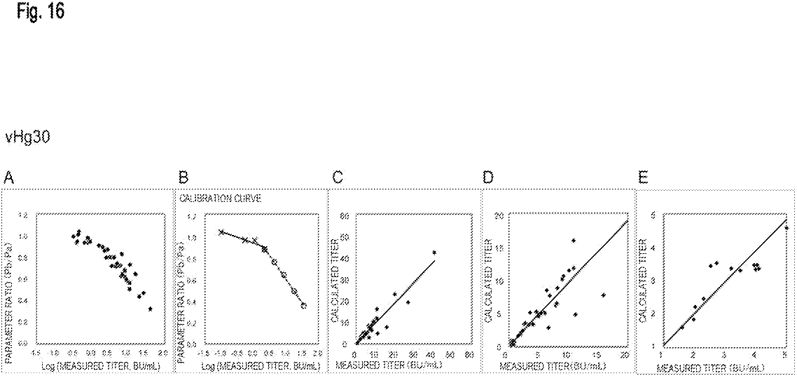

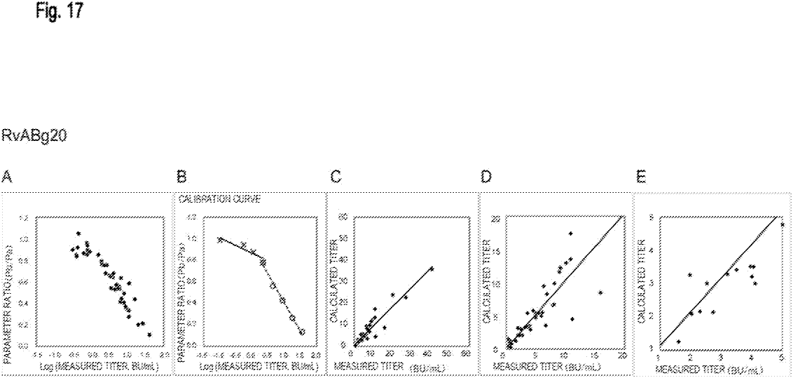

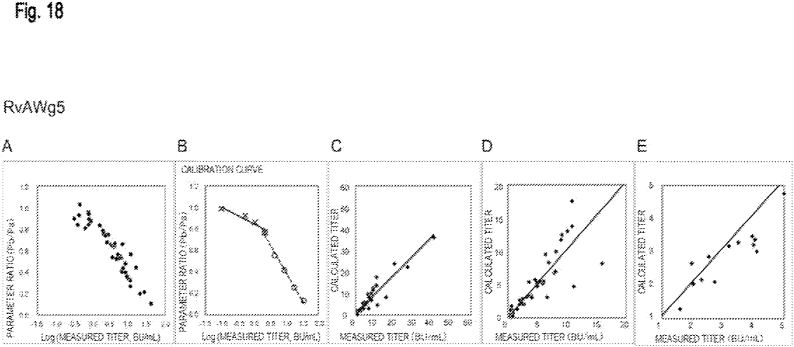

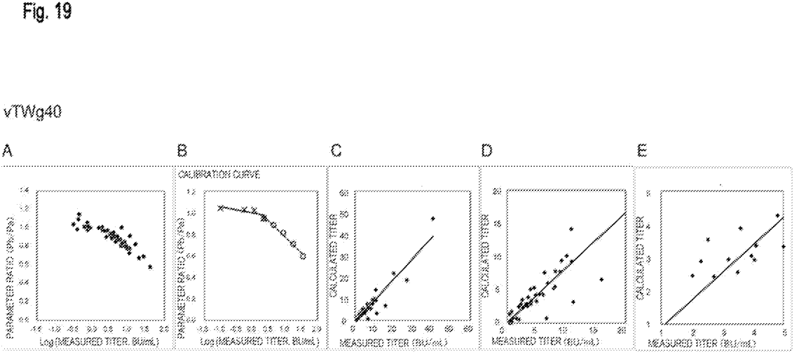

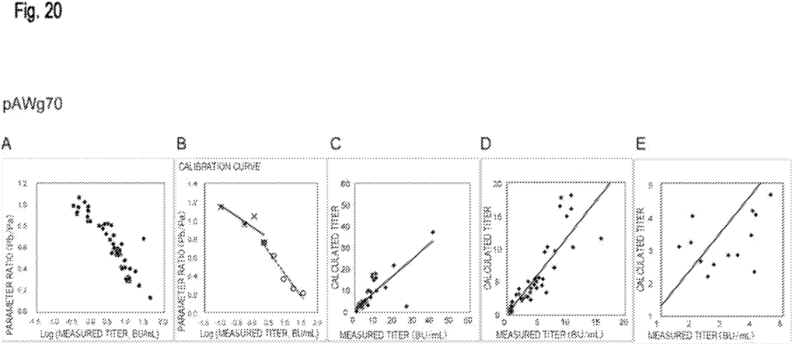

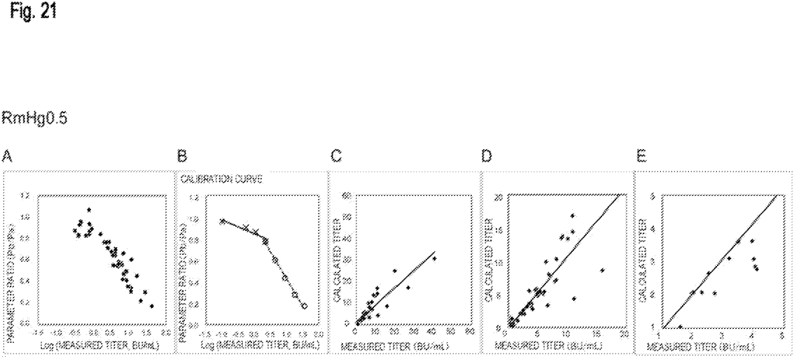

FIGS. 16A to 21A illustrate plots each of Pb/Pa of the parameter related to the centroid point acquired from each mixed specimen against a logarithm of the inhibitor titer of the test specimen contained in the mixed specimen (Log (measured titer), BU/mL). The parameter related to the centroid point used in the calculation of Pb/Pa was vHg30% in FIG. 16A, RvABg20% in FIG. 17A, RvAWg5% in FIG. 18A, vTWg40% in FIG. 19A, pAWg70% in FIG. 20A, and RmHg0.5% in FIG. 21A. As illustrated in FIGS. 16A to 21A, it was revealed that the parameter ratios Pb/Pa of the parameters related to the centroid point increase or decrease in accordance with the inhibitor titer, and that Pb/Pa has correlation with the inhibitor titer. On the other hand, the distribution of Pb/Pa had different tendency between a low inhibitor titer region and a high inhibitor titer region. This suggested that the correlation between the titer and Pb/Pa is improved by regressing the low inhibitor titer region and the high inhibitor titer region with different lines.

### 6) Creation of Calibration Curve

In the same manner as the test specimen, a specimen for calibration curve was prepared by diluting FVIII inhibitor plasma having a known FVIII inhibitor titer (Factor VIII Deficient with Inhibitor, George King Biomedical, Inc.) with FVIII deficient plasma (Factor VIII Deficient, George King Biomedical, Inc.). As the specimen for calibration curve (Cal), 8 specimens in total, that is, 1 FVIII deficient specimen, and 7 specimens respectively having FVIII inhibitor titers of 0.5, 1.1, 2.2, 4.4, 8.7, 17.4, and 34.9 (BU/mL) were used. Assuming that the inhibitor titer of the FVIII deficient specimen was 0.1, a linear regression line between a logarithm of the inhibitor titer (Log (measured titer), BU/mL) and Pb/Pa of each specimen was obtained. At this point, the specimens were divided into a low inhibitor titer group and a high inhibitor titer group, and a regression line was obtained with respect to each of the groups. A titer corresponding to the boundary between the low inhibitor titer group and the high inhibitor titer group was set to 2.2 (BU/mL). Examples of the thus created calibration curves are illustrated in FIGS. 16B to 21B. The parameters used were the same as those used in the corresponding ones of FIGS. 16A to 21A. All the calibration curves were illustrated as polygonal curves each including 2 lines.

### 7) Calculation of Inhibitor Titer Using Calibration Curve

Based on each of the thus created calibration curves, the FVIII inhibitor titer of the test specimen was calculated from Pb/Pa. FIGS. 16C to 21C illustrate plots each of a value calculated based on the calibration curve against the inhibitor titer (measured titer) of each test specimen. FIGS. 16D to 21D illustrate replots obtained using only data of titers of 20 BU/mL or less of the corresponding ones of FIGS. 16C to 21C. FIGS. 16E to 21E illustrate replots obtained using only data of titers of 5 BU/mL or less of the corresponding ones of FIGS. 16C to 21C.

The slope, the section, and a correlation coefficient of a linear regression equation, against the measured titer (x), of the calculated titer (y) based on the calibration curve created from Pb/Pa of each the parameter related to the centroid point were obtained, and the correlation of the regression equation was evaluated as shown in Table 15. As a reference, the correlation of a linear regression equation, against a measured titer (x), of a calculated titer (y) based on a calibration curve created from Pb/Pa of the parameter related to the weighted average point is shown in Table 16.

TABLE 15

Parameters having, in regression expression based on Pb/Pa, slope: within 1±0.1, section: within ±3, correlation coefficient 0.9 or more

| Curve | Parameter | Calculation Target Threshold (%) | | | | | | | | | | | |
|---|---|---|---|---|---|---|---|---|---|---|---|---|---|
| | | 0.5 | 1 | 5 | 10 | 20 | 30 | 40 | 50 | 60 | 70 | 80 | 90 |
| Linear Curve | vHg | A | A | A | A | A | A | A | A | A | A | A | A |
| | vWg | | | | | | | | | | | | |
| | vABg | | A | C | C | C | | | | | | | |
| | vAWg | C | C | C | | | | | | | | | |
| | vTWG | | A | A | | A | A | A | A | A | | | |
| Quadratic Curve (positive peak | pHg | | | | | | | | | | | | |
| | pWg | | | | | | | | | | | | |
| | pABg | | | | | | | | | | | | |
| | pAWg | | | | | | | | | | | | |
| | pTWg | | | | | | | | | | | | |
| Quadratic Curve (negative peak | mHg | | | | | | | | | | | | |
| | mWg | | | | | | | | | | | | |
| | mABg | | | | | | | | | | | | |
| | mAWg | | | | | | | | | | | | |

Parameters having, in regression expression based on Pb/Pa, Slope: within 1±0.2, section: within ±3, correlation coefficient: 0.8 or more

| Curve | Parameter | Calculation Target Threshold (%) | | | | | | | | | | | |
|---|---|---|---|---|---|---|---|---|---|---|---|---|---|
| | | 0.5 | 1 | 5 | 10 | 20 | 30 | 40 | 50 | 60 | 70 | 80 | 90 |
| Linear Curve | vHg | A | A | A | A | A | A | A | A | A | A | A | A |
| | vWg | | | | | | | | A | A | A | | |
| | vABg | | A | A | A | A | A | A | A | A | A | | |
| | vAWg | A | A | A | A | A | A | A | A | A | A | C | |
| | vTWg | A | A | A | A | A | A | A | A | A | | | |
| Quadratic Curve (positive peak | pHg | | | | | | | | | | | | |
| | pWg | | | | | | | | | | | | |
| | pABg | | | | | | | | | | | | |
| | pAWg | | | | | | | | | | C | | |
| | pTWg | | | | | | | | | | | | |
| Quadratic Curve (negative peak | mHg | C | | | | | | | | | | | |
| | mWg | | | | | | | | | | | | |
| | mAGg | A | | | | | | | | | | | |
| | mAWg | | | | | | | | | | | | |

A: both of with and without correction processing
B: with correction processing
C: without correction processing

TABLE 16

Parameters having, in regression expression based on Pb/Pa, Slope: within 1±0.1, section: within ±3, correlation coefficient: 0.9 or more

| Curve | Parameter | Calculation Target Threshold (%) | | | | | | | | | | | |
|---|---|---|---|---|---|---|---|---|---|---|---|---|---|
| | | 0. 5 | 1 | 5 | 10 | 20 | 30 | 40 | 50 | 60 | 70 | 80 | 90 |
| Linear Curve | vH | | | B | B | B | A | A | A | A | A | A | C |
| | vW | | | | | | | | | | | | |
| | vAB | | | A | A | C | | C | | | | | |
| | vAW | | | C | A | C | | | C | A | A | | |
| | vTW | | | | | A | A | A | | | | | |
| Quadratic Curve (positive peak) | pH | | | | | | | | | | | | |
| | pW | | | | | | | | | | | | |
| | pAB | | | | | | | | | | | | |
| | pAW | | | | | | | | | | | | |
| | pTW | | | | | | | | | | | | |
| Quadratic Curve (negative peak) | mH | | | | | | | | | | | | |
| | mW | | | | | | | | | | | | |
| | mAB | | | | | | | | | | | | |
| | mAW | | | | | | | | | | | | |

Parameters having, in regression expression based on Pb/Pa, slope: within 1±0.2, section: within ±3, correlation coefficient: 0.8 or more

| Curve | Parameter | Calculation Target Threshold (%) | | | | | | | | | | | |
|---|---|---|---|---|---|---|---|---|---|---|---|---|---|
| | | 0. 5 | 1 | 5 | 10 | 20 | 30 | 40 | 50 | 60 | 70 | 80 | 90 |
| Linear Curve | vH | | | A | A | A | A | A | A | A | A | A | A |
| | vW | | | | | | | | | | | | |
| | vAB | | | A | A | A | A | A | A | A | A | | |
| | vAW | | | A | A | A | A | A | A | A | A | A | |
| | vTW vTW | | | A | A | A | A | A | | | | | |
| Quadratic Curve (positive peak) | pH | | | | | | | | | | | | |
| | pW | | | | | | | | | | | | |
| | pAB | | | | | | | | | | | | |
| | pAW | | | | | | | | | | A | | |
| | pTW | | | | | | | | | | | | |
| Quadratic Curve (negative peak) | mH | | | | C | A | | | | | | | |
| | mW | | | | | | | | | | | | |
| | mAB | | | | | | | | | | | | |
| | mAW | | | | | | | | | | | | |

A: both of with and without correction processing
B: with correction processing
C:without correction processing

It was revealed, based on these results, that an inhibitor titer of a specimen can be measured using a parameter related to a centroid point, in particular, a parameter related to a centroid point obtained from a linear curve.

### Example 5 Evaluation of Coagulation Properties using Template Specimen

### 1) Preparation of Specimens

Analysis was performed on 34 specimens (plasmas). The 34 specimens include 24 FVIII deficient specimens (13 specimens of serious deficiency (FVIII < 1%), 8 specimens of moderate deficiency (FVIII = 1 to 5%), and 3 specimens of mild deficiency (FVIII = 5 to 40%)), and 10 specimens (Other) except for the VIII deficient specimens.

### 2) Coagulation Reaction Measurement and Data Analysis

The coagulation reaction measurement and data analysis were performed through the same procedures as in Example 1. Parameters related to a centroid point were calculated from a linear curve and a quadratic curve, and as the other parameters, the maximum value Vmax and a corresponding time VmaxT of the linear curve, and the maximum value Amax and a corresponding time AmaxT of the quadratic curve were calculated. A calculation target threshold was set in a range from 5 to 90% of the Vmax or the Amax (100%).

### 3) Template Specimen

The constitution of a template specimen used in the analysis is shown in Table 17. 43 specimens having different FVIII activity levels, and 88 specimens having normal FVIII activity but having blood coagulation time elongated due to other factors were prepared. The FVIII activity of each of the former 43 specimens corresponds to any one of serious (FVIII < 1%), moderate (FVIII = 1 to 5%), and mild (FVIII = 5 to 40%) hemophilia A, and the other (FVIII > 40% corresponding to “Other”). The latter 88 specimens are not abnormal in FVIII activity, and hence correspond to “Other” in the classification of Table 1. These 131 specimens in total were used in the analysis as template specimens to calculate parameters from theses specimens in accordance with the procedures described in the section 2).

TABLE 17

Constitution of Template Specimens

| | Number of Specimens | FVIII <1% | FVIII 1-5% | FVIII 5-40% | Other |
|---|---|---|---|---|---|
| Specimens having different FVIII Activity | 43 | 11 | 11 | 15 | 6 |
| Specimens having other factors | 88 | - | - | - | 88 |
| total | 131 | 11 | 11 | 15 | 94 |

Specimens having other factors: deficiency of coagulation factor except FVIII, heparinized plasma, and LA positive plasma

### 4) Creation of Parameter Sets

The parameters for the test specimens and the template specimens were combined to create test parameter sets and template parameter sets as follows:
(Parameter set A-1) A parameter set (of 50 parameters in total) of parameter groups (each including 10 parameters) of vTg, vHg, vB, vABg, and vTBg with the calculation target thresholds of 5%, 10%, 20%, 30%, 40%, 50%, 60%, 70%, 80%, and 90%;
(Parameter set A-2) a parameter set (of 30 parameters in total) of parameter groups (each including 10 parameters) of vB, vABg, and vTBg with the calculation target thresholds of 5%, 10%, 20%, 30%, 40%, 50%, 60%, 70%, 80%, and 90%;
(Parameter set A-3) a parameter set (of 20 parameters in total) of parameter groups (each including 10 parameters) of vB and vABg with the calculation target thresholds of 5%, 10%, 20%, 30%, 40%, 50%, 60%, 70%, 80%, and 90%;
(Parameter set A-4) a parameter set of 54 parameters obtained by adding Vmax, Amax, VmaxT, and AmaxT to the parameter set A-1;
(Parameter set B-1) a parameter set (of 25 parameters in total) of parameter groups (each including 5 parameters) of vTg, vHg, vB, vABg, and vTBg with the calculation target thresholds of 5%, 20%, 40%, 60%, and 80% ;
(Parameter set B-2) a parameter set (of 15 parameters in total) of parameter groups (each including 5 parameters) of vB, vABg, and vTBg with the calculation target thresholds of 5%, 20%, 40%, 60%, and 80%;
(Parameter set B-3 (of 10 parameters in total) of parameter groups (each including 5 parameters) of vB and vABg with the calculation target thresholds 5%, 20%, 40%, 60%, and 80%;
(Parameter set B-4) a parameter set of 29 parameters obtained by adding Vmax, Amax, VmaxT, and AmaxT to the parameter set B-1; and
(Comparative parameter set 1) a parameter set of 4 parameters of Vmax, Amax, VmaxT, and AmaxT.

The constitutions of the created parameter sets are shown in Table 18.

TABLE 18

Constitution of Parameter Set Used in Analysis

| Parameter Set A ○ | | | | Parameter Set B ● | | | |
|---|---|---|---|---|---|---|---|
| | | A-1/B-1 (vHq, vTq, vB, vABg, vTBg) | A-2/B-2 (vB, vABg, vTBg) | | A-3/B-3 (vB, vABg) | A-4/B-4 (all) | Comparison (conventional) |
| Conventional Parameter | VmaxT | | | | | ○● | ○ |
| | Vmax | | | | | ○● | ○ |
| | AmaxT | | | | | ○● | ○ |
| | Amax | | | | | ○● | ○ |
| Centroid Time vTg | 5% | ○● | | | | ○● | |
| | 10% | ○ | | | | ○ | |
| | 20% | ○● | | | | ○● | |
| | 30% | ○ | | | | ○ | |
| | 40% | ○● | | | | ○● | |
| | 50% | ○ | | | | ○ | |
| | 60% | ○● | | | | ○● | |
| | 70% | ○ | | | | ○ | |
| | 80% | ○● | | | | ○● | |
| | 90% | ○ | | | | ○ | |
| Centroid Height vHg | 5% | ○● | | | | ○● | |
| | 10% | ○ | | | | ○ | |
| | 20% | ○● | | | | ○● | |
| | 30% | ○ | | | | ○ | |
| | 40% | ○● | | | | ○● | |
| | 50% | ○ | | | | ○ | |
| | 60% | ○● | | | | ○● | |
| | 70% | ○ | | | | ○ | |
| | 80% | ○● | | | | ○● | |
| | 90% | ○ | | | | ○ | |
| Peak Width vB | 5% | ○● | ○● | | ○● | ○● | |
| | 10% | ○ | ○ | | ○ | ○ | |
| | 20% | ○● | ○● | | ○● | ○● | |
| | 30% | ○ | ○ | | ○ | ○ | |
| | 40% | ○● | ○● | | ○● | ○● | |
| | 50% | ○ | ○ | | ○ | ○ | |
| | 60% | ○● | ○● | | ○● | ○● | |
| | 70% | ○ | ○ | | ○ | ○ | |
| | 80% | ○● | ○● | | ○● | ○● | |
| | 90% | ○ | ○ | | ○ | ○ | |
| Flattening vABg | 5% | ○● | ○● | | ○● | ○● | |
| | 10% | ○ | ○ | | ○ | ○ | |
| | 20% | ○● | ○● | | ○● | ○● | |
| | 30% | ○ | ○ | | ○ | ○ | |
| | 40% | ○● | ○● | | ○● | ○● | |
| | 50% | ○ | ○ | | ○ | ○ | |
| | 60% | ○● | ○● | | ○● | ○● | |
| | 70% | ○ | ○ | | ○ | ○ | |
| | 80% | ○● | ○● | | ○● | ○● | |
| | 90% | ○ | ○ | | ○ | ○ | |
| Time Rate vTBg | 5% | ○● | ○● | | | ○● | |
| | 10% | ○ | ○ | | | ○ | |
| | 20% | ○● | ○● | | | ○● | |
| | 30% | ○ | ○ | | | ○ | |
| | 40% | ○● | ○● | | | ○● | |
| | 50% | ○ | ○ | | | ○ | |
| | 60% | ○● | ○● | | | ○● | |
| | 70% | ○ | ○ | | | ○ | |
| | 80% | ○● | ○● | | | ○● | |
| | 90% | ○ | ○ | | | ○ | |

### 5) Determination of FVIII Activity or Abnormality of Test Specimen

With respect to each of the parameter sets A-1 to A-4, and B-1 to B-4, and the comparative parameter set 1 thus obtained, the regression analysis between the 34 test specimens and each of the template specimens was conducted. Between a test specimen and each of all the template specimens, linear regression equations of the parameter sets were obtained, and template specimens having the slope of the regression line in a range from 0.87 to 1.13 were selected therefrom. Next, from the thus selected template specimens, one having the largest correlation coefficient was selected as a template specimen having the highest correlation. The FVIII activity of the selected template specimen was determined as the FVIII activity of the test specimen. Based on the determination result, the FVIII activity level of the test specimen was classified into four stages (FVIII activity of less than 1%, from 1 to 5%, from 5 to 40%, and “Other”). Based on the FVIII activity level of the test specimen thus classified, and the actual FVIII activity level of the test specimen obtained by a one stage coagulation method, an FVIII activity level match rate and an FVIII deficiency match rate in this determination were calculated in accordance with the following expressions. The FVIII activity level match rate indicates a rate of the determined FVIII activity level of the test specimen matching the actual FVIII activity level of the test specimen, and the FVIII deficiency match rate indicates a rate of the determined presence of the FVIII deficiency of the test specimen matching the actual presence of the FVIII deficiency of the test specimen.

$$\begin{matrix}\text{FVIII activity level match rate} \left(\%\right) = \left(\text{A11} + \text{A22} +\right) \\ \left(\text{A33} + \text{A44}\right) / \text{D} \times 100\end{matrix}$$

$$\begin{matrix}\text{FVIII deficiency match rate} \left(\%\right) = \left(\text{A11} + \text{A12} + \text{A13} +\right) \\ \left(\text{A21} + \text{A22} + \text{A23} + \text{A31} + \text{A32} + \text{A33} + \text{A44}\right) / \text{D} \times 100\end{matrix}$$

| | | FVIII Activity Level (determined) | | | | |
|---|---|---|---|---|---|---|
| | | <1% | 1-5% | 5-40% | Other | Total |
| FVIII Activity Level (measured) | <1% | A11 | A12 | A13 | A14 | B1 |
| | 1-5% | A21 | A22 | A23 | A24 | B2 |
| | 5-40% | A31 | A32 | A33 | A34 | B3 |
| | Other | A41 | A42 | A43 | A44 | B4 |
| | Total | C1 | C2 | C3 | C4 | D |

Tables 19 to 21 are comparison tables between the determined FVIII activity of the test specimens and the actual FVIII activity of the test specimens. Table 19 is a comparison table using the parameter sets A-1 to A-4, Table 20 is a comparison table using the parameter sets B-1 to B-4, and Table 21 is a comparison table using the comparative parameter set 1.

TABLE 19-A1

Analysis Results with Parameter Set A-1

| | | FVIII Activity Level (determined) | | | | FVIII Activity Level Match Rate (%) | | | |
|---|---|---|---|---|---|---|---|---|---|
| | | <1% | 1-5% | 5-40% | Other | match | nonmatch | total | match rate |
| FVIII Activity Level (measured) | <1% | 12 | 1 | | | 12 | 1 | 13 | 92.3 |
| | 1-5% | | 4 | 3 | 1 | 4 | 4 | 8 | 50.0 |
| | 5-40% | | 1 | 2 | | 2 | 1 | 3 | 66.7 |
| | Other | | | | 10 | 10 | 0 | 10 | 100.0 |
| | total | 12 | 6 | 5 | 11 | 28 | 6 | 34 | 82.4 |

FVIII Deficiency match rate = 97.1% (33 specimens out of 34 specimens)

TABLE 19-A2

Analysis Results with Parameter Set A-2

| | | FVIII Activity Level (determined) | | | | FVIII Activity Level Match Rate (%) | | | |
|---|---|---|---|---|---|---|---|---|---|
| | | <1% | 1-5% | 5-40% | Other | match | nonmatch | total | match rate |
| FVIII Activity Level (measured) | <1% | 12 | | | 1 | 12 | 1 | 13 | 92.3 |
| | 1-5% | | 6 | 1 | 1 | 6 | 2 | 8 | 75.0 |
| | 5-40% | | | 2 | 1 | 2 | 1 | 3 | 66.7 |
| | Other | | | 1 | 9 | 9 | 1 | 10 | 90.0 |
| | total | 12 | 6 | 4 | 12 | 29 | 5 | 34 | 85.3 |

FVIII Deficiency match rate = 88.2% (30 specimens out of 34 specimens)

TABLE 19-A3

Analysis Results with Parameter Set A-3

| | | FVIII Activity Level (determined) | | | | FVIII Activity Level Match Rate (%) | | | |
|---|---|---|---|---|---|---|---|---|---|
| | | <1% | 1-5% | 5-40% | Other | match | nonmatch | total | match rate |
| FVIII Activity Level (measured) | <1% | 12 | | | 1 | 12 | 1 | 13 | 92.3 |
| | 1-5% | | 7 | 1 | | 7 | 1 | 8 | 87.5 |
| | 5-40% | | | 2 | 1 | 2 | 1 | 3 | 66.7 |
| | Other | | | 1 | 9 | 9 | 1 | 10 | 90.0 |
| | total | 12 | 7 | 4 | 11 | 30 | 4 | 34 | 88.2 |

FVIII Deficiency match rate = 91.2% (31 specimens out of 34 specimens)

TABLE 19-A4

Analysis Results with Parameter Set A-4

| | | FVIII Activity Level (determined) | | | | FVIII Activity Level Match Rate (%) | | | |
|---|---|---|---|---|---|---|---|---|---|
| | | <1% | 1-5% | 5-40% | Other | match | nonmatch | total | match rate |
| FVIII Activity Level (measured) | <1% | 12 | 1 | | | 12 | 1 | 13 | 92.3 |
| | 1-5% | | 4 | 4 | | 4 | 4 | 8 | 50.0 |
| | 5-40% | | 1 | 2 | | 2 | 1 | 3 | 66.7 |
| | Other | | | | 10 | 10 | 0 | 10 | 100.0 |
| | total | 12 | 6 | 6 | 10 | 28 | 6 | 34 | 82.4 |

FVIII Deficiency match rate = 100% (34 specimens out of 34 specimens)

TABLE 20-B1

Analysis Results with Parameter Set B-1

| | | FVIII Activity Level (determined) | | | | FVIII Activity Level Match Rate (%) | | | |
|---|---|---|---|---|---|---|---|---|---|
| | | <1% | 1-5% | 5-40% | Other | match | nonmatch | total | match rate |
| FVIII Activity Level (measured) | <1% | 12 | 1 | | | 12 | 1 | 13 | 92.3 |
| | 1-5% | | 5 | 1 | 2 | 5 | 3 | 8 | 62.5 |
| | 5-40% | | 1 | 2 | | 2 | 1 | 3 | 66.7 |
| | Other | | | 1 | 9 | 9 | 1 | 10 | 90.0 |
| | total | 12 | 7 | 4 | 11 | 28 | 6 | 34 | 82.4 |

FVIII Deficiency match rate = 91.2% (31 specimens out of 34 specimens)

TABLE 20-B2

Analysis Results with Parameter Set B-2

| | | FVIII Activity Level (determined) | | | | FVIII Activity Level Match Rate (%) | | | |
|---|---|---|---|---|---|---|---|---|---|
| | | <1% | 1-5% | 5-40% | Other | match | nonmatch | total | match rate |
| FVIII Activity Level (measured) | <1% | 12 | | | 1 | 12 | 1 | 13 | 92.3 |
| | 1-5% | | 4 | 1 | 3 | 4 | 4 | 8 | 50.0 |
| | 5-40% | | 1 | 1 | 1 | 1 | 2 | 3 | 33.3 |
| | Other | | | 3 | 7 | 7 | 3 | 10 | 70.0 |
| | total | 12 | 5 | 5 | 12 | 24 | 10 | 34 | 70.6 |

FVIII Deficiency match rate = 76.5% (26 specimens out of 34 specimens)

TABLE 20-B3

Analysis Results with Parameter Set B-3

| | | FVIII Activity Level (determined) | | | | FVIII Activity Level Match Rate (%) | | | |
|---|---|---|---|---|---|---|---|---|---|
| | | <1% | 1-5% | 5-40% | Other | match | nonmatch | total | match rate |
| FVIII Activity Level (measured) | <1% | 11 | | | 2 | 11 | 2 | 13 | 84.6 |
| | 1-5% | | 6 | 2 | | 6 | 2 | 8 | 75.0 |
| | 5-40% | | | 2 | 1 | 2 | 1 | 3 | 66.7 |
| | Other | | | 3 | 7 | 7 | 3 | 10 | 70.0 |
| | total | 11 | 6 | 7 | 10 | 26 | 8 | 34 | 76.5 |

FVIII Deficiency match rate = 82.4% (28 specimens out of 34 specimens)

TABLE 20-B4

Analysis Results with Parameter Set B-4

| | | FVIII Activity Level (determined) | | | | FVIII Activity Level Match Rate (%) | | | |
|---|---|---|---|---|---|---|---|---|---|
| | | <1% | 1-5% | 5-40% | Other | match | nonmatch | total | match rate |
| FVIII Activity Level (measured) | <1% | 12 | 1 | | | 12 | 1 | 13 | 92.3 |
| | 1-5% | | 5 | 1 | 2 | 6 | 2 | 8 | 62.5 |
| | 5-40% | | 1 | 2 | | 2 | 1 | 3 | 66.7 |
| | Other | | | | 10 | 10 | 0 | 10 | 100.0 |
| | total | 12 | 7 | 3 | 12 | 30 | 4 | 34 | 88.2 |

FVIII Deficiency match rate = 94.1% (32 specimens out of 34 specimens)

TABLE 21

Analysis Results with Comparative Parameter Set 1

| | | FVIII Activity Level (determined) | | | | FVIII Activity Level Match Rate (%) | | | |
|---|---|---|---|---|---|---|---|---|---|
| | | <1% | 1-5% | 5-40% | Other | match | nonmatch | total | match rate |
| FVIII Activity Level (measured) | <1% | 9 | | | 4 | 9 | 4 | 13 | 69.2 |
| | 1-5% | | 1 | 2 | 5 | 1 | 7 | 8 | 12.5 |
| | 5-40% | | | 1 | 2 | 1 | 2 | 3 | 33.3 |
| | Other | | 1 | 1 | 8 | 8 | 2 | 10 | 80.0 |
| | total | 9 | 2 | 4 | 19 | 19 | 15 | 34 | 55.9 |

FVIII Deficiency match rate = 61.8% (21 specimens out of 34 specimens)

### 6) Difference Caused in Determination Result by Difference in Correlation Evaluation Criteria

In order to check a difference caused in the determination result by a difference in correlation evaluation criteria, comparison was conducted under the following two conditions with differing the correlation evaluation criteria alone. The parameter set A-4 was used.

Correlation Evaluation Criteria 1: Linear regression equations were obtained between all the template specimens and the test specimen with respect to the parameter set, and template specimens having the slope of the regression line in a range from 0.87 to 1.13 were selected therefrom, and a template specimen having the largest correlation coefficient was selected from the selected specimens (the same evaluation criteria as those described above in the section 5)).

Correlation Evaluation Criteria 2: Linear regression equations were obtained between all the template specimens and the test specimen with respect to the parameter set, and a template specimen having the largest correlation coefficient was selected therefrom.

The determination results are shown in Table 22 (Table 22-1 is the same as Table 19-A4). The types of the parameters used in the analysis, and the FVIII deficiency match rate and the FVIII activity level match rate are shown together in Table 23.[0200]

TABLE 22-1

Analysis Results based on Correlation Evaluation Criteria 1

| | | FVIII Activity Level (determined) | | | | FVIII Activity Level Match Rate (%) | | | |
|---|---|---|---|---|---|---|---|---|---|
| | | <1% | 1-5% | 5-40% | Other | match | nonmatch | total | match rate |
| FVIII Activity Level (measured) | <1% | 12 | 1 | | | 12 | 1 | 13 | 92.3 |
| | 1-5% | | 4 | 4 | | 4 | 4 | 8 | 50.0 |
| | 5-40% | | 1 | 2 | | 2 | 1 | 3 | 66.7 |
| | Other | | | | 10 | 10 | 0 | 10 | 100.0 |
| | total | 12 | 6 | 6 | 10 | 28 | 6 | 34 | 82.4 |

FVIII Deficiency match rate = 100% (34 specimens out of 34 specimens)

TABLE 22-2

Analysis Results based on Correlation Evaluation Criteria 2

| | | FVIII Activity Level (determined) | | | | FVIII Activity Level Match Rate (%) | | | |
|---|---|---|---|---|---|---|---|---|---|
| | | <1% | 1-5% | 5-40% | Other | match | nonmatch | total | match rate |
| FVIII Activity Level (measured) | <1% | 11 | 2 | | | 11 | 2 | 13 | 84.6 |
| | 1-5% | | 4 | 4 | | 4 | 4 | 8 | 50.0 |
| | 5-40% | | | 3 | | 3 | 0 | 3 | 100.0 |
| | Other | 1 | | | 9 | 9 | 1 | 10 | 90.0 |
| | total | 12 | 6 | 7 | 9 | 27 | 7 | 34 | 79.4 |

FVIII Deficiency match rate = 97.1% (33 specimens out of 34 specimens)

TABLE 23

| Evaluation Criteria | Parameter Set | Number of Parameters | FVIII Deficiency Match Rate | FVIII Activity Level Match Rate |
|---|---|---|---|---|
| Correlation Evaluation Criteria 1 Slope + Correlation Coefficient | vHg, vTg, vB, vABg, vTBg + Vmax, VmaxT, Amax, AmaxT | 54 | 100 | 82.4 |
| Correlation Evaluation Criteria 2 Correlation Coefficient | vHg, vT,g vB, vABg, vTBg + Vmax, VmaxT, Amax, AmaxT | 54 | 97.1 | 79.4 |

### 7) Determination of FIX Activity of Test Specimen

Among the test specimens, 8 specimens, which had been determined as the “Other” (FVIII > 40%) but were FIX deficient, were determined for FIX activity. The same template specimens shown in Table 24 were used. The parameter set A-1 obtained as described above in the section 4) was used as the parameter set, and the correlation evaluation criteria 1 described in the section 6) was used for the evaluation of correlation. Through similar procedures to those described in the section 5), an FIX activity level match rate and an FIX deficiency match rate were calculated. Evaluation results are shown in Table 25. Thus, the FIX activity level of the test specimens could be determined with a high match rate.

TABLE 24

Constitution of Template Specimens

| Template Specimen | Number of Specimens | Coagulation Factor Activity | | | |
|---|---|---|---|---|---|
| | | <1% | 1-5% | 5-40% | Other |
| Specimens having different FIX activity | 16 | 5 | 4 | 6 | 1 |
| Specimens having other factors | 61 | 0 | 0 | 0 | 61 |
| Total | 77 | 5 | 4 | 6 | 62 |

Specimens having other factors: plasma with deficiency of coagulation factor except FVIII, heparinized plasma, and LA positive plasma

TABLE 25

Analysis Result with Parameter Set A-1

| | | FIX Activity Level (determined) | | | | FIX Activity Level Match Rate (%) | | | |
|---|---|---|---|---|---|---|---|---|---|
| | | <1% | 1-5% | 5-40% | Other | match | nonmatch | total | match rate |
| FIX Activity Level (measured) | <1% | 2 | | | | 2 | 0 | 2 | 100.0 |
| | 1-5% | | 2 | 1 | 1 | 2 | 2 | 4 | 50.0 |
| | 5-40% | | | 2 | | 2 | 0 | 2 | 100.0 |
| | Other | | | | | 0 | 0 | 0 | |
| | total | 2 | 2 | 3 | 1 | 6 | 2 | 8 | 75.0 |

FIX Deficiency match rate = 87.5% (7 specimens out of 8 specimens)

The embodiments of the present invention have been exemplified so far, and it is noted that these embodiments are merely examples and do not intend to limit the scope of the invention. The above-described embodiments can be practiced in other various forms, and can be variously omitted, replaced and modified without departing from the spirit of the present invention. Besides, the embodiments can be practiced with the constitutions, the numerical values and the like appropriately modified. In addition, a combination of some of these examples can result in a new embodiment.

1. A blood analysis method, comprising:
(1) acquiring coagulation reaction data on a subject blood specimen;
(2) calculating a parameter related to a centroid point from a differential curve of the coagulation reaction data; and
(3) evaluating coagulation properties of the blood specimen using the parameter related to the centroid point.

2. The method according to claim 1, wherein the centroid point is at least one selected from the group consisting of a centroid point in a prescribed region of a primary differential curve of a coagulation reaction curve of the blood specimen, and a centroid point in a prescribed region of a secondary differential curve of the coagulation reaction curve.

3. The method according to claim 2,
wherein the centroid point in the prescribed region of the primary differential curve is represented by coordinates (vTg, vHg) defined by a centroid time vTg and a centroid height vHg, and
the parameter related to the centroid point includes one or more parameters of the centroid point related to the centroid point in the prescribed region of the primary differential curve selected from the group consisting of the centroid height vHg, a centroid peak width vWg, a B flattening vABg, a W flattening vAWg, and a W time rate vTWg, wherein,
assuming that the primary differential curve is F(t) (wherein t is time), that times when F(t) has a prescribed value X are t1 and t2 (wherein t1 < t2), and that when n = t2 - t1 + 1, and b = X, vTg and vHg are represented by the following expressions:
$$\text{v} T \text{g} = \frac{{\sum }_{i = t 1}^{t 2} \left(i \times F \left(i\right)\right)}{{\sum }_{i = t 1}^{t 2} F \left(i\right)} \qquad \text{(1)}$$
$$\text{v} H \text{g} = \frac{\left({\sum }_{i = t 1}^{t 2} F \left(i\right) ∗ F \left(i\right)\right) - \left(n ∗ b ∗ b\right)}{2 ∗ \left\{\left({\sum }_{i = t 1}^{t 2} F \left(i\right)\right) - n ∗ b\right\}} \qquad \text{(2)}$$
wherein vWg represents a time length satisfying F(t) ≥ vHg in time from t1 to t2,
vABg represents a ratio between vHg and vB, wherein vB represents a time length satisfying F(t) ≥ X in time from t1 to t2,
vAWg represents a ratio between vHg and vWg, and
vTWg represents a ratio between vTg and vWg.

4. The method according to claim 3, wherein the prescribed value X is a value corresponding to from 0.5% to 99% of a maximum value of the primary differential curve F(t).

5. The method according to claim 2, wherein the centroid point in the prescribed region of the secondary differential curve includes one or more selected from the group consisting of a centroid point in a prescribed region of a positive peak of the secondary differential curve, and a centroid point in a prescribed region of a negative peak of the secondary differential curve.

6. The method according to claim 5,
wherein the centroid point in the prescribed region of the positive peak of the secondary differential curve is represented by coordinates (pTg, pHg) defined by a centroid time pTg and a centroid height pHg, and
the parameter related to the centroid point includes one or more parameters related to the centroid point in the prescribed region of the positive peak of the secondary differential curve selected from the group consisting of the centroid height pHg, a centroid peak width pWg, a B flattening pABg, a W flattening pAWg, and a W time rate pTWg, wherein,
assuming that the secondary differential curve is F′(t) (wherein t is time), that times when F′(t) has a prescribed value X′ are t1 and t2 (wherein t1 < t2), and that when n = t2 - t1 + 1, and b′ = X′, pTg and pHg are represented by the following expressions:
$$\text{p} T \text{g} = \frac{{\sum }_{i = t 1}^{t 2} \left(i \times {F}^{′} \left(i\right)\right)}{{\sum }_{i = t 1}^{t 2} {F}^{′} \left(i\right)} \qquad \text{(1)}$$
$$\text{p} H \text{g} = \frac{\left({\sum }_{i = t 1}^{t 2} {F}^{′} \left(i\right) ∗ {F}^{′} \left(i\right)\right) - \left(n ∗ {b}^{′} ∗ {b}^{′}\right)}{2 ∗ \left\{\left({\sum }_{i = t 1}^{t 2} {F}^{′} \left(i\right)\right) - n ∗ {b}^{′}\right\}} \qquad \text{(2)}$$
wherein pWg represents a time length satisfying F′(t) ≥ pHg in time from t1 to t2,
pABg represents a ratio between pHg and pB, wherein pB represents a time length satisfying F′(t) ≥ X′ in time from t1 to t2,
pAWg represents a ratio between pHg and pWg, and
pTWg represents a ratio between pTg and pWg.

7. The method according to claim 5,
wherein the centroid point in the prescribed region of the negative peak of the secondary differential curve is represented by coordinates (mTg, mHg) defined by a centroid time mTg and a centroid height mHg, and
the parameter related to the centroid point includes one or more parameters related to the centroid point in the prescribed region of the negative peak of the secondary differential curve selected from the group consisting of the centroid height mHg, a centroid peak width mWg, a B flattening mABg, a W flattening mAWg, and a W time rate mTWg,
wherein,
assuming that the secondary differential curve is F′(t) (wherein t is time), that times when F′(t) has a prescribed value X″ are t1 and t2 (wherein t1 < t2), and that when n = t2 - t1 + 1, and b″ = X″, mTg and mHg are represented by the following expressions:
$$\text{m} T \text{g} = \frac{{\sum }_{i = t 1}^{t 2} \left(i \times {F}^{′} \left(i\right)\right)}{{\sum }_{i = t 1}^{t 2} {F}^{′} \left(i\right)} \qquad \text{(1)}$$
$$\text{m} H \text{g} = \frac{\left({\sum }_{i = t 1}^{t 2} {F}^{′} \left(i\right) ∗ {F}^{′} \left(i\right)\right) - \left(n ∗ {b}^{″} ∗ {b}^{″}\right)}{2 ∗ \left\{\left({\sum }_{i = t 1}^{t 2} {F}^{′} \left(i\right)\right) - n ∗ {b}^{″}\right\}} \qquad \text{(2)}$$
wherein mWg represents a time length satisfying F′(t) ≤ mHg in time from t1 to t2,
mABg represents a ratio between mHg and mB, wherein mB represents a time length satisfying F′(t) ≤ X″ in time from t1 to t2,
mAWg represents a ratio between mHg and mWg, and
mTWg represents a ratio between mTg and mWg.

8. The method according to claim 6, wherein the prescribed value X′ is a value corresponding to from 0.5% to 99% of a maximum value of the secondary differential curve F′(t).

9. The method according to claim 7, wherein the prescribed value X″ is a value corresponding to from 0.5% to 99% of a minimum value of the secondary differential curve F′(t).

10. The method according to claim 1, wherein the evaluation of the coagulation properties is measurement of a concentration of a coagulation factor.

11. The method according to claim 10, wherein the coagulation factor is at least one selected from the group consisting of coagulation factor VIII and coagulation factor IX.

12. The method according to claim 1, wherein the evaluation of the coagulation properties is evaluation of presence or degree of coagulation abnormality.

13. The method according to claim 12, wherein the coagulation abnormality is hemophilia A or hemophilia B.

14. The method according to claim 1, wherein the evaluation of the coagulation properties is evaluation of a coagulation time elongation factor.

15. The method according to claim 14, wherein the evaluation of the elongation factor is evaluation of which of coagulation factor deficiency, a lupus anticoagulant, and a coagulation factor inhibitor is the elongation factor.

16. The method according to claim 1, wherein the evaluation of the coagulation properties is measurement of a titer of a coagulation factor inhibitor.

17. The method according to claim 16, wherein the coagulation factor inhibitor is a coagulation factor VIII inhibitor.

18. The method according to claim 14,
wherein the (1) comprises:
preparing a mixed specimen by mixing a subject blood specimen and a normal blood specimen;
heating the mixed specimen, and acquiring coagulation reaction data of the heated mixed specimen; and
acquiring coagulation reaction data of the mixed specimen unheated,
the (2) comprises:
calculating, as a first parameter, a parameter related to the centroid point of the mixed specimen unheated; and
calculating, as a second parameter, a parameter related to the centroid point of the heated mixed specimen, and
the (3) comprises:
evaluating coagulation properties of the subject blood specimen based on a ratio or a difference between the first parameter and the second parameter.

19. The method according to claim 18, wherein the heating is performed at 30° C. or more and 40° C. or less for 2 to 30 minutes.

20. The method according to claim 10,
wherein the (2) comprises:
acquiring a parameter set including a parameter group consisting of parameters related to a centroid point each of which are calculated from different regions of the differential curve,
the (3) comprises:
comparing the parameter set of the subject blood specimen with a corresponding parameter set of a template blood specimen, and
evaluating, based on a result of the comparing, presence or degree of coagulation abnormality in the subject blood specimen, and
the template blood specimen is a blood specimen in which presence or degree of the coagulation abnormality is known.

21. The method according to claim 20, wherein the number of the different regions is from 5 to 50.

22. A program for performing the blood analysis method according to claim 1.

23. An apparatus for performing the blood analysis method according to claim 1.
